(19) United States
(12) Patent Application Publication
(10) Pub. No.: US 2026/0022353 A1
(43) Pub. Date: Jan. 22, 2026

(54) TRANSPOSASE POLYNUCLEOTIDES AND USES THEREOF

(71) Applicant: Poseida Therapeutics, Inc., San Diego, CA (US)

(72) Inventors: Blair B. MADISON, San Diego, CA (US); Johannes SCHWERK, San Diego, CA (US)

(21) Appl. No.: 19/349,741

(22) Filed: Oct. 3, 2025

Related U.S. Application Data
(63) Continuation of application No. PCT/US2024/022985, filed on Apr. 4, 2024.
(60) Provisional application No. 63/494,303, filed on Apr. 5, 2023.
(60) Provisional application No. 63/610,715, filed on Dec. 15, 2023.

(51) Int. Cl. C12N 9/12; C12N 15/88

(52) U.S. Cl. CPC C12N 9/1241; C12N 15/88

(57) ABSTRACT

Provided are polynucleotides for expressing transposases, in particular, polynucleotide mRNAs for expressing piggyBac transposases comprising a piggyBac transposase coding sequence comprising hyperactive mutations and modified 5′- and 3′-UTR sequences to enhance piggyBac transposase expression, integration/excision activity and/or reduce in vivo immunogenicity of the encoded transposase. Also provided are methods for the delivery, including in vivo delivery, of an exogenous nucleic acid to a cell.

### CROSS-REFERENCE TO RELATED APPLICATIONS

The present application is a continuation of International Patent Application No. PCT/US2024/022985, filed Apr. 4, 2024, which claims the benefit of U.S. Provisional Patent Applications No. 63/494,303 filed Apr. 5, 2023 and No. 63/610,715 filed Dec. 15, 2023, each of which is incorporated herein by reference in its entirety.

### REFERENCE TO SEQUENCE LISTING SUBMITTED ELECTRONICALLY

The instant application contains a Sequence Listing, which has been submitted electronically in XML file format, and is herein incorporated by reference into the specification in its entirety. The XML file containing the Sequence Listing XML is named “000218-0142-101-SL.xml,” was created on Oct. 3, 2025 and is 94,561 bytes in size.

### FIELD

This disclosure generally relates to polynucleotides for expressing transposases, in particular, polynucleotide mRNAs for expressing piggyBac transposases comprising a piggyBac transposase coding sequence comprising hyperactive mutations and modified 5′-and 3-'UTR sequences to enhance piggyBac transposase expression, integration/excision activity and/or reduce in vivo immunogenicity of the encoded transposase.

### BACKGROUND

Transposases may be used to introduce non-endogenous DNA sequences into genomic DNA, and are in many ways advantageous to other methods gene editing. However, there remains an unmet need for transposases possessing enhanced integration/excision activity and reduced in vivo immunogenicity for in vivo delivery of nucleic acids. It has previously been reported that incorporation of human cytochrome b-245 alpha polypeptide (CYBA) UTRs into a mRNA sequence significantly increased protein levels for certain mRNAs without altering mRNA stability (see e.g., Ferizi, M., Aneja, M., Balmayor, E. et al. Sci Rep 6, 39149 (2016)). Importantly, many miRNAs have been shown to be expressed in hematopoietic lineages and to act as pivotal regulators of transcriptional programs for normal hematopoiesis, including HSC self-renewal, differentiation and functioning (e.g., see Lu et al. (2013) Cell Res 23, 1356-1368 (2013).

### SUMMARY

In one aspect, provided herein is a polynucleotide, comprising in the 5′ to 3′ direction: (i) a hemoglobin beta (HBB) 5′-UTR, (ii) a sequence encoding a nuclear localization signal (NLS), (iii) a nucleic acid sequence encoding a piggyBac transposase, (iv) a 3′-UTR comprising one or more nucleic acid sequences comprising a human cytochrome b-245 alpha polypeptide (CYBA) 3′-UTR element and one or more miR-142-3p binding sites, and (v) a polyA tail. In some embodiments, the piggyBac transposase comprises the five hyperactive mutations selected 130V; G165S; M226F, M282V and N538K (i.e., the amino acid sequence set forth in SEQ ID NO: 14). In some embodiments, the nucleic acid sequence encoding the piggyBac transposase comprises the nucleic acid sequence set forth in SEQ ID NO: 2.

In some embodiments, the NLS is an SV40 NLS comprising the amino acid sequence set forth in SEQ ID NO: 8.

In some embodiments, the one or more CYBA 3′-UTR element(s) each comprises the nucleic acid sequence set forth in SEQ ID NO: 3. In some embodiments, the 3′-UTR comprises at least two tandem nucleic acid sequences encoding a CYBA 3′-UTR element which are separated by a linker sequence. In some embodiments, the tandem nucleic acid sequences encoding a CYBA 3′-UTR element each comprise the nucleic acid sequence set forth in SEQ ID NO. 4.

In some embodiments, the one or more miR-142-3p binding sites each comprise the nucleic acid sequence set forth in SEQ ID NO: 5. In some embodiments, each of the one or more miR-142-3p binding sites comprises the nucleic acid sequence ACACTAC. In some embodiments, the 3′-UTR comprises four miR-142-3p binding sites. In some embodiments, the polynucleotide further comprises a linker sequence located between each of the four miR-142-3p binding sites. In some embodiments, the linker sequence comprises the nucleic acid sequence set forth in SEQ ID NO. 6.

In some embodiments, the HBB 5′-UTR comprises the nucleic acid sequence set forth in SEQ ID NO: 1.

In some embodiments, the poly A tail is an 80X polyA tail comprising the nucleic acid sequence set forth in SEQ ID NO: 7.

In some embodiments, the polynucleotide comprises the nucleic acid sequence set forth in SEQ ID NO: 10.

In some embodiments, the polynucleotide is a DNA molecule. In some embodiments, the polynucleotide is an RNA molecule. In some embodiments, the RNA molecule is an mRNA molecule. In some embodiments, the mRNA comprises a 5′-CAP. In some embodiments, the 5′CAP is a 5′ CleanCap.

In another aspect, provided herein is a lipid nanoparticle (LNP) composition comprising a polynucleotide described herein.

In another aspect, provided herein is a method for the delivery of an exogenous nucleic acid to a cell, comprising: introducing into the cell a DNA transposon comprising an exogenous nucleic acid; and an mRNA comprising in 5′ to 3′ direction: (i) a HBB 5′-UTR, a nucleic acid sequence encoding an NLS, (ii) a nucleic acid sequence encoding a piggyBac transposase comprising five hyperactive mutations, (iii) a 3′-UTR comprising two or more tandem nucleic acid sequences comprising a CYBA 3′-UTR element and four miR-142-3p binding sites, and (iv) a polyA tail; and wherein the piggyBac transposase is expressed in the cell and integrates the transposon comprising the exogenous nucleic acid at a TTAA integration site in the cell genome, wherein the expressed piggyBac transposase exhibits enhanced integration and/or excision activity compared to a piggyBac transposase comprising four or fewer hyperactive mutations.

In another aspect, provided herein is a method for the in vivo delivery of an exogenous nucleic acid to a cell in a subject, comprising: co-introducing into the subject a DNA transposon comprising an exogenous nucleic acid; and an mRNA comprising in the 5′ to 3′ direction: a HBB 5′-UTR, a NLS coding sequence, a piggyBac transposase coding sequence comprising five hyperactive mutations, a 3′-UTR comprising tandem CYBA 3′-UTR element upstream of the sequences for four miR-142-3p binding sites, and a polyA tail; and wherein a cell in the subject uptakes the transposon and expresses the piggyBac transposase in the cell and integrates the transposon comprising the exogenous nucleic acid at a TTAA integration site in the cell genome of the subject, wherein the expressed piggyBac transposase exhibits reduced immunogenicity in the subject compared to a mRNA encoding a piggyBac transposase lacking the sequences for four miR-142-3p binding sites.

### BRIEF DESCRIPTION OF DRAWINGS

FIG. 1A shows a schematic illustrating an illustrative polynucleotide for expressing a piggyBac transposase comprising, in the 5′ to 3′ direction: a HBB 5′-UTR; a coding sequence for a nuclear localization sequence (NLS); a piggyBac transposase coding sequence encoding four hyperactive mutations; a HBB 3′UTR, and a polyA tail. FIG. 1B shows a schematic illustrating a polynucleotide for expressing a piggyBac transposase comprising, in the 5′ to 3′ direction: a HBB 5′-UTR; a coding sequence for a nuclear localization sequence (NLS); a piggyBac transposase coding sequence encoding five hyperactive mutations; a 3′UTR comprising for tandem CYBA 3′-UTR sequences and sequences for four miR-142-3p binding sites, and a polyA tail.

FIG. 2 is an illustration of the GFP episomal excision reporter construct.

FIG. 3A shows a schematic of two transposase expressing polynucleotides comprising, in the 5′ to 3′ direction: a HBB 5′-UTR; a NLS coding sequence; a piggyBac transposase coding sequence encoding four hyperactive mutations; a HBB 3′-UTR, and a poly A tail, and a polynucleotide comprising, in the 5′ to 3′ direction: a HBB 5′-UTR; a coding sequence for a nuclear localization sequence (NLS); a piggyBac transposase coding sequence encoding four hyperactive mutations; a tandem CYBA 3′-UTR. SPBv3.1-HBB is the same as shown in FIG. 1. SPB-HBB/2xCYBA is an intermediate construct which lacks the fifth hypermutation (M226F) and the miR-142-3p binding sites compared to the construct shown in FIG. 1.

FIG. 3B shows results of an excision reporter assay showing a time course of GFP expression in HepG2 cells transposed using transposase expressing polynucleotides of FIG. 3A, or a catalytically dead transposase (“CD-SPB”) as a negative control.

FIG. 4 shows a graph of Day 6 serum Factor VIII (hFVIII) levels (as % normal level) of juvenile BALB/c mice co-administered an LNP compositions encapsulating a DNA transposon comprising a FVIII expression cassette and an LNP composition encapsulating one of the mRNA transposase expressing polynucleotides of FIG. 3A, or a catalytically dead (CD) transposase as a negative control.

FIG. 5 shows a graph of Day 6 serum Factor VIII (hFVIII) levels (as % normal level) of adult C57BL/6 mice co-administered an LNP compositions encapsulating a DNA transposon comprising a FVIII expression cassette and an LNP composition encapsulating one of the mRNA transposase expressing polynucleotides of FIG. 3A.

FIG. 6 shows results of an excision reporter assay showing a 42 hr time course of GFP expression in HepG2 cells transposed using transposase expressing polynucleotides lacking (“w/o”) or comprising (“+miR-142-3p BS”) four miR-142-3p binding sites in the 3′-UTR or a catalytically dead transposase (“CD-SPB”) as a negative control.

FIG. 7 shows results of a GFP reporter expression assay showing an 18 hr time course of GFP expression in K562 cells (a hematopoietic cell) nucleofected with GFP expressing polynucleotides lacking (“w/o”) or comprising (“+miR-142-3p BS”) four miR-142-3p binding sites in the 3′-UTR.

FIG. 8 shows results of an excision reporter assay showing a 42 hr time course of GFP expression in K562 cells transposed using transposase expressing polynucleotides lacking (“w/o”) or comprising (“+miR-142-3p BS”) four miR-142-3p binding sites in the 3′-UTR.

FIG. 9 shows a graph of Day 6 serum Factor VIII (hFVIII) levels (as % normal level) of juvenile BALB/c mice co-administered an LNP compositions encapsulating a DNA transposon comprising a FVIII expression cassette and an LNP composition encapsulating one of the mRNA transposase expressing polynucleotides of FIG. 3A or the mRNA transposase expressing polynucleotides of FIG. 3A further comprising sequences for four miR-142-3p binding sites in the 3′-UTR, or a catalytically dead transposase as a negative control.

FIG. 10 shows the number of piggyBac transposase reactive T-cells (as # of IFN gamma positive cells/10e6 splenocytes) at Day 28 in juvenile BALB/c mice co-administered an LNP composition encapsulating a DNA transposon comprising a FVIII expression cassette and an LNP composition encapsulating one of the mRNA transposase expressing polynucleotides of FIG. 3A, the mRNA transposase expressing polynucleotides of FIG. 3A further comprising sequences for four miR-142-3p binding sites in the 3′-UTR, or a catalytically dead transposase as a negative control.

FIG. 11 shows the number of piggyBac transposase reactive T-cells (as #of IFN gamma positive cells/10e6 splenocytes) at Day 21 in adult C57BL/6 mice co-administered an LNP compositions encapsulating a DNA transposon comprising a FVIII expression cassette and an LNP composition encapsulating an one of the mRNA transposase expressing polynucleotides of FIG. 3A, the mRNA transposase expressing polynucleotides of FIG. 3A further comprising sequences for four miR-142-3p binding sites in the 3′UTR, or a catalytically dead transposase as a negative control. ‘1 dose’ groups received LNP compositions on Day 1 of the study; ‘3 dose’ groups received LNP compositions repeatedly on Days 1, 7, and 14 of the study.

FIG. 12 shows a schematic depiction of the dual reporter plasmid design used to confirm the rates of excision and integration using each mutant transposon. Using an H-2Kk GFP transposon reporter (Reporter 1), an increase in H-2Kk expression is observed if there is an increase in excision of the transposon. Using Reporter 2, an increase in GFP expression is observed if there is an increase in the integration of the transposon. In an alternative design of Reporter 2, an increase in Firefly luciferase expression is observed if there is an increase in excision of the transposon and an increase in NanoLuc is observed if there is an increase in the integration of the transposon.

FIG. 13 shows a schematic depiction of an H-2Kk GFP transposon reporter (Reporter 1). Structural features of the transposon are shown both in a circular map and a linear map. An increase in H-2Kk expression is observed if there is an increase in excision of the transposon and an increase in GFP is observed if there is an increase in integration of the transposon.

FIG. 14 is a schematic depiction of a Firefly luciferase Nano Luc transposon reporter. Structural features of the transposon are shown both in a circular map and a linear map. Firefly luciferase expression is observed if there is an increase in excision of the transposon and an increase in NanoLuc is observed if there is an increase in the integration of the transposon.

FIGS. 15A and 15B show results of a luciferase dual integration/excision reporter assay in K562 cells and 293T cells, respectively, using a DNA transposon comprising luciferase dual integration/excision reporter construct comprising wild type LE and RE ITRs, or a 35TCC LE and a wild type RE ITR, and DNA plasmid comprising transposase expressing polynucleotides encoding a piggyBac transposase comprising 4 (“SPB”) or 5 (“Hyper”) hyperactive mutations, or no transposase expressing polynucleotides encoding a piggyBac transposase (no SPB) as a negative control.

FIG. 16 shows results of a luciferase dual integration/excision reporter assay in juvenile BALB/c mice co-administered an LNP compositions encapsulating a DNA transposon comprising luciferase dual integration/excision reporter construct comprising wild type LE and RE ITRs or a 35TCC LE and a wild type RE ITR, and an LNP composition encapsulating an mRNA expressed from one of the mRNA transposase expressing polynucleotides of FIG. 1, or a catalytically dead transposase as a negative control.

FIG. 17A-17E show a comparison of the activity of two version of SPB as measured by transposition efficacy, B2M knockout and T cell expansion.

### DETAILED DESCRIPTION

Provided herein are polynucleotides for expressing transposases, in particular, polynucleotide mRNAs for expressing piggyBac transposases comprising a piggyBac transposase coding sequence comprising hyperactive mutations and modified 5′ and 3′UTR sequences to enhance piggyBac transposase expression, activity and reduce in vivo immunogenicity of the encoded transposase.

### PiggyBac Transposase Expressing Polynucleotides

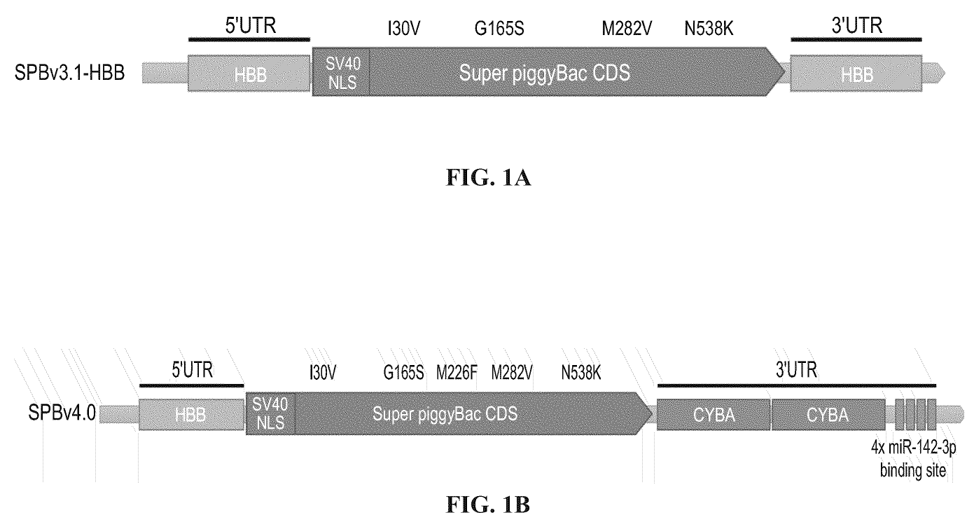

In certain aspects of the disclosure, the polynucleotide encoding the transposase coding sequence is a polynucleotide comprising in the 5′ to 3′ direction: (i) a HBB 5′-UTR, (ii) a nucleic acid sequence encoding a SV40 NLS, (iii) a nucleic acid sequence encoding a piggyBac transposase comprising five hyperactive mutations, (iv) a 3′-UTR comprising a tandem CYBA 3′-UTR element upstream of a nucleic acid sequences encoding four miR-142-3p binding sites, and (v) an 80X polyA tail (FIG. 1).

In certain aspects, the 5′-UTR is a HBB 5′-UTR. In certain aspects, the HBB 5′-UTR sequence comprises, or consists essentially of the nucleic acid sequence: ACATTTGCTTCTGACACAACTGTGTTCACTAGCAACCTCAAACAGACACC (SEQ ID

NO. 1). In some embodiment, the HBB 5′UTR sequence comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 1.

In certain aspects, the nucleic acid sequence encodes a piggyBac transposase comprises the following five hyperactive mutations: 130V; G165S; M226F, M282V and N538K (e.g., the piggyBac transposase comprising the sequence set forth in SEQ ID NO: 45).

In some embodiments, a piggyBac transposase comprising the five hyperactive mutations I30V; G165S; M226F, M282V and N538K comprises the sequence of SEQ ID NO: 45(the underlined bold sequence is a nuclear localization sequence and may be omitted; numbering of the residues for mutation purposes begin at residue 12).

(SEQ ID NO: 45)
MAPKKKRKVGGGGSSLDDEHILSALLQSDDELVGEDSDSEVSDHVSEDDV
QSDTEEAFIDEVHEVQPTSSGSEILDEQNVIEQPGSSLASNRILTLPQRT
IRGKNKHCWSTSKSTRRSRVSALNIVRSQRGPTRMCRNIYDPLLCFKLFF
TDEIISEIVKWTNAEISLKRRESMTSATFRDTNEDEIYAFFGILVMTAVR
KDNHMSTDDLFDRSLSMVYVSVMSRDRFDFLIRCLRFDDKSIRPTLREND
VFTPVRKIWDLFIHQCIQNYTPGAHLTIDEQLLGFRGRCPFRVYIPNKPS
KYGIKILMMCDSGTKYMINGMPYLGRGTQTNGVPLGEYYVKELSKPVHGS
CRNITCDNWFTSIPLAKNLLQEPYKLTIVGTVRSNKREIPEVLKNSRSRP
VGTSMFCFDGPLTLVSYKPKPAKMVYLLSSCDEDASINESTGKPQMVMYY
NQTKGGVDTLDQMCSVMTCSRKTNRWPMALLYGMINIACINSFIIYSHNV
SSKGEKVQSRKKFMRNLYMSLTSSFMRKRLEAPTLKRYLRDNISNILPKE
VPGTSDDSTEEPVMKKRTYCTYCPSKIRRKANASCKKCKKVICREHNIDM
CQSCF.

In some embodiment, the piggyBac transposase comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 45

In certain aspects, the nucleic acid sequence encoding the piggyBac transposase comprising the mutations 130V; G165S; M226F, M282V and N538K comprises, or consists of the sequence:

(SEQ ID NO. 2)
ATGGCTCCCAAGAAGAAGCGGAAAGTTGGCGGCGGAGGCAGCAGCCTGGA
TGATGAGCATATTCTGAGCGCCCTGCTGCAGAGCGACGATGAACTCGTGG
GCGAAGATAGCGACAGCGAGGTGTCCGATCACGTGTCCGAGGATGACGTG
CAGTCCGATACCGAGGAAGCCTTCATCGACGAGGTGCACGAAGTGCAGCC
TACAAGCAGCGGCAGCGAGATCCTGGACGAGCAGAATGTGATCGAGCAGC
CAGGATCTAGCCTGGCCAGCAACAGAATCCTGACACTGCCCCAGAGAACC
ATCCGGGGCAAGAACAAGCACTGCTGGTCCACCAGCAAGAGCACCAGACG
GTCTAGAGTGTCTGCCCTGAACATCGTGCGAAGCCAGAGGGGCCCTACCA
GAATGTGCCGGAACATCTACGACCCTCTGCTGTGCTTCAAGCTGTTCTTC
ACCGACGAGATCATCTCCGAGATCGTGAAGTGGACCAACGCCGAGATCAG
CCTGAAGCGGAGAGAATCCATGACCAGCGCCACCTTCAGAGACACCAACG
AGGACGAGATCTACGCCTTCTTCGGCATCCTGGTCATGACAGCCGTGCGG
AAGGACAACCACATGAGCACCGACGACCTGTTCGACCGCAGCCTGTCTAT
GGTGTACGTGTCCGTGATGAGCCGGGACAGATTCGACTTCCTGATCCGGT
GCCTGCGGTTCGACGACAAGTCCATCAGACCCACACTGCGCGAGAACGAC
GTGTTCACACCTGTGCGGAAGATCTGGGACCTGTTCATCCACCAGTGCAT
CCAGAACTACACCCCTGGCGCTCACCTGACCATCGACGAACAGCTGCTGG
GCTTCAGAGGCAGATGCCCCTTCAGAGTGTACATCCCCAACAAGCCCTCT
AAGTACGGCATCAAGATCCTGATGATGTGCGACAGCGGCACCAAGTACAT
GATCAACGGCATGCCCTACCTCGGCAGAGGCACCCAAACAAATGGCGTGC
CACTGGGCGAGTACTACGTGAAAGAACTGAGCAAGCCTGTGCACGGCAGC
TGCAGAAACATCACCTGTGACAACTGGTTTACCAGCATTCCCCTGGCCAA
GAACCTGCTGCAAGAACCCTACAAGCTGACAATCGTGGGCACCGTGCGGA
GCAACAAGAGGGAAATTCCCGAGGTGCTGAAGAACTCTCGGAGCAGACCT
GTGGGCACCAGCATGTTCTGCTTCGACGGACCTCTGACACTGGTGTCCTA
CAAGCCCAAGCCTGCCAAGATGGTGTACCTGCTGAGCAGCTGTGACGAGG
ACGCCAGCATCAATGAGAGCACCGGCAAGCCCCAGATGGTCATGTACTAC
AACCAGACCAAAGGCGGCGTGGACACCCTGGATCAGATGTGCAGCGTGAT
GACCTGCAGCAGAAAGACCAACAGATGGCCCATGGCTCTGCTGTACGGCA
TGATCAATATCGCCTGCATCAACAGCTTCATCATCTACAGCCACAACGTG
TCCAGCAAGGGCGAGAAGGTGCAGAGCCGGAAGAAATTCATGCGGAACCT
GTACATGAGCCTGACCAGCAGCTTCATGAGAAAGCGGCTGGAAGCCCCTA
CACTGAAGAGATACCTGCGGGACAACATCAGCAACATCCTGCCTAAAGAG
GTGCCCGGCACCAGCGACGATAGCACAGAGGAACCCGTGATGAAGAAGAG
GACCTACTGCACCTACTGTCCCAGCAAGATCCGGCGGAAGGCCAACGCCA
GCTGCAAAAAGTGCAAGAAAGTGATCTGCCGCGAGCACAACATCGATATG
TGCCAGAGCTGCTTCtga.

In some embodiment, the nucleic acid sequence encoding the piggyBac transposase comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 2.

In certain aspects, the nucleic acid sequence encodes a piggyBac transposase comprising the following four hyperactive mutations: 130V, G165S, M282V and N538K.

In some embodiments, a piggyBac transposase comprising the four hyperactive mutations I30V, G165S, M282V and N538K comprises the sequence of SEQ ID NO: 46 (the underlined bold sequence is a nuclear localization sequence and may be omitted; numbering of the residues for mutation purposes begins at residue 12).

(SEQ ID NO: 46)
MAPKKKRKVGGGGSSLDDEHILSALLQSDDELVGEDSDSEVSDHVSEDDV
QSDTEEAFIDEVHEVQPTSSGSEILDEQNVIEQPGSSLASNRILTLPQRT
IRGKNKHCWSTSKSTRRSRVSALNIVRSQRGPTRMCRNIYDPLLCFKLFF
TDEIISEIVKWTNAEISLKRRESMTSATFRDTNEDEIYAFFGILVMTAVR
KDNHMSTDDLFDRSLSMVYVSVMSRDRFDFLIRCLRMDDKSIRPTLREND
VFTPVRKIWDLFIHQCIQNYTPGAHLTIDEQLLGFRGRCPFRVYIPNKPS
KYGIKILMMCDSGTKYMINGMPYLGRGTQTNGVPLGEYYVKELSKPVHGS
CRNITCDNWFTSIPLAKNLLQEPYKLTIVGTVRSNKREIPEVLKNSRSRP
VGTSMFCFDGPLTLVSYKPKPAKMVYLLSSCDEDASINESTGKPQMVMYY
NQTKGGVDTLDQMCSVMTCSRKTNRWPMALLYGMINIACINSFIIYSHNV
SSKGEKVQSRKKFMRNLYMSLTSSFMRKRLEAPTLKRYLRDNISNILPKE
VPGTSDDSTEEPVMKKRTYCTYCPSKIRRKANASCKKCKKVICREHNIDM
CQSCF.

In some embodiment, the piggyBac transposase comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 46.

In certain aspects, the NLS sequence comprises, or consists essential of a nucleic acid sequence encoding the amino acid sequence PKKKRKV (SEQ ID NO: 8). In certain aspects, the nucleic acid encoding the NLS comprises, or consists of the nucleic acid sequence: CCCAAGAAGAAGCGGAAAGTT (SEQ ID NO: 9).

In certain aspects, the 80X poly A tail comprises, or consists of the sequence:

(SEQ ID NO: 7)
AAAAAAAAAAAAAAAAAAAAAAAAAAAAAAAAAAAAAAAAAAAAAAAAAA
AAAAAAAAAAAAAAAAAAAAAAAAAAAAAA.

In certain aspects, the polynucleotide for expressing a piggyBac transposase comprises the nucleic acid sequence set forth in SEQ ID NO: 10. In some embodiment, the polynucleotide for expressing a piggyBac transposase comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 10.

### CYBA 3′-UTR Element

In certain aspects, the transposase expressing polynucleotides comprise one or more nucleic acid sequence(s) encoding a CYBA 3′-UTR element.

In certain aspects, the nucleic acid encoding a CYBA 3′-UTR element comprises, or consists of the nucleic acid sequence:

(SEQ ID NO. 3)
CCTCGCCCCGGACCTGCCCTCCCGCCAGGTGCACCCACCTGCAATAAATG
CAGCGAAGCCGGGA.

In some embodiment, the nucleic acid encoding a CYBA 3′-UTR element comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 3.

In certain aspects, the polynucleotide comprises tandem nucleic acid sequences each encoding a CYBA 3′-UTR element. In some embodiments, two tandem nucleic acid sequences encoding CYBA 3′-UTR elements are separated via a linker sequence. In certain aspects, the tandem nucleic acid sequences encoding CYBA 3′-UTR elements separated by a linker sequence together comprises, or consists of the sequence:

(SEQ ID NO. 4)
CCTCGCCCCGGACCTGCCCTCCCGCCAGGTGCACCCACCTGCAATAAATG
CAGCGAAGCCGGGAGAATTCCCTCGCCCCGGACCTGCCCTCCCGCCAGGT
GCACCCACCTGCAATAAATGCAGCGAAGCCGGGA.

miR-142-3P Binding Sites

The transposase encoding polynucleotides described herein comprise one or more miR-142-3p binding sites in the 3′-UTR of the encoded mRNAs. microRNAs (miRNAs) are a group of non-coding RNAs (˜22 nt) that can silence gene expression by binding to the 3′ untranslated region (UTR) or to the coding region of target mRNAs to promote mRNA destabilization or inhibit protein translation.

In some embodiments, the miR-142-3p binding site comprises the sequence ACACTAC. In certain aspects, the miR-142-3p sequence comprises, or consists of the nucleic acid sequence: TCCATAAAGTAGGAAACACTACA (SEQ ID NO. 5).

In certain aspects, the 3′-UTR comprises four miR-142-3p binding sites. In certain aspects, the four miR-142-3p target sequences are separated via three linker sequences. In certain aspects, the four miR-142-3p target sequences separated via the three linker sequences comprises, or consists of the sequence:

(SEQ ID NO. 6)
TCCATAAAGTAGGAAACACTACACGATTCCATAAAGTAGGAAACACTACA
ACCGGTTCCATAAAGTAGGAAACACTACATCACTCCATAAAGTAGGAAAC
ACTACA.

piggyBac ITR sequences

PiggyBac transposases recognize transposon-specific inverted terminal repeat sequences (ITRs) on the ends of the transposon, and inserts the contents between the ITRs at the sequence 5′-TTAA-3′ within a chromosomal site (a TTAA target sequence) or at the sequence 5′-TTAT-3′ within a chromosomal site (a TTAT target sequence). The target sequence of the PB or a piggyBac-like (PBL) transposon can comprise or consist of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. The PB transposon system has no payload limit for the genes of interest (i.e., no limit as to the size of the gene of interest) that can be included between the ITRs.

In some aspects, the DNA transposon comprises a piggyBac ITR sequence. In some aspects, the DNA transposon comprises a first piggyBac ITR sequence, wherein the first piggyBac sequence is a piggyBac left end (LE) ITR sequence. In some aspects, the piggyBac LE ITR sequence is a minimal piggyBac LE ITR sequence. In some aspects, the minimal piggyBac LE ITR is a 35bp piggyBac LE ITR sequence comprising SEQ ID NO: 11.

In some embodiments, the minimal ITR is an LE minimal ITR comprising the sequence CCCTAGAAAGATAGTCTGCGTAAAATTGACGCATG (SEQ ID NO: 14).

In some aspects, the piggyBac LE ITR sequence is a superminimal piggyBac LE ITR sequence. In some aspects, the DNA transposon comprises a second piggyBac ITR sequence, wherein the second piggyBac sequence is a piggyBac right end (RE) ITR sequence. In some aspects, the piggyBac RE ITR sequence is a minimal piggyBac RE ITR sequence. In some aspects, the minimal piggyBac RE ITR is a 63bp piggyBac RE ITR sequence comprising SEQ ID NO: 12. In some embodiments, the minimal ITR is an RE minimal ITR comprising the sequence CCCTAGAAAGATAATCATATTGTGACGTACGTTAAAGATAATCATGCGTAAAATTGA CGCATG (SEQ ID NO: 15). In some embodiments, the piggyBac LE ITR sequence is a superminimal piggyBac LE ITR sequence comprising the sequence:

| (SEQ ID NO: 16) | |
|---|---|
| | CCCTAGAAAGATAATCATATTGTGACGTACGTTAAAGATAATCA; |
| (SEQ ID NO: 17) | |
| | CCCTAGAAAGATAATCATATTGTGACGTACGTTAAAGAT; |
| (SEQ ID NO: 18) | |
| | CCCTAGAAAGATAATCATATTGTGACGTACGTTA; |
| (SEQ ID NO: 19) | |
| | CCCTAGAAAGATAATCATATTGTGACGTA; |
| (SEQ ID NO: 20) | |
| | CCCTAGAAAGATAATCATATTGTG; |
| (SEQ ID NO: 21) | |
| | CCCTAGAAAGATAATCATA; |
| (SEQ ID NO: 22) | |
| | CCCTAGAAAGATAATCA; |
| (SEQ ID NO: 23) | |
| | CCCTAGAAAGATAATC; |
| (SEQ ID NO: 24) | |
| | CCCTAGAAAGATAATCAT; |
| (SEQ ID NO: 25) | |
| | CCCTAGAAAGATAATCATAT; |
| (SEQ ID NO: 26) | |
| | CCCTAGAAAGATAATCATATT; |
| (SEQ ID NO: 27) | |
| | CCCTAGAAAGATAATCATATTG; |
| (SEQ ID NO: 28) | |
| | CCCTAGAAAGATAATCATATTGT; |
| (SEQ ID NO: 29) | |
| | CCCTAGAAAGATAATCATATTGTGA; |
| (SEQ ID NO: 30) | |
| | CCCTAGAAAGATAATCATATTGTGAC; |
| (SEQ ID NO: 31) | |
| | CCCTAGAAAGATAATCATATTGTGACG; |
| (SEQ ID NO: 32) | |
| | CCCTAGAAAGATAATCATATTGTGACGT; |
| (SEQ ID NO: 33) | |
| | CCCTAGAAAGATAATCATATTGTGACGTAC; |
| (SEQ ID NO: 34) | |
| | CCCTAGAAAGATAATCATATTGTGACGTACG; |
| (SEQ ID NO: 35) | |
| | CCCTAGAAAGATAATCATATTGTGACGTACGT; |
| (SEQ ID NO: 36) | |
| | CCCTAGAAAGATAATCATATTGTGACGTACGTT; |
| (SEQ ID NO: 37) | |
| | CCCTAGAAAGATAATCATATTGTGACGTACGTTAA; |
| (SEQ ID NO: 38) | |
| | CCCTAGAAAGATAATCATATTGTGACGTACGTTAAA; |
| (SEQ ID NO: 39) | |
| | CCCTAGAAAGATAATCATATTGTGACGTACGTTAAAG; |
| (SEQ ID NO: 40) | |
| | CCCTAGAAAGATAATCATATTGTGACGTACGTTAAAGA; |
| (SEQ ID NO: 41) | |
| | CCCTAGAAAGATAATCATATTGTGACGTACGTTAAAGATA; |
| (SEQ ID NO: 42) | |
| | CCCTAGAAAGATAATCATATTGTGACGTACGTTAAAGATAA; |
| (SEQ ID NO: 43) | |
| | CCCTAGAAAGATAATCATATTGTGACGTACGTTAAAGATAAT; |
| | or |
| (SEQ ID NO: 44) | |
| | CCCTAGAAAGATAATCATATTGTGACGTACGTTAAAGATAATC. |

In some aspects, the DNA transposon comprises a second piggyBac ITR sequence, wherein the second piggyBac sequence is a piggyBac left end (LE) ITR sequence comprising a 35TCC mutation. The piggyBac ITR sequence comprising the 35TCC mutation has been described in International PCT Application Publication No. WO/2023/060088, which is incorporated herein by reference in its entirety for examples of piggyBac ITR sequences that may be used in the compositions and methods described herein. The 35TCC mutation comprises a G to T substitution at position 31 of the ITR and an A to C substitution at position 33 to create the 35TCC ITR variant. In certain aspects, the 35TCC ITR variant is a full LE ITR sequence comprising SEQ ID NO: 13.

In some aspects, a piggyBac ITR sequence can comprise any piggyBac ITR sequence known in the art. For example, a piggyBac ITR sequence, such as a first piggyBac ITR sequence and/or a second piggyBac ITR sequence in a transposon can comprise, consist essentially of, or consist of a Sleeping Beauty transposon ITR, a Helraiser transposon ITR, a Tol2 transposon ITR, a TcBuster transposon ITR or any combination thereof.

### Lipid Nanoparticles

The present disclosure provides a transposase expressing polynucleotide as described herein encapsulated in a lipid nanoparticle (LNP) for in vivo delivery of the transposase expressing polynucleotide. In some embodiments, the LNP composition comprises at least one cationic lipid and at least one transposase encoding polynucleotide molecule. In some aspects, a lipid nanoparticle can further comprise at least one structural lipid. In some aspects, a lipid nanoparticle can further comprise at least one phospholipid. In some aspects, a lipid nanoparticle can further comprise at least one PEGylated lipid.

Accordingly, the present disclosure provides LNP compositions comprising at least one lipid nanoparticle, wherein the at least one lipid nanoparticle comprises at least one cationic lipid, at least one nucleic acid molecule, at least one structural lipid, at least one phospholipid and at least one PEGylated lipid.

### Bioreducible Ionizable Cationic Lipids

In some aspects, a cationic lipid can be a bioreducible ionizable cationic lipid. Accordingly, the present disclosure provides compositions comprising at least one lipid nanoparticle, wherein the at least one lipid nanoparticle comprises at least one bioreducible ionizable cationic lipid.

In some aspects of the compositions and methods of the present disclosure, a bioreducible ionizable cationic lipid for use in the LNP compositions can be C12-200 (1,1′-[[2-[4-[2-[[2-[bis (2-hydroxydodecyl)amino]ethyl](2-hydroxydodecyl)amino]ethyl]-1-piperazinyl]ethyl]imino]bis-2-dodecanol). In some embodiments, an illustrative LNP composition comprising C12-200 for encapsulating a DNA molecule is: 35% C12-200; 41.84% DOPE; 20% CHOL; and 3.16% DM-PEG. In some embodiments, an illustrative LNP composition comprising C12-200 for encapsulating a RNA molecule is: 33.5% C12-200; 33.5% DOPE; 32% CHOL; and 1% DM-PEG.

In some aspects of the compositions and methods of the present disclosure, the bioionizable cationic lipid is a terpene lipidoid having the structure:

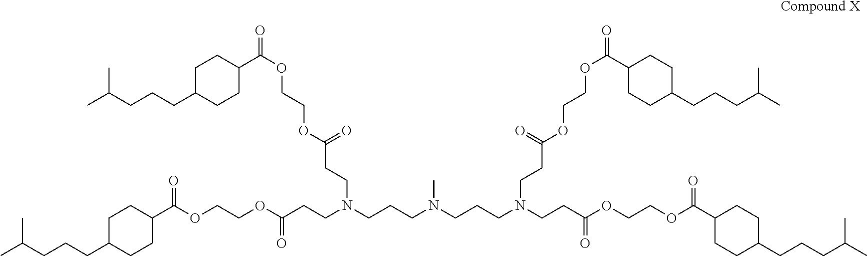 [SMILES: "Compound X" ; CC(C)CCCC1CCC(C(=O)OCCOC(=O)CCN(CCCN(C)CCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]

Methods for preparing Compound X and the preparation of LNP compositions comprising Compound X have been previously described in co-owned International PCT Application No. PCT/US2023/61005. In some embodiments, illustrative LNP compositions comprising Compound X for encapsulating a nucleic molecule are: 41.84% Compound X; 45.85% DOPE; 10% CHOL; and 2.7% DM-PEG or 33.5% Compound X; 32% DOPE; 33.5% CHOL; and 1% DM-PEG.

In some aspects of the compositions and methods of the present disclosure, a bioreducible ionizable cationic lipid for use in the LNP compositions can be ssPalmO-Ph-P4C2. As would be appreciated by the skilled artisan, ssPalmO-Ph-P4C2 has the following structure:

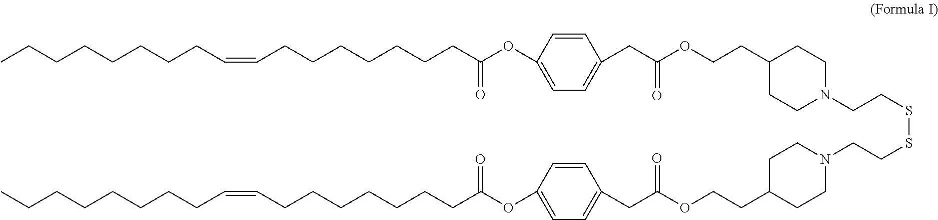 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

As would be appreciated by the skilled artisan, ssPalmO-Ph-P4C2 can also be referred to as Coatsome® SS-OP, ssPalmO-Phe-P4C2, ssPalmO-Phenyl-P4C2, ssPalmO-Phe and ssPalmO-Ph. Accordingly, ssPalmO-Ph-P4C2, Coatsome® SS-OP, ssPalmO-Phe-P4C2, ssPalmO-Phenyl-P4C2, ssPalmO-Phe and ssPalmO-Ph are used interchangeably herein to refer to the bioreducible ionizable cationic lipid with the chemical structure put forth in Formula I.

Without wishing to be bound by theory, three specific segments of ssPalmO-Ph-P4C2 facilitate its biodegradation. First, the tertiary amine of each piperdine ring is an acidic pH-responsive cation-charging unit. Upon endocytosis, the tertiary amine moieties become positively charged in response to the acidic, intracellular endosomal compartment. These are now able to interact and destabilize the membrane and this leads to endosomal escape. Once in the cytosol, the disulfide bond is susceptible to reduction by glutathione generating two free sulfhydryl groups. The resulting high concentration of free thiols further leads to nucleophilic reaction and the particle undergoes self-degradation/collapse via thioesterification and releases the payload in the cytosol. This is defined as HyPER or Hydrolysis accelerated by the intra-Particle Enrichment of Reactant and potentially eliminates the potentially toxic side effects of cationic lipids in general.

As used herein, the term “bioreducible ionizable cationic lipid” is used in its broadest sense to refer to a cationic lipid comprising: at least one tertiary amine, at least one disulfide group, at least one group comprising a bond that is susceptible to cleavage by thioesterification, and further comprising at least two saturated or unsaturated hydrocarbon chains. Illustrative bioreducible ionizable cationic lipids include, but are not limited to, those described in Akita et al., (2020) Biol. Phar. Bull. 43:1617-1625, the contents of which is incorporated herein by reference in their entirety.

In some aspects, a bioreducible ionizable cationic lipid can comprise at least two tertiary amines. In some aspects, at least one tertiary amine cane be a substituted piperidinyl group. In some aspects, each tertiary amine can be a substituted piperidinyl group. In some aspects, a bioreducible ionizable cationic lipid can comprise at least one disulfide bond. In some aspects, the sulfur atoms of the disulfide bond are linked to the nitrogen of the piperdinyl ring via an alkylene group, thereby forming two tertiary amine groups flanking the disulfide bond. In some aspects, at least one of the alkylene groups is an ethylene group. In some aspects, each of the alkylene groups is an ethylene group.

In some aspects, an at least one group comprising a bond that is susceptible to cleavage by thioesterification can be a phenyl ester group. In some aspects, a bioreducible ionizable cationic lipid can comprise at least two phenyl ester groups. In some aspects, at least one of the at least two saturated or unsaturated hydrocarbon chains is an unsaturated hydrocarbon chain. In some aspects, each of the least two saturated or unsaturated hydrocarbon chains is an unsaturated hydrocarbon chain. In some aspects, an unsaturated hydrocarbon chain can be an octadecene. In some aspects, an octadecene can be (Z)-octadec-9-ene. In some aspects, an (Z)-octadec-9-ene group can linked to a phenyl ester group of the bioreducible ionizable cationic lipid.

Illustrative bioreducible ionizable cationic lipids and methods of preparing such lipids useful in the methods of the present invention include those disclosed in International Patent Application No. PCT/JP2016/052690, published as WO/2016/121942, the contents of which are incorporated herein by reference in their entirety for examples of bioreducible ionizable cationic lipids that may be used in the compositions and methods described herein. Accordingly, the present disclosure provides compositions comprising at least one lipid nanoparticle, wherein the at least one lipid nanoparticle comprises any one of the bioreducible ionizable cationic lipids put forth in WO/2016/121942.

Accordingly, the present disclosure provides compositions comprising at least one lipid nanoparticle, wherein the at least one lipid nanoparticle comprises at least one bioreducible ionizable cationic lipid, at least one nucleic acid molecule, at least one structural lipid, at least one phospholipid and at least one PEGylated lipid.

In some aspects, the bioreducible ionizable cationic lipid can be ssPalmO-Ph-P4C2, having the structure put forth in Formula I (see Akita et al., (2020) Biol. Phar. Bull. 43:1617-1625, the contents of which are incorporated by reference in their entirety).

As described herein, the LNP compositions of the present disclosure that comprise at least one bioreducible ionizable cationic lipid advantageously exhibit significantly reduced toxicity in animals as compared to LNP compositions comprising non-bioreducible ionizable cationic lipids. In particular, administration the LNP compositions of the present disclosure surprisingly does not result in any body weight loss. In fact, the LNP compositions of the present disclosure are so non-toxic that animals administered the LNPs actually gain body weight, even when administered amounts of LNPs that exceed the lethal dose of LNP compositions comprising non-bioreducible ionizable cationic lipids.

### LNP Components

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one bioreducible ionizable cationic lipid by moles.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one bioreducible ionizable cationic lipid by moles.

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5% or about 60%, or about 62.5%, or about 65%, or about 67.5%, or about 70% of at least one structural lipid by moles.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5% or at least about 60%, or at least about 62.5%, or at least about 65%, or at least about 67.5%, or at least about 70% of at least one structural lipid by moles.

In some aspects, an LNP of the present disclosure can comprise about 2.5%, or about 5%, or about 7.5%, or about 10%, or about 12.5%, or about 15%, or about 17.5%, or about 20%, or about 22.5%, or about 25%, or about 27.5%, or about 30%, or about 32.5%, or about 35%, or about 37.5%, or about 40%, or about 42.5%, or about 45%, or about 47.5%, or about 50%, or about 52.5%, or about 55%, or about 57.5%, or about 60%, or about 62.5%, or about 65%, or about 67.5%, or about 70% of at least one phospholipid by moles.

In some aspects, an LNP of the present disclosure can comprise at least about 2.5%, or at least about 5%, or at least about 7.5%, or at least about 10%, or at least about 12.5%, or at least about 15%, or at least about 17.5%, or at least about 20%, or at least about 22.5%, or at least about 25%, or at least about 27.5%, or at least about 30%, or at least about 32.5%, or at least about 35%, or at least about 37.5%, or at least about 40%, or at least about 42.5%, or at least about 45%, or at least about 47.5%, or at least about 50%, or at least about 52.5%, or at least about 55%, or at least about 57.5%, or at least about 60%, or at least about 62.5%, or at least about 65%, or at least about 67.5%, or at least about 70% of at least one phospholipid by moles.

In some aspects, an LNP of the present disclosure can comprise about 0.25%, or about 0.5%, or about 0.75%, or about 1.0%, or about 1.25%, or about 1.5%, or about 1.75%, or about 2.0%, or at least about or about 2.5%, or about 5% of at least one PEGylated lipid by moles.

In some aspects, an LNP of the present disclosure can comprise at least about 0.25%, or at least about 0.5%, or at least about 0.75%, or at least about 1.0%, or at least about 1.25%, or at least about 1.5%, or at least about 1.75%, or at least about 2.0%, or at least about or at least about 2.5%, or at least about 5% of at least one PEGylated lipid by moles.

### Structural Lipids

In some aspects, a structural lipid can be a steroid. In some aspects, a structural lipid can be a sterol. In some aspects, a structural lipid can comprise cholesterol. In some aspects, a structural lipid can comprise ergosterol. In some aspects, a structural lipid can be a phytosterol.

### Phospholipid

As used herein, the term “phospholipid” is used in its broadest sent to refer to any amphiphilic molecule that comprises a polar (hydrophilic) headgroup comprising phosphate and two hydrophobic fatty acid chains. In some aspects, a phospholipid can comprise dioleoylphosphatidylethanolamine (DOPE). In some aspects, a phospholipid can comprise DDPC (1,2-Didecanoyl-sn-glycero-3-phosphocholine), DEPA-NA (1,2-Dierucoyl-sn-glycero-3-phosphate (Sodium Salt)), DEPC (1,2-Dierucoyl-sn-glycero-3-phosphocholine), DEPE (1,2-Dierucoyl-sn-glycero-3-phosphoethanolamine), DEPG-NA (1,2-Dierucoyl-sn-glycero-3 [Phospho-rac-(1-glycerol) (Sodium Salt)), DLOPC (1,2-Dilinoleoyl-sn-glycero-3-phosphocholine), DLPA-NA (1,2-Dilauroyl-sn-glycero-3-phosphate (Sodium Salt)), DLPC (1,2-Dilauroyl-sn-glycero-3-phosphocholine), DLPE (1,2-Dilauroyl-sn-glycero-3-phosphoethanolamine), DLPG-NA (1,2-Dilauroyl-sn-glycero-3 [Phospho-rac-(1-glycerol) (Sodium Salt)), DLPG-NH4 (1,2-Dilauroyl-sn-glycero-3 [Phospho-rac-(1-glycerol) (Ammonium Salt)), DLPS-NA (1,2-Dilauroyl-sn-glycero-3-phosphoserine (Sodium Salt)), DMPA-NA (1,2-Dimyristoyl-sn-glycero-3-phosphate (Sodium Salt)), DMPC (1,2-Dimyristoyl-sn-glycero-3-phosphocholine), DMPE (1,2-Dimyristoyl-sn-glycero-3-phosphoethanolamine), DMPG-NA (1,2-Dimyristoyl-sn-glycero-3 [Phospho-rac-(1-glycerol) (Sodium Salt)), DMPG-NH4 (1,2-Dimyristoyl-sn-glycero-3 [Phospho-rac-(1-glycerol) (Ammonium Salt)), DMPG-NH4/NA (1,2-Dimyristoyl-sn-glycero-3 [Phospho-rac-(1-glycerol) (Sodium/Ammonium Salt)), DMPS-NA (1,2-Dimyristoyl-sn-glycero-3-phosphoserine (Sodium Salt)), DOPA-NA (1,2-Dioleoyl-sn-glycero-3-phosphate (Sodium Salt)), DOPC (1,2-Dioleoyl-sn-glycero-3-phosphocholine), DOPE (1,2-Dioleoyl-sn-glycero-3-phosphoethanolamine), DOPG-NA (1,2-Dioleoyl-sn-glycero-3 [Phospho-rac-(1-glycerol) (Sodium Salt)), DOPS-NA (1,2-Dioleoyl-sn-glycero-3-phosphoserine (Sodium Salt)), DPPA-NA (1,2-Dipalmitoyl-sn-glycero-3-phosphate (Sodium Salt)), DPPC (1,2-Dipalmitoyl-sn-glycero-3-phosphocholine), DPPE (1,2-Dipalmitoyl-sn-glycero-3-phosphoethanolamine), DPPG-NA (1,2-Dipalmitoyl-sn-glycero-3 [Phospho-rac-(1-glycerol) (Sodium Salt)), DPPG-NH4 (1,2-Dipalmitoyl-sn-glycero-3 [Phospho-rac-(1-glycerol) (Ammonium Salt)), DPPS-NA (1,2-Dipalmitoyl-sn-glycero-3-phosphoserine (Sodium Salt)), DSPA-NA (1,2-Distearoyl-sn-glycero-3-phosphate (Sodium Salt)), DSPC (1,2-Distearoyl-sn-glycero-3-phosphocholine), DSPE (1,2-Distearoyl-sn-glycero-3-phosphoethanolamine), DSPG-NA (1,2-Distearoyl-sn-glycero-3 [Phospho-rac-(1-glycerol) (Sodium Salt)), DSPG-NH4 (1,2-Distearoyl-sn-glycero-3 [Phospho-rac-(1-glycerol) (Ammonium Salt)), DSPS-NA (1,2-Distearoyl-sn-glycero-3-phosphoserine (Sodium Salt)), EPC (Egg-PC), HEPC (Hydrogenated Egg PC), HSPC (Hydrogenated Soy PC), LYSOPC MYRISTIC (1-Myristoyl-sn-glycero-3-phosphocholine), LYSOPC PALMITIC (1-Palmitoyl-sn-glycero-3-phosphocholine), LYSOPC STEARIC (1-Stearoyl-sn-glycero-3-phosphocholine), Milk Sphingomyelin (MPPC; 1-Myristoyl-2-palmitoyl-sn-glycero 3-phosphocholine), MSPC (1-Myristoyl-2-stearoyl-sn-glycero-3-phosphocholine), PMPC (1-Palmitoyl-2-myristoyl-sn-glycero-3-phosphocholine), POPC (1-Palmitoyl-2-oleoyl-sn-glycero-3-phosphocholine), POPE (1-Palmitoyl-2-oleoyl-sn-glycero-3-phosphoethanolamine), POPG-NA (1-Palmitoyl-2-oleoyl-sn-glycero-3 [Phospho-rac-(1-glycerol)] (Sodium Salt)), PSPC (1-Palmitoyl-2-stearoyl-sn-glycero-3-phosphocholine), SMPC (1-Stearoyl-2-myristoyl-sn-glycero-3-phosphocholine), SOPC (1-Stearoyl-2-oleoyl-sn-glycero-3-phosphocholine), SPPC (1-Stearoyl-2-palmitoyl-sn-glycero-3-phosphocholine), or any combination thereof.

### PEGylated Lipid

As used herein, the term “PEGylated lipid” is used to refer to any lipid that is modified (e.g. covalently linked to) at least one polyethylene glycol molecule. In some aspects, a PEGylated lipid can comprise 1,2-dimyristoyl-rac-glycero-3-methoxypolyethylene glycol-2000, hereafter referred to as DMG-PEG2000.

### Nucleic Acids

In some aspects, a lipid nanoparticle can comprise at least one transposase expressing polynucleotide molecule. In some aspects, a lipid nanoparticle can comprise a plurality of transposase expressing polynucleotide molecules. In some aspects, the at least one transposase expressing polynucleotide molecule or the plurality of transposase expressing polynucleotide molecules can be formulated in a lipid nanoparticle.

In some aspects, a nucleic acid molecule can be a synthetic transposase expressing polynucleotide molecule. In some aspects, a transposase expressing polynucleotide molecule can be a non-naturally occurring nucleic acid molecule. In some aspects, a non-naturally occurring nucleic acid molecule can comprise at least one non-naturally occurring nucleotide. The at least one non-naturally occurring nucleotide can be any non-naturally occurring nucleotide known in the art. In some aspects, a nucleic acid molecule can be a modified transposase expressing polynucleotide molecule. In some aspects, a modified nucleic acid molecule can comprise at least one modified nucleotide. The at least one modified nucleotide can be any modified nucleic acid known in the art.

In some aspects, a lipid nanoparticle can comprise lipid and nucleic acid at a specified ratio (weight/weight).

In some aspects, a lipid nanoparticle comprising at least one nucleic acid can comprise lipid and nucleic acid at a ratio of about 5:1 to about 15:1, or about 10:1 to about 20:1, or about 15:1 to about 25:1, or about 20:1 to about 30:1, or about 25:1 to about 35:1 or about 30:1 to about 40:1, or about 35:1 to about 45:1, or about 40:1 to about 50:1, or about 45:1 to about 55:1, or about 50:1 to about 60:1, or about 55:1 to about 65:1, or about 60:1 to about 70:1, or about 65:1 to about 75:1, or about 70:1 to about 80:1, or about 75:1 to about 85:1, or about 80:1 to about 90:1, or about 85:1 to about 95:1, or about 90:1 to about 100:1, or about 95:1 to about 105:1, or about 100:1 to about 110:1, or about 105:1 to about 115:1, or about 110:1 to about 120:1, or about 115:1 to about 125:1, or about 120:1 to about 130:1, or about 125:1 to about 135:1, or about 130:1 to about 140:1, or about 135:1 to about 145:1, or about 140:1 to about 150:1, lipid: nucleic acid, weight/weight.

In some aspects, a lipid nanoparticle can comprise lipid and nucleic acid at a ratio of about 5:1, or about 10:1, or about 15:1, or about 20:1, or about 25:1, or about 30:1, or about 35:1, or about 40:1, or about 45:1, or about 50:1, or about 55:1, or about 60:1, or about 65:1, or about 70:1, or about 75:1, or about 80:1, or about 85:1, or about 90:1, or about 95:1, or about 100:1, or about 105:1, or about 110:1, or about 115:1, or about 120:1, or about 125:1, or about 130:1, or about 135:1, or about 140:1, or about 145:1, or about 150:1, or about 200:1, lipid: nucleic acid, weight/weight.

In some aspects, a lipid nanoparticle can comprise lipid and nucleic acid at a ratio of about 10:1, or about 17.5:1, or about 25:1, lipid: nucleic acid, weight/weight.

In some aspects, a nucleic acid molecule can be an RNA molecule. Thus, in some aspects, a lipid nanoparticle can comprise at least one transposase expressing polynucleotide, wherein the polynucleotide molecule is an RNA molecule. In some aspects, an RNA molecule can be an mRNA molecule. In some aspects, an mRNA molecule can comprise a 5′-CAP.

In some aspects, an mRNA molecule can be capped using any method and/or capping moiety known in the art. An mRNA molecule can be capped with m7G (5′) ppp (5′) G moiety. A m7G (5′) ppp (5′) G moiety is also referred to herein as a “Cap0”. An mRNA molecule can be capped with a CleanCap® moiety. A CleanCap® moiety can comprise a m7G (5′) ppp (5′) (2′OMeA) (CleanCap® AG) moiety. A CleanCap® moiety can comprise a m7G (5′) ppp (5′) (2′OMeG) (CleanCap® GG) moiety. An mRNA molecule can be capped with an anti-reverse cap analog (ARCA®) moiety. An ARCA® moiety can comprise a m7 (3′-O-methyl) G (5′) ppp (5′) G moiety. An mRNA molecule can be capped with a CleanCap® 3′OMe moiety (CleanCap®+ARCA®).

In some aspects, the mRNA molecule comprising a transposase expressing polynucleotide as described herein is prepared according to the method in Example 10.

In some aspects, an mRNA molecule can comprise at least one modified nucleic acid.

Modified nucleic acids can include, but are not limited to, 5-methoxyuridine (5moU), N₁-methylpseudouridine (me¹Ψ), pseudouridine (Ψ), 5-methylcytidine (5-MeC).

### DNA Editing Compositions

The present disclosure also provides gene editing compositions and cells comprising the gene editing compositions. The gene editing composition can comprise a nucleic acid sequence encoding a DNA binding domain and a nucleic acid sequence encoding a nuclease protein or a nuclease domain thereof. The sequence encoding a nuclease protein or the sequence encoding a nuclease domain thereof can comprise a DNA sequence, an RNA sequence, or a combination thereof. The nuclease or the nuclease domain thereof can comprise one or more of a CRISPR/Cas protein, a Transcription Activator-Like Effector Nuclease (TALEN), a Zinc Finger Nuclease (ZFN), and an endonuclease.

The nuclease or the nuclease domain thereof can comprise a nuclease-inactivated Cas (dCas) protein and an endonuclease. The endonuclease can comprise a Clo051 nuclease or a nuclease domain thereof. The gene editing composition can comprise a fusion protein. In some embodiments, the fusion protein comprises a nuclease-inactivated Cas9 (dCas9) protein and a Clo051 nuclease or a Clo051 nuclease domain. The gene editing composition can further comprise a guide sequence. The guide sequence comprises an RNA sequence.

The disclosure provides compositions comprising a Cas9 operatively-linked to an effector. The disclosure provides a fusion protein comprising, consisting essentially of or consisting of a DNA localization component and an effector molecule, wherein the effector comprises a Cas9. A Cas9 construct of the disclosure can comprise an effector comprising a type IIS endonuclease.

In some embodiments, a gene editing composition comprises an inactivated, Cas9 (dSaCas9) operatively-linked to an effector. The disclosure provides a fusion protein comprising a DNA localization component and an effector molecule, wherein the effector comprises an inactivated Cas9 (dSaCas9). An inactivated Cas9 (dSaCas9) construct of the disclosure can comprise an effector comprising a type IIS endonuclease. A dSaCas9 comprises the amino acid sequence of SEQ ID NO: 48, which includes a D10A and a N580A mutation to inactivate the catalytic site.

The disclosure provides compositions comprising an inactivated Cas9 (dCas9) operatively-linked to an effector. The disclosure further provides a fusion protein comprising a DNA localization component and an effector molecule, wherein the effector comprises an inactivated Cas9 (dCas9). An inactivated Cas9 (dCas9) construct of the disclosure can comprise an effector comprising a type IIS endonuclease.

The dCas9 can be isolated or derived from Streptococcus pyogenes. The dCas9 can comprise a dCas9 with substitutions at amino acid positions 10 and 840, which inactivate the catalytic site. In some aspects, these substitutions are D10A and H840A. The dCas9 can comprise the amino acid sequence of SEQ ID NO: 49 or SEQ ID NO: 50.

In some embodiments, the C-terminus of the dCas9 or inactivated nuclease domain thereof, is joined to N-terminus of the Clo051 polypeptide or nuclease domain thereof via peptide linker sequence selected from GGGGS (SEQ ID NO: 60).

In some embodiments, the Clo051 nuclease domain comprises the amino acid sequence of SEQ ID NO: 51. In some embodiments, the Clo051 nuclease domain comprises at least one amino acid substitution relative to SEQ ID NO: 51. In some aspects, the amino acid substitution is in the alpha-helix-loop domain of the Clo051 nuclease. In some aspects, the amino acid substitution is at position 35, 37, 60, 92, 98, 100 or 146 of SEQ ID NO: 51. In some aspects, the amino acid substitution is at position 37 of SEQ ID NO: 51. In some aspects, the amino acid substitution is at positions 37 and 92 of SEQ ID NO: 51.

An illustrative dCas9-Clo051 (Cas-CLOVER) fusion protein can comprise the amino acid sequence of SEQ ID NO: 52. The illustrative dCas9-Clo051 fusion protein can be encoded by a polynucleotide which comprises the nucleic acid sequence of SEQ ID NO: 53. The nucleic acid encoding the dCas9-Clo051 fusion protein can be DNA or RNA.

An illustrative dCas9-Clo051 (Cas-CLOVER) fusion protein can comprise the amino acid sequence of SEQ ID NO: 54. The illustrative dCas9-Clo051 fusion protein can be encoded by a polynucleotide which comprises the nucleic acid sequence of SEQ ID NO: 55. The nucleic acid encoding the dCas9-Clo051 fusion protein can be DNA or RNA.

An illustrative dCas9-Clo051 fusion (Cas-CLOVER) fusion protein of the disclosure may further comprise at least one nuclear localization sequence (NLS). In some embodiments, the dCas9-Clo051 fusion protein of the disclosure comprises two nuclear localization sequences. In some embodiments, the NLS is on the N-terminal end of the dCas9-Clo051 fusion protein (NLS-dCas9-Clo051). In some embodiments, the NLS is on the C-terminal end of the dCas9-Clo051 fusion protein (dCas9-Clo051-NLS). In some embodiments, the NLS is on the N-terminal end and at the C-terminal end of the dCas9-Clo051 fusion protein (“NLS-dCas9-Clo051-NLS” or “wildtype Cas-CLOVER” or “dspCas9 Ca-CLOVER”).

The NLS-dCas9-Clo051-NLS (“wildtype Cas-CLOVER”, or “Cas-CLOVER v2”, or “CCv2”, or “dspCas9 Cas-CLOVER”) fusion protein can comprise the amino acid sequence of SEQ ID NO: 56, where the NLS amino acid sequence is bolded and underlined and the linker is bolded and italicized):

(SEQ ID NO: 56)
MAPKKKRKVEGIKSNISLLKDELRGQISHISHEYLSLIDLAFDSKQNRLF
EMKVLELLVNEYGFKGRHLGGSRKPDGIVYSTTLEDNFGIIVDTKAYSEG
YSLPISQADEMERYVRENSNRDEEVNPNKWWENFSEEVKKYYFVFISGSF
KGKFEEQLRRLSMTTGVNGSAVNVVNLLLGAEKIRSGEMTIEELERAMEN
NSEFILKYGGGGSDKKYSIGLAIGTNSVGWAVITDEYKVPSKKFKVLGNT
DRHSIKKNLIGALLEDSGETAEATRLKRTARRRYTRRKNRICYLQEIFSN
EMAKVDDSFFHRLEESELVEEDKKHERHPIFGNIVDEVAYHEKYPTIYHL
RKKLVDSTDKADLRLIYLALAHMIKERGHFLIEGDLNPDNSDVDKLFIQL
VQTYNQLFEENPINASGVDAKAILSARLSKSRRLENLIAQLPGEKKNGLF
GNLIALSLGLTPNEKSNEDLAEDAKLQLSKDTYDDDLDNLLAQIGDQYAD
LFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEE
LLVKLNREDLLRKQRTEDNGSIPHQIHLGELHAILRRQEDFYPELKDNRE
KIEKILTFRIPYYVGPLARGNSRFAWMTRKSEETITPWNFEEVVDKGASA
QSFIERMTNEDKNLPNEKVLPKHSLLYEYFTVYNELTKVKYVTEGMRKPA
FLSGEQKKAIVDLLEKTNRKVTVKQLKEDYFKKIECFDSVEISGVEDREN
ASLGTYHDLLKIIKDKDELDNEENEDILEDIVLTLTLFEDREMIEERLKT
YAHLEDDKVMKQLKRRRYTGWGRLSRKLINGIRDKQSGKTILDELKSDGF
ANRNEMQLIHDDSLTFKEDIQKAQVSGQGDSLHEHIANLAGSPAIKKGIL
QTVKVVDELVKVMGRHKPENIVIEMARENQTTQKGQKNSRERMKRIEEGI
KELGSQILKEHPVENTQLQNEKLYLYYLQNGRDMYVDQELDINRLSDYDV
DAIVPQSFLKDDSIDNKVLTRSDKNRGKSDNVPSEEVVKKMKNYWRQLLN
AKLITQRKEDNLTKAERGGLSELDKAGFIKRQLVETRQITKHVAQILDSR
MNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPT
VAYSVLVVAKVEKGKSKKLKSVKELLGITIMERSSFEKNPIDELEAKGYK
EVKKDLIIKLPKYSLFELENGRKRMLASAGELQKGNELALPSKYVNFLYL
ASHYEKLKGSPEDNEQKQLEVEQHKHYLDEIIEQISEFSKRVILADANLD
KVLSAYNKHRDKPIREQAENIIHLFTLTNLGAPAAFKYEDTTIDRKRYTS
TKEVLDATLIHQSITGLYETRIDLSQLGGDGSPKKKRKVSS.

In some embodiments, the NLS-dCas9-Clo051-NLS (“wildtype Cas-CLOVER”, or “Cas-CLOVER v2”, or “CCv2”, or “dspCas9 Cas-CLOVER”) fusion protein comprises an amino acid sequence that is at least 70%, at least 75%, at least 80%, at least 85%, at least 90%, at least 95%, at least 96%, at least 97%, at least 98% or at least 99% identical to the amino acid sequence set forth in SEQ ID NO: 56. In some embodiments, the fusion protein comprises an amino acid sequence that is at least 70%, at least 75%, at least 80%, at least 85%, at least 90%, at least 95%, at least 96%, at least 97%, at least 98% or at least 99% identical to the amino acid sequence set forth in SEQ ID NO: 56.

In some embodiments, the NLS-dCas9-Clo051-NLS (“wildtype Cas-CLOVER”, or “Cas-CLOVER v2”, or “CCv2”, or “dspCas9 Cas-CLOVER”) fusion protein comprises the amino acid sequence set forth in SEQ ID NO: 56 with one, two, three, four or five conservative amino acid substitutions.

The nucleic acid encoding the NLS-dCas9-Clo051-NLS (“wildtype Cas-CLOVER”, or “Cas-CLOVER v2”, or “CCv2”, or “dspCas9 Cas-CLOVER”) fusion protein can be DNA or RNA. In some embodiments, a dCas9-Clo051 fusion protein comprising two NLS regions is encoded by an mRNA sequence comprising the sequence set forth in SEQ ID NO: 57 or a DNA sequence comprising the sequence set forth in SEQ ID NO: 61.

(SEQ ID NO: 61)
atggctcccaagaagaagcggaaggtcGAGGGCATCAAGAGCAACATCAGCCTGCTGAAGGACGAGCTGA
GAGGCCAGATCAGCCACATCTCCCACGAGTACCTGAGCCTGATCGACCTGGCCTTCGACCCCAAGCAGAA
CCGGCTGTTCGAGATGAAGGTGCTGGAACTGCTGGTCAACGAGTACGGCTTCAAGGGCAGACACCTCGGC
GGCAGCAGAAAGCCTGATGGCATCGTGTACAGCACCACACTCGAGGACAACTTCGGCATCATCGTGGACA
CCAAGGCCTACAGCGAGGGCTACAGCCTGCCTATCTCTCAGGCCGACGAGATGGAAAGATACGTGCGCGA
GAACAGCAACCGCGACGAGGAAGTGAACCCCAACAAGTGGTGGGAGAACTTCAGCGAGGAAGTCAAAAAG
TACTACTTCGTGTTCATCAGCGGCAGCTTTAAGGGCAAGTTCGAGGAACAGCTGCGGCGGCTGTCTATGA
CCACAGGCGTTAACGGCAGCGCCGTGAACGTGGTCAATCTGCTGCTGGGCGCCGAGAAGATTAGAAGCGG
CGAGATGACCATCGAGGAACTGGAACGGGCCATGTTCAACAACAGCGAGTTCATCCTGAAGTACggcgga
ggcggcagcgacaagaagtactctatcggactggccatcggcaccaactctgttggatgggccgtgatca
ccgacgagtacaaggtgcccagcaagaaattcaaagtgctgggcaacaccgaccggcacagcatcaagaa
gaatctgatcggcgccctgctgttcgactctggcgaaacagccgaagccaccagactgaagagaaccgcc
agacggcggtacaccagaagaaagaaccggatctgctacctgcaagagatcttcagcaacgagatggcca
aggtggacgacagcttcttccacagactggaagagtccttcctggtggaagaggacaagaagcacgagcg
gcaccccatcttcggaaatatcgtggacgaggtggcctaccacgagaagtaccccaccatctaccacctg
agaaagaaactggtggacagcaccgacaaggccgacctgcgactgatctatctggccctggctcacatga
tcaagttccggggccacttcctgatcgagggcgacctgaatcctgacaactccgacgtggacaagctgtt
catccagctggtgcagacctacaatcagctgttcgaagagaatcccatcaacgcctctggcgtggacgcc
aaagccatcctgtctgccagactgagcaagagcagacggctggaaaacctgatcgctcagctgcccggcg
agaagaagaatggcctgttcggcaacctgattgccctgtctctgggcctgacacctaacttcaagtccaa
cttcgatctggccgaggatgccaaactgcagctgtccaaggacacctacgacgacgacctggataacctg
ctggcccagatcggcgatcagtacgccgacttgtttctggccgccaagaacctgtctgacgccatcctgc
tgagcgacatcctgagagtgaacaccgagatcacaaaggcccctctgagcgcctctatgatcaagagata
cgacgagcaccaccaggatctgaccctgctgaaagctctcgtcaggcagcagctgccagagaagtacaaa
gagattttcttcgaccagagcaagaacggctacgccggctacattgatggcggagccagccaagaggaat
tctacaagttcatcaagcccatcctcgagaagatggacggcacagaggaactgctcgtgaagctgaacag
agaggacctgctgcggaagcagcggaccttcgacaatggctctatccctcaccagatccacctgggagag
ctgcacgccattctgcggagacaagaggacttttacccattcctgaaggacaaccgggaaaagattgaga
agatcctgaccttcaggatcccctactacgtgggaccactggccagaggcaatagcagattcgcctggat
gaccagaaagagcgaggaaaccatcacaccctggaacttcgaagaggtggtggacaagggcgccagcgct
cagtccttcatcgagcggatgaccaatttcgacaagaatctgcccaacgagaaagtgctgcccaagcact
ccctgctgtacgagtacttcaccgtgtacaacgagctgaccaaagtgaaatacgtgaccgagggaatgag
aaagcccgcctttctgtccggcgagcagaaaaaggccatcgtggatctgctgttcaagaccaaccggaaa
gtgaccgtgaagcagctgaaagaggactacttcaagaaaatcgagtgcttcgactccgtggaaatcagcg
gcgtggaagatcggttcaatgccagcctgggcacataccacgatctgctgaaaattatcaaggacaagga
cttcctggacaacgaggaaaacgaggacatccttgaggacatcgtgctgaccctgacactgttcgaggac
agagagatgatcgaggaaaggctgaaaacatacgcccacctgttcgacgacaaagtcatgaagcaactga
agcggcggcgctacacaggctggggcagactgtctagaaagctgatcaacggcatccgggacaagcagtc
cggcaagaccatcctggactttctgaagtccgacggcttcgccaacagaaacttcatgcagctgattcac
gacgacagcctcaccttcaaagaggacattcagaaggcccaggtttccggccagggcgattctctgcacg
agcacattgccaatctggccggctctcccgccattaagaagggcattctgcagacagtgaaagtggtgga
tgagctggtcaaagtgatggggagacacaagcccgagaacatcgtgatcgaaatggccagagagaaccag
accacacagaagggccagaagaactcccgcgagagaatgaagcggatcgaagagggaatcaaagagctgg
ggagccagatcctgaaagaacaccccgtggaaaacacccagctgcagaacgagaagctgtacctgtacta
cctccagaacggccgggatatgtacgtggaccaagagctggacatcaaccgcctgagcgactacgatgtg
gacgctatcgtgccccagtcttttctgaaagatgactccatcgacaacaaggtgctgaccagaagcgata
agaaccggggcaagagcgacaacgtgccctctgaagaggtcgtgaagaagatgaagaactactggcgaca
gctgctgaacgccaagctgattacccagcggaagttcgataacctgaccaaggccgagagaggcggcctg
tctgaactggataaggccggcttcatcaagagacagctggtggaaacccggcagatcaccaaacacgtgg
cacagattctggactcccggatgaacaccaaatacgatgagaacgacaaactgatccgggaagtgaaagt
catcaccctgaagtccaagctggtgtccgatttccggaaggatttccagttctacaaagtgcgggaaatc
aacaactaccatcacgcccacgacgcctacctgaatgccgttgttggaacagccctgatcaagaagtatc
ccaagctggaaagcgagttcgtgtacggcgactacaaggtgtacgacgtgcggaagatgatcgccaagag
cgagcaagagattggaaaggctaccgccaaatacttcttctactccaacatcatgaactttttcaagaca
gagatcaccctcgccaacggcgagatcagaaagcggcctctgatcgagacaaacggcgaaaccggcgaga
ttgtgtgggataagggcagagactttgccacagtgcggaaggtgctcagcatgccccaagtgaatatcgt
gaaaaagaccgaggtgcagacaggcggcttcagcaaagagtccattctgcctaagcggaactccgacaag
ctgatcgcccggaagaaggactgggaccccaagaaatacggcggcttcgatagccctaccgtggcctatt
ctgtgctggtggtggccaaagtggaaaagggaaagtccaagaagctcaagagcgtcaaagaactcctggg
catcaccatcatggaacggtccagcttcgagaagaaccctatcgactttctggaagccaagggctacaaa
gaagtcaagaaggacctgatcatcaagctccccaagtacagcctgttcgagctggaaaatggccggaagc
ggatgctggcttctgctggcgaactgcagaagggaaacgaactggccctgcctagcaaatatgtgaactt
cctgtacctggccagccactatgagaagctgaagggcagccccgaggacaatgagcagaagcagcttttc
gtcgagcagcacaagcactacctggacgagatcatcgagcagatctccgagttctccaagagagtgatcc
tggccgacgccaacctggacaaggttctgtccgcctacaacaagcaccgggataagcccatcagagagca
ggccgagaatatcatccacctgtttaccctgaccaacctgggagcccctgccgccttcaagtacttcgac
accaccatcgaccggaagcgctacaccagcaccaaagaagtgctggacgccacactgatccaccagagca
tcaccggcctgtacgagacacggatcgatctgtctcagcttggaggcgacggcagccctaagaagaagag
aaaggtttccagctaataa.

In some aspects, NLS-dCas9-Clo051-NLS (“wildtype Cas-CLOVER”) comprises at least one amino acid substitution relative to SEQ ID NO: 56. In some aspects, the amino acid substitution is located in the Clo051 domain of the NLS-dCas9-Clo051-NLS.

In some aspects, the NLS-dCas9-Clo051-NLS of SEQ ID NO: 56 can comprise at least one substitution at amino acid positions 42, 44, 67, 105, 107 or 153. In some aspects, the amino acid substitutions are F42E, F42D, S44E, S44P, R67E, I105Q, Q107A, Q107E, Q107H, Q107D and/or K153D. In some aspects, the amino acid substitution is S44P.

An illustrative S44P mutant NLS-dCas9-Clo051-NLS (“S44P Cas-CLOVER”, or “S44P CC”, or “S44P”, or “Cas-CLOVERv3”, or “CCv3”) fusion protein can comprise the amino acid sequence of SEQ ID NO: 58, where the NLS amino acid sequence is bolded and underlined and the linker is bolded and italicized:

(SEQ ID NO: 58)
MAPKKKRKVEGIKSNISLLKDELRGQISHISHEYLSLIDLAFDPKQNRLF
EMKVLELLVNEYGFKGRHLGGSRKPDGIVYSTTLEDNFGIIVDTKAYSEG
YSLPISQADEMERYVRENSNRDEEVNPNKWWENFSEEVKKYYFVFISGSF
KGKFEEQLRRLSMTTGVNGSAVNVVNLLLGAEKIRSGEMTIEELERAMEN
NSEFILKYGGGGSDKKYSIGLAIGTNSVGWAVITDEYKVPSKKFKVLGNT
DRHSIKKNLIGALLEDSGETAEATRLKRTARRRYTRRKNRICYLQEIFSN
EMAKVDDSFFHRLEESELVEEDKKHERHPIFGNIVDEVAYHEKYPTIYHL
RKKLVDSTDKADLRLIYLALAHMIKERGHFLIEGDLNPDNSDVDKLFIQL
VQTYNQLFEENPINASGVDAKAILSARLSKSRRLENLIAQLPGEKKNGLF
GNLIALSLGLTPNEKSNEDLAEDAKLQLSKDTYDDDLDNLLAQIGDQYAD
LFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEE
LLVKLNREDLLRKQRTEDNGSIPHQIHLGELHAILRRQEDFYPELKDNRE
KIEKILTFRIPYYVGPLARGNSRFAWMTRKSEETITPWNFEEVVDKGASA
QSFIERMTNEDKNLPNEKVLPKHSLLYEYFTVYNELTKVKYVTEGMRKPA
FLSGEQKKAIVDLLEKTNRKVTVKQLKEDYFKKIECFDSVEISGVEDRFN
ASLGTYHDLLKIIKDKDELDNEENEDILEDIVLTLTLFEDREMIEERLKT
YAHLEDDKVMKQLKRRRYTGWGRLSRKLINGIRDKQSGKTILDELKSDGF
ANRNEMQLIHDDSLTFKEDIQKAQVSGQGDSLHEHIANLAGSPAIKKGIL
QTVKVVDELVKVMGRHKPENIVIEMARENQTTQKGQKNSRERMKRIEEGI
KELGSQILKEHPVENTQLQNEKLYLYYLQNGRDMYVDQELDINRLSDYDV
DAIVPQSFLKDDSIDNKVLTRSDKNRGKSDNVPSEEVVKKMKNYWRQLLN
AKLITQRKEDNLTKAERGGLSELDKAGFIKRQLVETRQITKHVAQILDSR
MNTKYDENDKLIREVKVITLKSKLVSDERKDFQFYKVREINNYHHAHDAY
LNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYL
ASHYEKLKGSPEDNEQKQLEVEQHKHYLDEIIEQISEFSKRVILADANLD
KVLSAYNKHRDKPIREQAENIIHLETLTNLGAPAAFKYEDTTIDRKRYTS
TKEVLDATLIHQSITGLYETRIDLSQLGGDGSPKKKRKVSS

In some embodiments, the S44P mutant NLS-dCas9-Clo051-NLS (“S44P Cas-CLOVER”, or “S44P CC”, or “S44P”, or “Cas-CLOVERv3”, or “CCv3”) fusion protein comprises an amino acid sequence that is at least 70%, at least 75%, at least 80%, at least 85%, at least 90%, at least 95%, at least 96%, at least 97%, at least 98% or at least 99% identical to the amino acid sequence set forth in SEQ ID NO: 56. In some embodiments, the fusion protein comprises an amino acid sequence that is at least 70%, at least 75%, at least 80%, at least 85%, at least 90%, at least 95%, at least 96%, at least 97%, at least 98% or at least 99% identical to the amino acid sequence set forth in SEQ ID NO: 58.

In some embodiments, the S44P mutant NLS-dCas9-Clo051-NLS (“S44P Cas-CLOVER”, or “S44P CC”, or “S44P”, or “Cas-CLOVERv3”, or “CCv3”) fusion protein comprises the amino acid sequence set forth in SEQ ID NO: 58 with one, two, three, four or five conservative amino acid substitutions.

The Cas-CLOVER v3 fusion protein can be encoded by a polynucleotide which comprises the nucleic acid sequence of SEQ ID NO: 59. The nucleic acid encoding the dCas9-Clo051 fusion protein can be DNA or RNA.

(SEQ ID NO: 59)
acauuugcuucugacacaacuguguucacuagcaaccucaaacagacaccaagcuugccaccauggcucccaagaag
aagcggaaggucGAGGGCAUCAAGAGCAACAUCAGCCUGCUGAAGGACGAGCUGAGAGGCCAGAUCAGCCACAUCUC
CCACGAGUACCUGAGCCUGAUCGACCUGGCCUUCGACcccAAGCAGAACCGGCUGUUCGAGAUGAAGGUGCUGGAAC
UGCUGGUCAACGAGUACGGCUUCAAGGGCAGACACCUCGGCGGCAGCAGAAAGCCUGAUGGCAUCGUGUACAGCACC
ACACUCGAGGACAACUUCGGCAUCAUCGUGGACACCAAGGCCUACAGCGAGGGCUACAGCCUGCCUAUCUCUCAGGC
CGACGAGAUGGAAAGAUACGUGCGCGAGAACAGCAACCGCGACGAGGAAGUGAACCCCAACAAGUGGUGGGAGAACU
UCAGCGAGGAAGUCAAAAAGUACUACUUCGUGUUCAUCAGCGGCAGCUUUAAGGGCAAGUUCGAGGAACAGCUGCGG
CGGCUGUCUAUGACCACAGGCGUUAACGGCAGCGCCGUGAACGUGGUCAAUCUGCUGCUGGGCGCCGAGAAGAUUAG
AAGCGGCGAGAUGACCAUCGAGGAACUGGAACGGGCCAUGUUCAACAACAGCGAGUUCAUCCUGAAGUACggcggag
gcggcagcgacaagaaguacucuaucggacuggccaucggcaccaacucuguuggaugggccgugaucaccgacgag
uacaaggugcccagcaagaaauucaaagugcugggcaacaccgaccggcacagcaucaagaagaaucugaucggcgc
ccugcuguucgacucuggcgaaacagccgaagccaccagacugaagagaaccgccagacggcgguacaccagaagaa
agaaccggaucugcuaccugcaagagaucuucagcaacgagauggccaagguggacgacagcuucuuccacagacug
gaagaguccuuccugguggaagaggacaagaagcacgagcggcaccccaucuucggaaauaucguggacgagguggc
cuaccacgagaaguaccccaccaucuaccaccugagaaagaaacugguggacagcaccgacaaggccgaccugcgac
ugaucuaucuggcccuggcucacaugaucaaguuccggggccacuuccugaucgagggcgaccugaauccugacaac
uccgacguggacaagcuguucauccagcuggugcagaccuacaaucagcuguucgaagagaaucccaucaacgccuc
uggcguggacgccaaagccauccugucugccagacugagcaagagcagacggcuggaaaaccugaucgcucagcugc
ccggcgagaagaagaauggccuguucggcaaccugauugcccugucucugggccugacaccuaacuucaaguccaac
uucgaucuggccgaggaugccaaacugcagcuguccaaggacaccuacgacgacgaccuggauaaccugcuggccca
gaucggcgaucaguacgccgacuuguuucuggccgccaagaaccugucugacgccauccugcugagcgacauccuga
gagugaacaccgagaucacaaaggccccucugagcgccucuaugaucaagagauacgacgagcaccaccaggaucug
acccugcugaaagcucucgucaggcagcagcugccagagaaguacaaagagauuuucuucgaccagagcaagaacgg
cuacgccggcuacauugauggcggagccagccaagaggaauucuacaaguucaucaagcccauccucgagaagaugg
acggcacagaggaacugcucgugaagcugaacagagaggaccugcugcggaagcagcggaccuucgacaauggcucu
aucccucaccagauccaccugggagagcugcacgccauucugcggagacaagaggacuuuuacccauuccugaagga
caaccgggaaaagauugagaagauccugaccuucaggauccccuacuacgugggaccacuggccagaggcaauagca
gauucgccuggaugaccagaaagagcgaggaaaccaucacacccuggaacuucgaagaggugguggacaagggcgcc
agcgcucaguccuucaucgagcggaugaccaauuucgacaagaaucugcccaacgagaaagugcugcccaagcacuc
ccugcuguacgaguacuucaccguguacaacgagcugaccaaagugaaauacgugaccgagggaaugagaaagcccg
ccuuucuguccggcgagcagaaaaaggccaucguggaucugcuguucaagaccaaccggaaagugaccgugaagcag
cugaaagaggacuacuucaagaaaaucgagugcuucgacuccguggaaaucagcggcguggaagaucgguucaaugc
cagccugggcacauaccacgaucugcugaaaauuaucaaggacaaggacuuccuggacaacgaggaaaacgaggaca
uccuugaggacaucgugcugacccugacacuguucgaggacagagagaugaucgaggaaaggcugaaaacauacgcc
caccuguucgacgacaaagucaugaagcaacugaagcggcggcgcuacacaggcuggggcagacugucuagaaagcu
gaucaacggcauccgggacaagcaguccggcaagaccauccuggacuuucugaaguccgacggcuucgccaacagaa
acuucaugcagcugauucacgacgacagccucaccuucaaagaggacauucagaaggcccagguuuccggccagggc
gauucucugcacgagcacauugccaaucuggccggcucucccgccauuaagaagggcauucugcagacagugaaagu
gguggaugagcuggucaaagugauggggagacacaagcccgagaacaucgugaucgaaauggccagagagaaccaga
ccacacagaagggccagaagaacucccgcgagagaaugaagcggaucgaagagggaaucaaagagcuggggagccag
auccugaaagaacaccccguggaaaacacccagcugcagaacgagaagcuguaccuguacuaccuccagaacggccg
ggauauguacguggaccaagagcuggacaucaaccgccugagcgacuacgauguggacgcuaucgugccccagucuu
uucugaaagaugacuccaucgacaacaaggugcugaccagaagcgauaagaaccggggcaagagcgacaacgugccc
ucugaagaggucgugaagaagaugaagaacuacuggcgacagcugcugaacgccaagcugauuacccagcggaaguu
cgauaaccugaccaaggccgagagaggcggccugucugaacuggauaaggccggcuucaucaagagacagcuggugg
aaacccggcagaucaccaaacacguggcacagauucuggacucccggaugaacaccaaauacgaugagaacgacaaa
cugauccgggaagugaaagucaucacccugaaguccaagcugguguccgauuuccggaaggauuuccaguucuacaa
agugcgggaaaucaacaacuaccaucacgcccacgacgccuaccugaaugccguuguuggaacagcccugaucaaga
aguaucccaagcuggaaagcgaguucguguacggcgacuacaagguguacgacgugcggaagaugaucgccaagagc
gagcaagagauuggaaaggcuaccgccaaauacuucuucuacuccaacaucaugaacuuuuucaagacagagaucac
ccucgccaacggcgagaucagaaagcggccucugaucgagacaaacggcgaaaccggcgagauugugugggauaagg
gcagagacuuugccacagugcggaaggugcucagcaugccccaagugaauaucgugaaaaagaccgaggugcagaca
ggcggcuucagcaaagaguccauucugccuaagcggaacuccgacaagcugaucgcccggaagaaggacugggaccc
caagaaauacggcggcuucgauagcccuaccguggccuauucugugcuggugguggccaaaguggaaaagggaaagu
ccaagaagcucaagagcgucaaagaacuccugggcaucaccaucauggaacgguccagcuucgagaagaacccuauc
gacuuucuggaagccaagggcuacaaagaagucaagaaggaccugaucaucaagcuccccaaguacagccuguucga
gcuggaaaauggccggaagcggaugcuggcuucugcuggcgaacugcagaagggaaacgaacuggcccugccuagca
aauaugugaacuuccuguaccuggccagccacuaugagaagcugaagggcagccccgaggacaaugagcagaagcag
cuuuucgucgagcagcacaagcacuaccuggacgagaucaucgagcagaucuccgaguucuccaagagagugauccu
ggccgacgccaaccuggacaagguucuguccgccuacaacaagcaccgggauaagcccaucagagagcaggccgaga
auaucauccaccuguuuacccugaccaaccugggagccccugccgccuucaaguacuucgacaccaccaucgaccgg
aagcgcuacaccagcaccaaagaagugcuggacgccacacugauccaccagagcaucaccggccuguacgagacacg
gaucgaucugucucagcuuggaggcgacggcagcccuaagaagaagagaaagguuuccagcuaauaaggcggccgcc
cucgccccggaccugcccucccgccaggugcacccaccugcaauaaaugcagcgaagccgggagaauucccucgccc
cggaccugcccucccgccaggugcacccaccugcaauaaaugcagcgaagccgggagcggccgcggauccccgggua
ccgaauucgauaucucuauagugucaccuaaauuuaauuaaaaaaaaaaaaaaaaaaaaaaaaaaaaaaaaaaaaaa
aaaaaaaaaaaaaaaaaaaaaaaaaaaaaaaaaaaaaaaaaA

gRNAs

As used herein, the term “guide sequence” or “spacer” in the context of a Cas-Clover system or a CRISPR-Cas9 system, comprises any polynucleotide molecule having sufficient complementarity with a target nucleic acid sequence to hybridize with the target nucleic acid sequence and direct sequence-specific binding of a nucleic acid-targeting complex to the target nucleic acid sequence. The guide sequence may comprise both RNA and DNA polynucleotides. The guide sequence may form a duplex with a target sequence. The duplex may be a DNA duplex, an RNA duplex, or a RNA/DNA duplex. The terms “guide molecule”, “guide RNA”, “gRNA”, “single guide RNA” and “sgRNA” are used interchangeably herein to refer to RNA-based molecules that are capable of forming a complex with a Cas-Clover or a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA may encompass RNA-based molecules having one or more chemically modifications (e.g., by chemical linking two ribonucleotides or by replacement of one or more ribonucleotides with one or more deoxyribonucleotides), as described herein. The guide sequence may also partially comprise RNA and DNA-based nucleotides in which the molecule is chimeric for RNA and DNA nucleobases (e.g., containing either ribose or deoxyribose sugars).

The Cas-Clover or the CRISPR/Cas9-based system may include two or more gRNAs, wherein the gRNAs target different DNA sequences. The target DNA sequences may be overlapping. The target sequence or protospacer is followed by a PAM sequence at the 3′ end of the protospacer. Different Type II CRISPR systems have differing PAM requirements. For example, the S. pyogenes Type II system uses an “NGG” sequence, where “N” can be any nucleotide.

The guide RNA or the guide RNA of a Cas-Clover protein or a CRISPR-Cas protein may comprise a tracr-mate sequence (encompassing a “direct repeat” in the context of an endogenous CRISPR system) and a guide sequence (also referred to as a “spacer” in the context of an endogenous CRISPR system). In some embodiments, the Cas-Clover or the CRISPR-Cas system or complex as described herein does not comprise and/or does not rely on the presence of a tracr sequence. In certain embodiments, the guide molecule may comprise, consist essentially of, or consist of a direct repeat sequence fused or linked to a guide sequence or spacer sequence.

In certain embodiments, the guide sequence or spacer length of the guide molecules is 15 to 50 nucleotides in length. In certain embodiments, the spacer length of the guide RNA is at least 15 nucleotides in length. In certain embodiments, the spacer length is from 15 to 17 nucleotides in length, from 17 to 20 nucleotides in length, from 20 to 24 nucleotides in length, from 23 to 25 nucleotides in length, from 24 to 27 nucleotides in length, from 27-30 nucleotides in length, from 30-35 nucleotides in length, or greater than 35 nucleotides in length.

In some embodiments, the guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 nucleotides in length.

As described above, the Cas-Clover system and the CRISPR/Cas9 system utilizes one or more targeting gRNAs that provides the targeting of the Cas-Clover system and the CRISPR/Cas9-based system. The gRNA may be a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA: tracrRNA duplex involved in the Type II Effector system. This duplex, which may include, for example, a 42-nucleotide crRNA and a 75-nucleotide tracrRNA, acts as a guide for the Cas9 to cleave the target nucleic acid.

### Expression Vectors and Host Cells

The disclosure also relates to vectors that include polynucleotides of the disclosure, host cells that are genetically engineered with the recombinant vectors, and the production of at least one protein scaffold by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. In some embodiments, the promoter is an EF-la promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors may include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotes (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors may include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124(8):1277-87).

Expression vectors may include at least one selectable drug resistance marker for isolation of cells modified by the compositions and methods of the disclosure. Selectable drug resistance markers of the disclosure may comprise wild-type or mutant Neo, DHFR, TYMS, FRANCE, RAD51C, GCS, MDR1, ALDH1, NKX2.2, or any combination thereof.

Those of ordinary skill in the art are knowledgeable in the numerous expression systems available for expression of a nucleic acid encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a preferred aspect, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenylation or transcription terminator sequences are typically incorporated into the vector. An example of a terminator sequence is the polyadenylation sequence from the bovine growth hormone gene. In some embodiments, the polyA sequence is an SV40 polyA sequence.

Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

The plasmid constructs described herein may be used to deliver nucleic acids encoding the transposase domains or fusion proteins described herein to a cell.

The transposase domains and fusion proteins described herein may also be delivered to a cell using mRNA constructs. Thus, in one embodiment, provided herein is an mRNA sequence encoding a transposase domain or a fusion protein described herein. Such mRNA sequences may be delivered to a cell using a nanoparticle, for example, a lipid nanoparticle. Examples of lipid nanoparticles are described in, e.g., International Patent Applications No. WO 2022/087148, No. WO 2022/182792, and No. WO 2023/141576, each of which is incorporated herein by reference in its entirety for examples of lipid nanoparticles that may be used to deliver mRNA constructs encoding the fusion proteins or transposase domains described herein.

Cells and Modified Cells

The transposase expressing polynucleotides described herein may be used in conjunction with a transposon to modify cells. The transposon can be a piggyBac™ (PB) transposon. In some embodiments, when the transposon is a PB transposon, the transposase is a piggyBac™ (PB) transposase a piggyBac-like (PBL) transposase or a Super piggyBac™ (SPB) transposase. Non-limiting examples of PB transposons are described in detail in U.S. Pat. Nos. 6,218,182; 6,962,810; 8,399,643 and PCT Publication No. WO 2010/099296, the content of each of which is incorporated herein by reference in its entirety for examples of transposases that may be delivered to cells using the polynucleotides described herein. The transposons can comprise a nucleic acid encoding a therapeutic protein or therapeutic agent. Examples of therapeutic proteins include those disclosed in PCT Publications No. WO 2019/173636 and No. WO 2020/051374, each of which is incorporated herein by reference in its entirety.

Thus, provided herein are modified cells comprising one or more transposon and one or transposase encoded by a transposase expressing polynucleotide or fusion proteins described herein. Cells and modified cells of the disclosure can be mammalian cells. Preferably, the cells and modified cells are human cells.

A cell modified using a transposase expressing polynucleotide described herein can be a germline cell or a somatic cell. Cells and modified cells of the disclosure can be immune cells, e.g., lymphoid progenitor cells, natural killer (NK) cells, T lymphocytes (T-cell), stem memory T cells (T_{SCM} cells), central memory T cells (T_{CM}), stem cell-like T cells, B lymphocytes (B-cells), antigen presenting cells (APCs), cytokine induced killer (CIK) cells, myeloid progenitor cells, neutrophils, basophils, eosinophils, monocytes, macrophages, platelets, erythrocytes, red blood cells (RBCs), megakaryocytes or osteoclasts. The modified cell can be differentiated, undifferentiated, or immortalized. The modified undifferentiated cell can be a stem cell. The modified undifferentiated cell can be an induced pluripotent stem cell. The modified cell can be a T cell, a hematopoietic stem cell, a natural killer cell, a macrophage, a dendritic cell, a monocyte, a megakaryocyte, or an osteoclast. The modified cell can be modified while the cell is quiescent, in an activated state, resting, in interphase, in prophase, in metaphase, in anaphase, or in telophase. The modified cell can be fresh, cryopreserved, bulk, sorted into sub-populations, from whole blood, from leukapheresis, or from an immortalized cell line. A detailed description for isolating cells from a leukapheresis product or blood is disclosed in in PCT Publication No. WO 2019/173636 and PCT/US2019/049816.

The methods of the disclosure can modify and/or produce a population of modified T cells, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the plurality of modified T cells in the population expresses one or more cell-surface marker(s) of a stem memory T cell (T_{SCM}) or a T_{SCM}-like cell; and wherein the one or more cell-surface marker(s) comprise CD45RA and CD62L. The cell-surface markers can comprise one or more of CD62L, CD45RA, CD28, CCR7, CD127, CD45RO, CD95, CD95 and IL-2RB. The cell-surface markers can comprise one or more of CD45RA, CD95, IL-2RB, CCR7, and CD62L.

The disclosure provides methods of expressing a CAR on the surface of a cell. The method comprises (a) obtaining a cell population; (b) contacting the cell population to a composition comprising a CAR or a sequence encoding the CAR, under conditions sufficient to transfer the CAR across a cell membrane of at least one cell in the cell population, thereby generating a modified cell population; (c) culturing the modified cell population under conditions suitable for integration of the sequence encoding the CAR; and (d) expanding and/or selecting at least one cell from the modified cell population that express the CAR on the cell surface. A more detailed description of methods for expressing a CAR on the surface of a cell is disclosed in PCT Publications No. WO 2019/049816 and No. WO 2020/051374, each of which is incorporated herein by reference in its entirety.

The present disclosure provides a cell or a population of cells wherein the cell comprises a composition comprising (a) an inducible transgene construct, comprising a sequence encoding an inducible promoter and a sequence encoding a transgene, and (b) a receptor construct, comprising a sequence encoding a constitutive promoter and a sequence encoding an exogenous receptor, such as a CAR, wherein, upon integration of the construct of (a) and the construct of (b) into a genomic sequence of a cell, the exogenous receptor is expressed, and wherein the exogenous receptor, upon binding a ligand or antigen, transduces an intracellular signal that targets directly or indirectly the inducible promoter regulating expression of the inducible transgene (a) to modify gene expression. In some embodiments, the car targets MUC1C.

The disclosure further provides a composition comprising the modified, expanded and selected cell population of the methods described herein.

The modified cells of disclosure (e.g., CAR T-cells) can be further modified to enhance their therapeutic potential. Alternatively, or in addition, the modified cells may be further modified to render them less sensitive to immunologic and/or metabolic checkpoints, for example by blocking and/or diluting specific checkpoint signals delivered to the cells (e.g., checkpoint inhibition) naturally, within the tumor immunosuppressive microenvironment.

The modified cells of disclosure (e.g., CAR T-cells) can be further modified to silence or reduce expression of (i) one or more gene(s) encoding receptor(s) of inhibitory checkpoint signals; (ii) one or more gene(s) encoding intracellular proteins involved in checkpoint signaling; (iii) one or more gene(s) encoding a transcription factor that hinders the efficacy of a therapy; (iv) one or more gene(s) encoding a cell death or cell apoptosis receptor; (v) one or more gene(s) encoding a metabolic sensing protein; (vi) one or more gene(s) encoding proteins that that confer sensitivity to a cancer therapy, including a monoclonal antibody; and/or (vii) one or more gene(s) encoding a growth advantage factor. Non-limiting examples of genes that may be modified to silence or reduce expression or to repress a function thereof include, but are not limited the illustrative inhibitory checkpoint signals, intracellular proteins, transcription factors, cell death or cell apoptosis receptors, metabolic sensing protein, proteins that that confer sensitivity to a cancer therapy and growth advantage factors that are disclosed in PCT Publication No. WO 2019/173636, which is incorporated herein by reference in its entirety.

The modified cells of disclosure (e.g., CAR T-cells) can be further modified to express a modified/chimeric checkpoint receptor. The modified/chimeric checkpoint receptor can comprise a null receptor, decoy receptor or dominant negative receptor. Illustrative null, decoy, or dominant negative intracellular receptors/proteins include, but are not limited to, signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of cytokines, cytokine receptors, chemokines and chemokine receptors are disclosed in PCT Publication No. WO 2019/173636, which is incorporated herein by reference in its entirety for examples of receptors and proteins that may be expressed in the cells disclosed herein.

In some aspects of the present disclosure, the cells of the present disclosure may be modified to decrease the expression of B2M. In some aspects of the present disclosure, the cells of the present disclosure may be modified to decrease the expression of CD3. The expression of B2M and/or CD3 may be decreased by using the gene editing compositions disclosed herein to target these genes. For example, cells may be modified using one of the Cas-Clover enzymes disclosed herein in conjunction with gRNAs targeting B2M and/or CD3.

Genome modification can comprise introducing a nucleic acid sequence, transgene and/or a genomic editing construct into a cell ex vivo, in vivo, in vitro or in situ to stably integrate a nucleic acid sequence, transiently integrate a nucleic acid sequence, produce site-specific integration of a nucleic acid sequence, or produce a biased integration of a nucleic acid sequence. The nucleic acid sequence can be a transgene.

The stable chromosomal integration can be a random integration, a site-specific integration, or a biased integration.

### Formulations, Dosages and Modes of Administration

The present disclosure provides formulations, dosages and methods for administration of the compositions and cells described herein. In one aspect, provided herein is a pharmaceutical composition comprising a polynucleotide for expressing a transposase described herein and a pharmaceutically acceptable carrier. In another aspect, provided herein is a pharmaceutical composition comprising a modified cell described herein and a pharmaceutically acceptable carrier.

The disclosed compositions and pharmaceutical compositions can comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means. In preferred embodiments, a composition comprising a modified cell described herein is administered intravenously, e.g., by intravenous infusion.

A composition of the disclosure can be prepared for use for parenteral (subcutaneous, intramuscular or intravenous) or any other administration particularly in the form of liquid solutions or suspensions. For parenteral administration, a composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection or infusion can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono-or di-or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

It can be desirable to deliver the disclosed polynucleotides to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono-or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In an aspect, the subject is a mammal. Preferably, the subject is human. The terms “subject” and “patient” are used interchangeably herein.

In some aspects, the treatment of a disease or disorder comprises adoptive cell therapy. For example, in an aspect, the disclosure provides modified cells that express a chimeric antigen receptor (CAR). The modified cells may be allogeneic or autologous to the patient. In some preferred embodiments, the modified cell is an allogeneic cell. In some embodiments, the modified cell is an autologous T-cell or a modified autologous CAR T-cell. In some preferred embodiments, the modified cell is an allogeneic T-cell or a modified allogeneic CAR T-cell.

In some embodiments, the disease or disorder treated in accordance with the methods described herein is a cancer. Non-limiting examples of cancer includes leukemia, acute leukemia, acute lymphoblastic leukemia (ALL), acute lymphocytic leukemia, B-cell, T-cell or FAB ALL, acute myeloid leukemia (AML), acute myelogenous leukemia, chronic myelocytic leukemia (CML), chronic lymphocytic leukemia (CLL), hairy cell leukemia, myelodyplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, hemangioma, metastatic disease, cancer related bone resorption, cancer related bone pain, and the like.

In another non-limiting example, the present disclosure provides methods of treating a metabolic liver disorder in a subject, the methods comprising administering to the subject: a) at least one therapeutically effective amount of at least one composition comprising a transposon of the present disclosure comprising a sequence encoding a therapeutic polypeptide; and b) at least one therapeutically effective amount of a composition comprising a nucleic acid sequence encoding at least one transposase. In some aspects, the metabolic liver disorder can be Ornithine Transcarbamylase (OTC) Deficiency and the at least one therapeutic protein can comprise ornithine transcarbamylase (OTC) polypeptide. In some aspects, the metabolic liver disorder can be methylmalonic acidemia (MMA) and the at least one therapeutic protein can comprise a methylmalonyl-CoA mutase (MUT1) polypeptide.

In a non-limiting example, the present disclosure provides methods of treating a hemophilia disease in a subject, the methods comprising administering to the subject: at least one therapeutically effective amount of at least one composition comprising a transposon of the present disclosure comprising a sequence encoding a therapeutic polypeptide; and b) at least one therapeutically effective amount of a composition comprising a nucleic acid sequence encoding at least one transposase. In some aspects, the hemophilia disease can be hemophilia A and the at least one therapeutic protein can comprise Factor VIII. In some aspects, the hemophilia disease can be hemophilia B and the at least one therapeutic protein can comprise Factor IX.

In a non-limiting example, the present disclosure provides methods of treating phenylketonuria (PKU) in a subject, the methods comprising administering to the subject: at least one therapeutically effective amount of at least one composition comprising a transposon of the present disclosure comprising a sequence encoding the phenylalanine hydroxylase gene; and b) at least one therapeutically effective amount of a composition comprising a nucleic acid sequence encoding at least one transposase.

In a non-limiting example, the present disclosure provides for methods of treating a disease or disorder in a subject by administering to the subject in need thereof a therapeutically effective amount of an LNP composition comprising a DNA transposon encoding a therapeutic protein and a transposase expressing polynucleotide mRNA encoding a piggyBac transposase as described herein. In some embodiments, the disease or disorder is cancer, a liver disease or disorder, a urea cycle disorder, a metabolic liver disorder or a hemophilia disease.

In a non-limiting example, the present disclosure provides for methods of treating a disease or disorder in a subject by administering to the subject in need thereof a therapeutically effective amount of a first LNP composition comprising a DNA transposon encoding a therapeutic protein, and a second LNP composition comprising a transposase expressing polynucleotide mRNA encoding a piggyBac transposase. In some embodiments, the disease or disorder is cancer, a liver disease or disorder, a urea cycle disorder, a metabolic liver disorder or a hemophilia disease.

In a non-limiting example, the present disclosure provides for methods of treating a disease or disorder in a subject by administering to the subject in need thereof a therapeutically effective amount of a first LNP composition comprising a DNA transposon encoding a therapeutic protein comprising a super minimal ITR, and a second LNP composition comprising an mRNA encoding a piggyBac transposase. In some embodiments, the disease or disorder is cancer, a liver disease or disorder, a urea cycle disorder, a metabolic liver disorder or a hemophilia disease. In some embodiments, the disease or disorder is an autoimmune disease. In one embodiment, the autoimmune disease is autoimmune neutropenia, Guillain-Barré syndrome, epilepsy, autoimmune encephalitis, Isaacs' syndrome, nevus syndrome, pemphigus vulgaris, deciduous pemphigus, bullous pemphigoid, acquired epidermolysis bullosa, gestational pemphigoid, mucous membrane pemphigoid, antiphospholipid syndrome, autoimmune anemia, myasthenia gravis, autoimmune Graves' disease, thyroid eye disease (TED), Goodpasture syndrome, multiple sclerosis, rheumatoid arthritis, lupus, idiopathic thrombocytopenia purpura (ITP), warm autoimmune hemolytic anemia (WAIHA), chronic inflammatory demyelinating polyneuropathy (CIDP), lupus nephritis, or membranous nephropathy.

The dosage of a pharmaceutical composition to be administered to a subject can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired.

In aspects where the compositions to be administered to a subject in need thereof are modified cells as disclosed herein, between about 1×10³ and about 1×10⁴ cells; between about 1×10⁴ and about 1×10⁵ cells; between about 1×10⁵ and about 1×10⁶ cells; between about 1×10⁶ and about 1×10⁷ cells; between about 1×10⁷ and about 1×10⁸ cells; between about 1×10⁸ and about 1×10⁹ cells; between about 1×10⁹ and about 1×10¹⁰ cells, between about 1×10¹⁰ and about 1×10¹¹ cells, between about 1×10¹¹ and about 1×10¹² cells, between about 1×10¹² and about 1×10¹³ cells, between about 1×10¹³ and about 1×10¹⁴ cells, between about 1×10¹⁴ and about 1×10¹⁵ cells, between about 1×10¹⁵ and about 1×10¹⁶ cells, between about 1×10¹⁶ and about 1×10¹⁷ cells, between about 1×10¹⁷ and about 1×10¹⁸ cells, between about 1×10¹⁸ and about 1×10¹⁹ cells; or between about 1×10¹⁹ and about 1×10²⁰ cells may be administered. In some embodiments, the cells are administered at a dose of between about 5×10⁶ and about 25×10⁶ cells.

In other embodiments, the dosage of cells may depend on the body weight of the person, e.g., between about 1×10³ and about 1×10⁴ cells; between about 1×10⁴ and about 1×10⁵ cells; between about 1×10⁵ and about 1×10⁶ cells; between about 1×10⁶ and about 1×10⁷ cells; between about 1×10⁷ and about 1×10⁸ cells; between about 1×10⁸ and about 1×10⁹ cells; between about 1×10⁹ and about 1×10¹⁰ cells, between about 1×10¹⁰ and about 1×10¹¹ cells, between about 1×10¹¹ and about 1×10¹² cells, between about 1×10¹² and about 1×10¹³ cells, between about 1×10¹³ and about 1×10¹⁴ cells, between about 1×10¹⁴ and about 1×10¹⁵ cells, between about 1×10¹⁵ and about 1×10¹⁶ cells, between about 1×10¹⁶ and about 1×10¹⁷ cells, between about 1×10¹⁷ and about 1×10¹⁸ cells, between about 1×10¹⁸ and about 1×10¹⁹ cells; or between about 1×10¹⁹ and about 1×10²⁰ cells may be administered per kg body weight of the subject.

A more detailed description of pharmaceutically acceptable excipients, formulations, dosages and methods of administration of the disclosed compositions and pharmaceutical compositions is disclosed in PCT Publication No. WO 2019/049816.

### Kits

In another aspect, provided herein is a kit comprising one or more composition comprising one transposase expressing polynucleotide described herein and a DNA transposon. In some embodiments, the one or more composition comprising a transposase expressing polynucleotide mRNA described herein and a DNA transposon is an LNP composition. In some embodiments, the one or more composition comprises a first LNP composition comprising the one transposase expressing polynucleotide mRNA described herein and a second LNP composition comprising a DNA transposon.

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof. In the context of nucleic acids, a promoter may be operatively linked to a nucleotide sequence encoding a transpose domain or fusion protein described herein, bringing the expression of the nucleotide sequence under the control of the promoter.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Nucleic acids of the disclosure may be single-or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo-and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Table 1, Table 2, and Table 3 below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Illustrative conservative substitutions are set out in Table 1.

TABLE 1
Conservative Substitutions I

| | |
|---|---|
| Side chain characteristics | Amino Acid |

| | | |
|---|---|---|
| Aliphatic | Non-polar | G A P I L V F |
| | Polar - uncharged | C S T M N Q |
| | Polar - charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table 2.

TABLE 2
Conservative Substitutions II

| | |
|---|---|
| Side Chain Characteristic | Amino Acid |

| | | |
|---|---|---|
| Non-polar (hydrophobic) | Aliphatic: | A L I V P |
| | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, illustrative conservative substitutions are set out in Table 3.

TABLE 3
Conservative Substitutions III

| Original Residue | Illustrative Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, identity between two sequences may be determined by using the stand-alone executable BLAST engine program for blasting two sequences (b12seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. In some embodiments, the sequence identify is determined over the entire length of a sequence. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

In certain embodiments, if a sequence has a certain sequence identity (e.g., 75%, 80%, 85%, 90%, 95%, 98%, or 99%) to a certain SEQ ID NO, the sequence and the sequence of the SEQ ID NO have the same length. In certain embodiments, if a sequence has a certain sequence identity (e.g., 75%, 80%, 85%, 90%, 95%, 98%, or 99%) to a certain SEQ ID NO, the sequence and the sequence of the SEQ ID NO only differ due to conservative amino acid substitutions.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

All documents cited herein, including any cross referenced or related patent or application is hereby incorporated herein by reference in its entirety for all purposes, unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### EXAMPLES

The Examples in this section are provided for illustration and are not intended to limit the invention.

### Example 1

### Effect of 3′UTR Composition on PiggyBac Transposase In Vitro Expression and Excision Activity in Hepatocytes

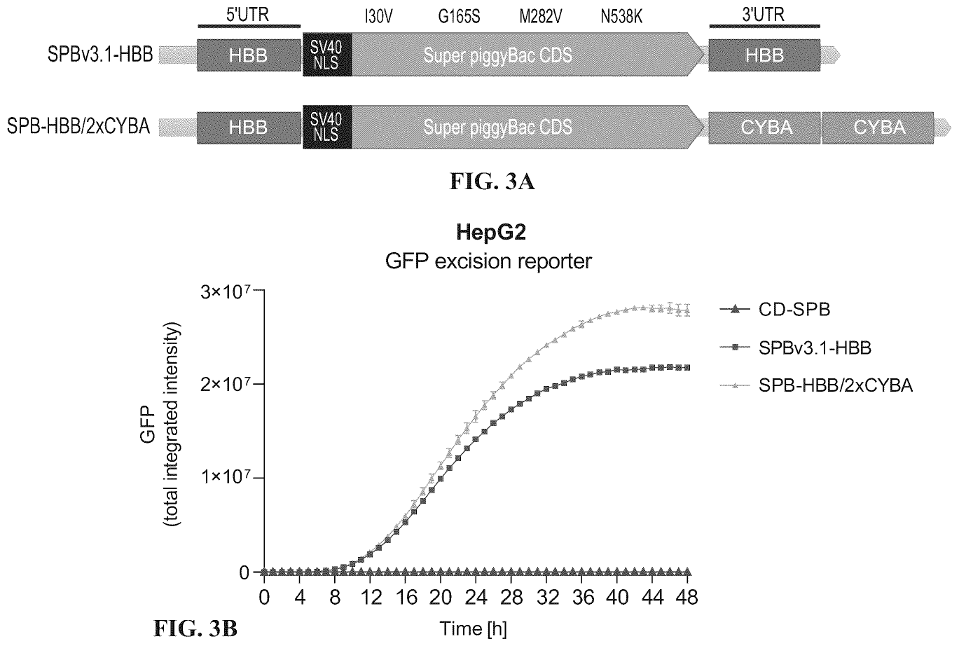

The effect of 3′-UTR composition on piggyBac transposase expression and excision activity was examined by constructing and comparing two piggyBac transposase encoding polynucleotides comprising different 3′-UTR sequences. The first piggyBac transposase encoding polynucleotide comprised in the 5′ to 3′ direction: a HBB 5′-UTR, a NLS coding sequence, a piggyBac transposase coding sequence comprising four hyperactive mutations at amino acids 130V; G165S; M282V and N538K, a HBB 3′-UTR and a poly A tail. The second piggyBac transposase encoding polynucleotide comprised in the 5′ to 3′ direction: a HBB 5′-UTR, a NLS coding sequence, a piggyBac transposase coding sequence comprising four hyperactive mutations at amino acids 130V; G165S; M282V and N538K, a 3′-UTR comprising a tandem CYBA 3′-UTR element joined by a linker sequence, and a polyA tail (FIG. 3A).

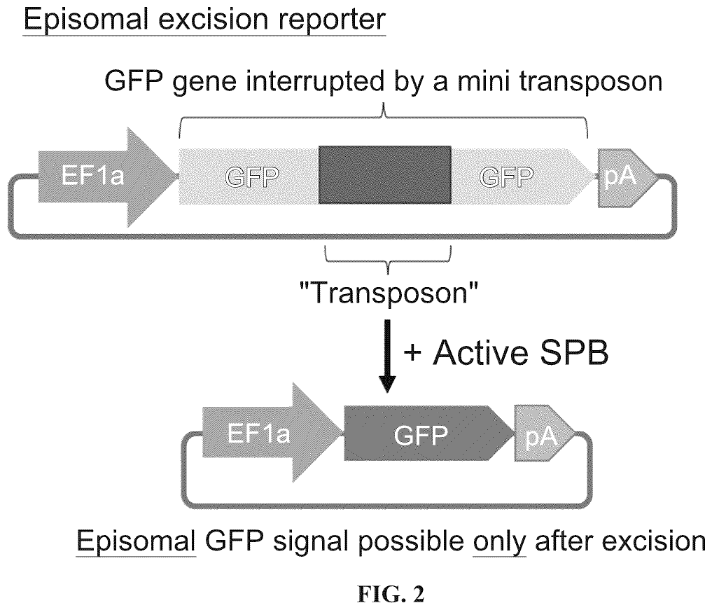

The excision activity of the two transposase encoding polynucleotides comprising a HBB 3′-UTR or tandem CYBA element 3′-UTR was determined using an episomal excision reporter plasmid (FIG. 2). Briefly, the episomal excision reporter plasmid comprises an EF-la promoter controlling expression of a GFP gene followed by a polyA sequence. The GFP coding sequence has been disrupted by the insertion of a mini-transposon, which prevents expression of functional GFP. If a transposase comprises excision activity, excision of the inserted mini-transposon by the transposase restores the full length GFP coding sequence resulting in intracellular expression of GFP. A catalytically dead piggyBac transposase lacking any appreciable transposase excision activity was used as a negative control.

On Day 0, 75,000 HepG2 cells were seeded into 48 well plates. On Day 1, 125 ng mRNA encoding for transposase (e.g., HBB 3′-UTR or 2X-CYBA 3′-UTR) and 375 ng transposon-interrupted GFP donor plasmid were delivered into each well of the 48-well plate and cells were co-transfected using Lipofectamine 3000 (Thermo Fisher) in accordance with the manufacturer's instructions. GFP expression was monitored over a 48-hour period time course by Incucyte. The results are shown in FIG. 3B.

As shown in FIG. 3B, piggyBac transposase encoding polynucleotides comprising the HBB 3′-UTR or 2X CYBA 3′-UTR were each capable of expressing the encoded piggyBac transposase mRNA in cells and excising the disrupting reporter mini-transposon. Excision of the mini-transposon restores a full-length GFP gene resulting in intracellular GFP expression. The transposase encoding polynucleotides comprising the 2X CYBA 3′-UTR demonstrated enhanced GFP expression in HepG2 hepatocyte cell line by approximately two-fold compared to transposase encoding polynucleotides comprising the HBB 3′-UTR. Cells transfected with polynucleotide encoding the catalytically dead piggyBac transposase negative control exhibited little to no GFP expression.

### Example 2

### Effect of 3′UTR Composition on PiggyBac Transposase In Vivo Expression and Excision Activity in Juvenile Mice

In a second experiment, the transposase encoding polynucleotides comprising the 2X CYBA 3′-UTR demonstrated enhanced serum Factor VIII (FVIII) levels in juvenile mice compared to transposase encoding polynucleotides comprising the HBB 3′-UTR.

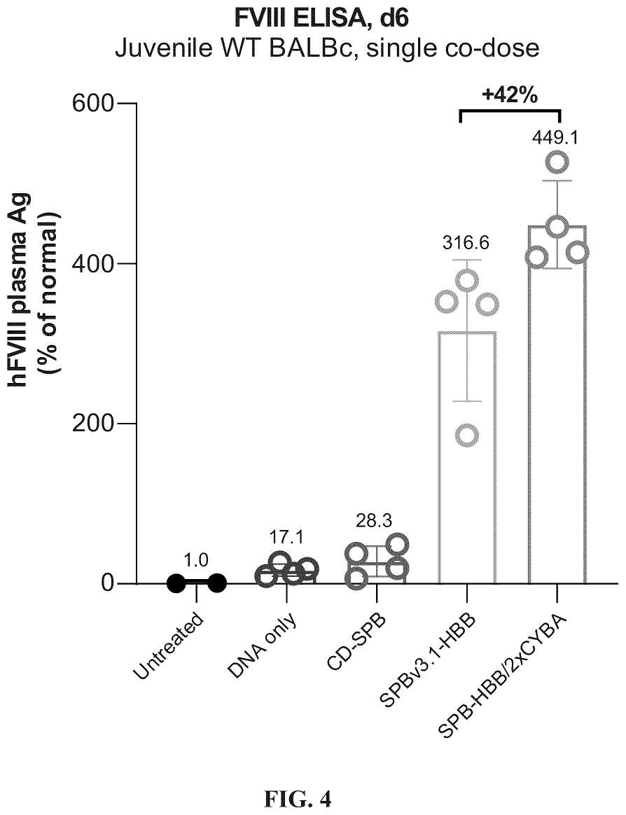

On Day 1, juvenile, wild type BALBc mice (N=4/group) were administered via intravenous injection a single 0.25 mg/kg dose of an LNP composition (C12-D8-35% C12-200; 41.84% DOPE; 20% CHOL; and 3.16% DM-PEG) encapsulating a DNA transposon comprising a nucleic acid encoding a modified Factor VIII gene under the control of a liver-specific promoter; or co-administered a single 0.25 mg/kg dose of the Factor VIII LNP composition and 0.35 mg/kg dose of an LNP composition (C12-D15 33.5% C12-200; 33.5% DOPE; 32% CHOL; and 1% DM-PEG) encapsulating an mRNA transposase encoding polynucleotide comprising a HBB 3′-UTR or a 2X CYBA 3′-UTR (or an mRNA transposase encoding polynucleotide comprising a nucleic acid encoding a catalytically dead transposase as a negative control). On Day 6, serum Factor VIII levels were determined from blood samples of treated and control animals using a Factor VIII ELISA. The results are shown in FIG. 4.

As shown in FIG. 4, the transposase encoding polynucleotides comprising the 2X CYBA 3′-UTR conferred a 42% increase in the serum Factor VIII levels in juvenile mice compared to the transposase encoding polynucleotides comprising the HBB 3′-UTR.

Example 3: Effect of 3′-UTR Composition on PiggyBac Transposase In Vivo Expression and Excision Activity in Adult Mice

In a related experiment, the transposase encoding polynucleotides comprising the 2X CYBA 3′-UTR demonstrated enhanced serum Factor VIII (FVIII) levels in adult mice compared to transposase encoding polynucleotides comprising the HBB 3′-UTR.

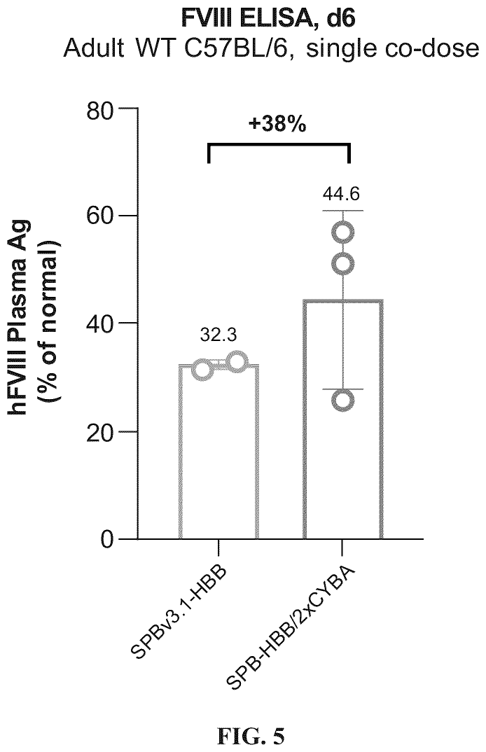

On Day 1, adult, wild type C57BL/6 mice (N=4/group) were administered via intravenous injection a single 0.25 mg/kg dose of an LNP composition (C12-D8-35% C12-200; 41.84% DOPE; 20% CHOL; and 3.16% DM-PEG) encapsulating a DNA transposon comprising a nucleic acid encoding a modified Factor VIII gene under the control of a liver-specific promoter; or co-administered a single 0.25 mg/kg dose of the Factor VIII LNP composition and a 0.5 mg/kg dose of an LNP composition (C12-D15-33.5% C12-200; 33.5% DOPE; 32% CHOL; and 1% DM-PEG) encapsulating an mRNA transposase encoding polynucleotide comprising a HBB 3′-UTR or a 2X CYBA 3′-UTR. On Day 6, serum Factor VIII levels were determined from blood samples drawn from treated and control animals using a Factor VIII ELISA. The results are shown in FIG. 5.

As shown in FIG. 5, the transposase encoding polynucleotide comprising the 2X CYBA 3′-UTR conferred a 38% increase in the serum Factor VIII levels in adult mice compared to the transposase encoding polynucleotide comprising the HBB 3′-UTR.

### Example 4

### Effect of the Addition of miR-142-3p Binding Sites to the 3′-UTR on PiggyBac Transposase Polynucleotides on In Vitro Expression and Excision Activity in Hepatocytes

The effect of the addition of miR-142-3p binding sites to the 3′-UTR on piggyBac transposase mRNA on in vitro expression and excision activity in hepatocytes was examined using HepG2 cells.

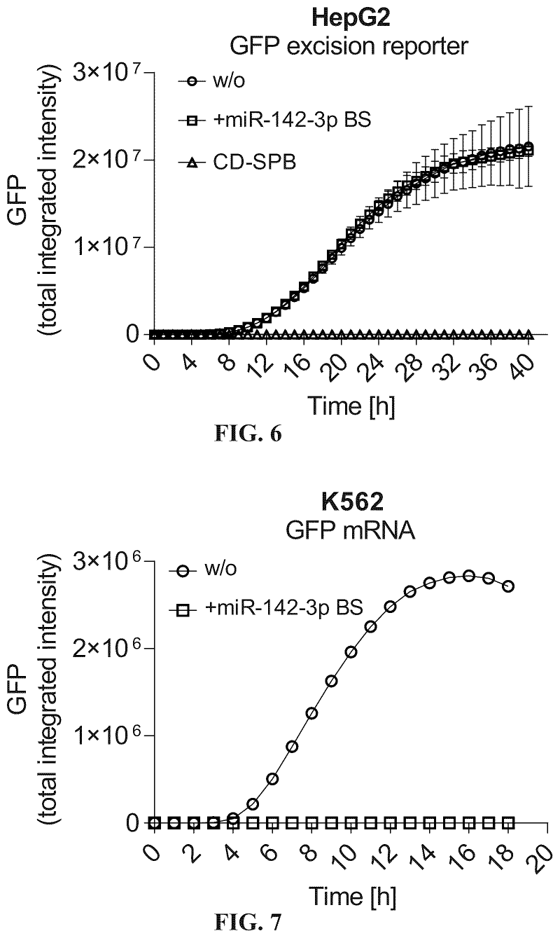

Briefly, on Day 0, 75,000 HepG2 cells were seeded into 48 well plates. On Day 1, 125 ng mRNA encoding for transposase comprising or lacking four miR-142-3p binding sites in the 3′-UTR and 375 ng transposon-interrupted GFP donor plasmid described in Example 1 were delivered into each well of the 48-well plate and cells were co-transfected using Lipofectamine 3000 reagent (Thermo Fisher) in accordance with the manufacturer's instructions. GFP expression was monitored over a 40-hour period by Incucyte. The results are shown in FIG. 6.

As shown in FIG. 6, the addition of four miR-142-3p binding sites separated by linker sequences to the 3′-UTR on piggyBac transposase had no observable effect on the encoded transposase excision activity over a 40 hr period compared to the 3′-UTR lacking the four miR-142-3p binding sites.

### Example 5

### Addition of miR-142-3p Binding Sites to the 3′-UTR of an mRNA encoding GFP Decreases GFP Expression in Hematopoietic Cells

The effect of the addition of miR-142-3p binding sites to the 3′-UTR of an mRNA encoding GFP was examined by constructing and comparing two DNA plasmids comprising a GFP gene encoding mRNAs comprising or lacking four miR-142-3p binding sites within the 3′-UTR. The first GFP encoding mRNA comprises the miR-142-3p binding sites and the second GFP encoding mRNA lacking the miR-142-3p binding sites.

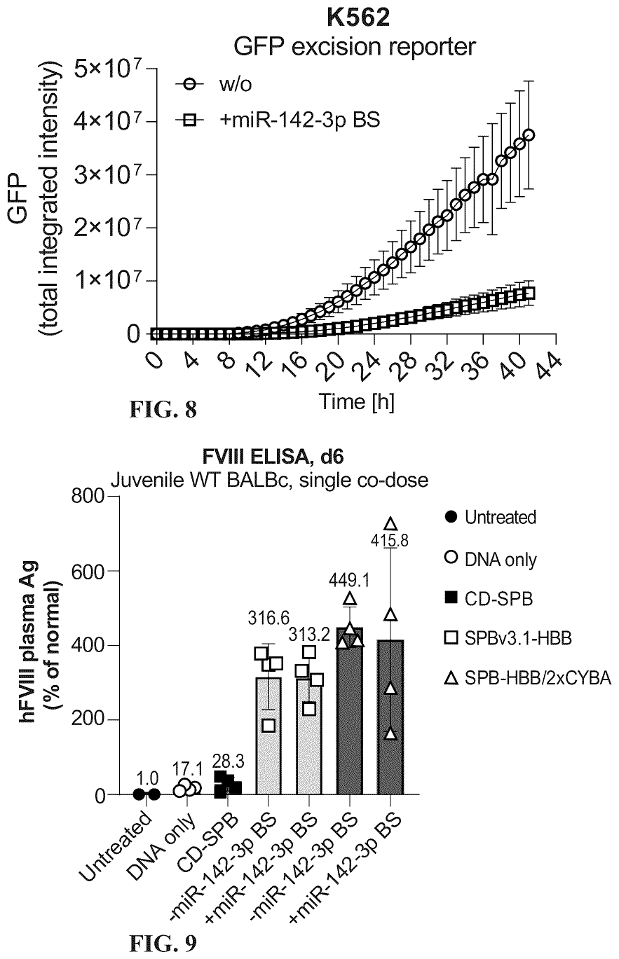

On Day 0, about 250,000 K562 cells were nucleofected using 20 μl of SF buffer and program FF-120. Each reaction contained 830 ng of GFP encoding mRNAs comprising or lacking four miR-142-3p binding sites within the 3′-UTR. GFP expression from K562 cells transfected with each mRNA was monitored over an 18-hour period by Incucyte. The results are shown in FIG. 7.

As shown in FIG. 7, GFP expression in K562 cells nucleofected with the mRNA encoding the GFP lacking the four miR-142-3p binding sites is detected at about four hours post-nucleofection and increases to a maximum expression level at about 16 hours post transfection. In contrast, GFP expression in K562 cells transfected with the mRNA encoding the GFP comprising the four miR-142-3p binding sites remains essentially undetectable throughout the 18-hour post-nucleofection monitoring period resulting in a 99% inhibition of GFP expression in hematopoietic cells.

### Example 6

### Addition of miR-142-3p Binding Sites to the 3′-UTR on PiggyBac Transposase Polynucleotides Decreases PiggyBac Transposase Expression and Excision Activity in Hematopoietic Cells

The effect of the addition of miR-142-3p binding sites to the 3′-UTR on piggyBac transposase mRNA on in vitro expression and excision activity in hematopoietic cells was analyzed.

Two mRNAs encoding a piggyBac transposase comprising or lacking four miR-142-3p binding sites with the 3-'UTR were constructed, and the transposon excision activity of the encoded transposases was determined in K562 hematopoietic cells using the episomal excision reporter plasmid system described in FIG. 2.

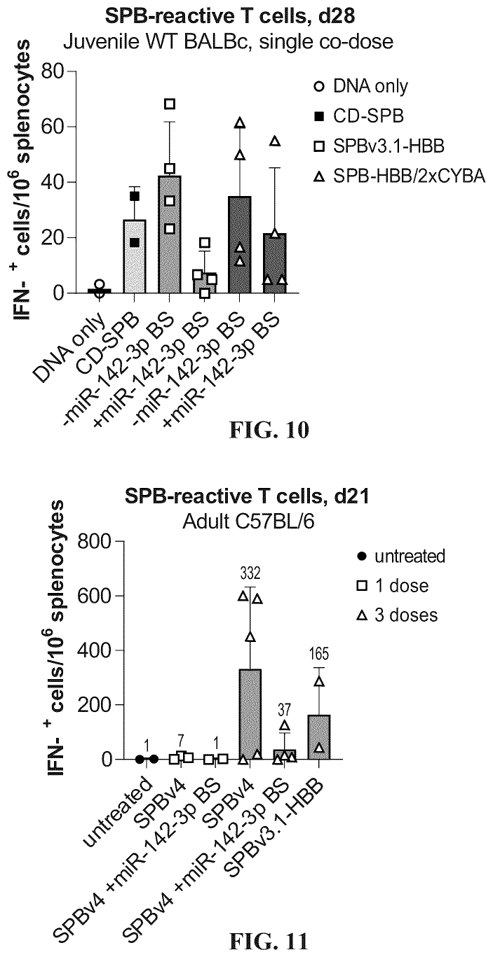

On Day 0, about 250,000 K562 cells were nucleofected using 20 μl of SF buffer and program FF-120. Each reaction contained 208 ng of piggyBac transposase encoding mRNAs comprising or lacking four miR-142-3p binding sites within the 3′-UTR, and 400 ng transposon-interrupted GFP donor plasmid described in Example 1. GFP expression from nucleofected K562 was monitored over a 42-hour period by Incucyte. The results are shown in FIG. 8.

As shown in FIG. 8, transposase-mediated GFP reporter expression in K562 cells transfected with the mRNA encoding the piggyBac transposase lacking the four miR-142-3p binding sites is detected at about twelve hours post-transfection and linearly increases to a maximum expression level at the end of the 42 hours post transfection time course. In contrast, transposase-mediated GFP reporter expression in K562 cells nucleofected with the mRNA encoding the piggyBac transposase comprising the four miR-142-3p binding sites remains essentially undetectable until about 18 hours post transfection monitoring and exhibited a 79% inhibition of piggyBac transposase excision activity in hematopoietic cells at the end of the 42-hour monitoring period.

### Example 7

### Effect of 3′-UTR Composition on PiggyBac Transposase In Vivo Expression, Excision Activity and T-cell Reactivity in Juvenile Mice

The effect of the addition of miR-142-3p binding sites to the 3′-UTR of an mRNA encoding piggyBac transposase was examined in vivo by constructing and comparing two mRNAs encoding a piggyBac transposase with a HBB 3′-UTR or 2X CYBA 3′-UTR (illustrated in FIG. 3A) comprising or lacking four miR-142-3p binding sites in the 3′-UTR.

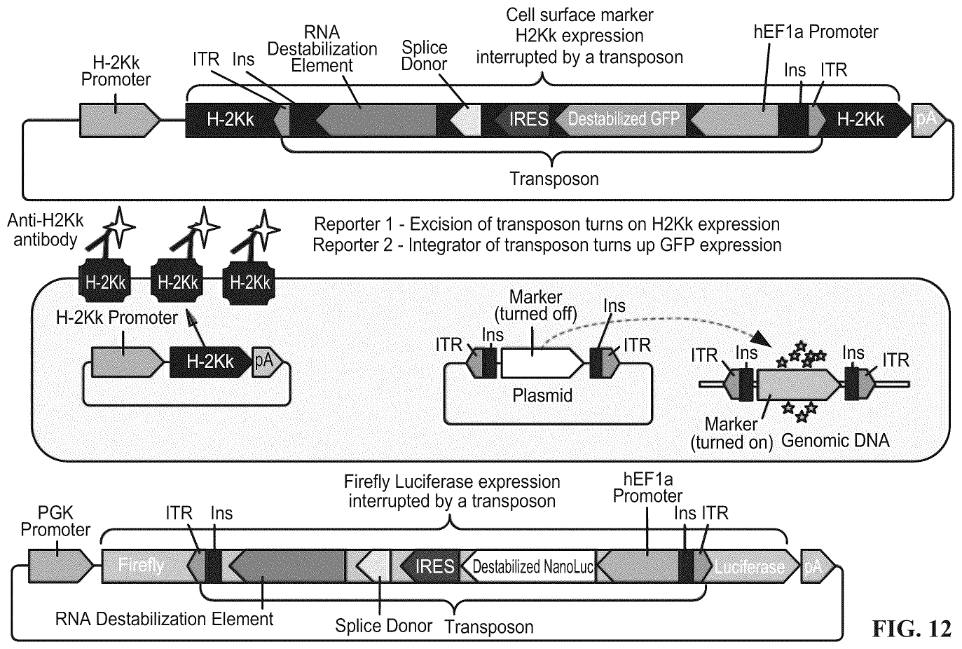

On Day 1, juvenile, wild type BALBc mice (N=4/group) were administered via intravenous injection a single 0.25 mg/kg dose of an LNP composition (C12-D8-35% C12-200; 41.84% DOPE; 20% CHOL; and 3.16% DM-PEG) encapsulating a DNA transposon comprising a nucleic acid encoding a modified Factor VIII gene under the control of a liver-specific promoter; or co-administered a single 0.25 mg/kg dose of the Factor VIII LNP composition and 0.35 mg/kg dose of an LNP composition (C12-D15-33.5% C12-200; 33.5% DOPE; 32% CHOL; and 1% DM-PEG) encapsulating an mRNA piggyBac transposase encoding polynucleotide comprising a HBB 3′-UTR comprising or lacking four miR-142-3p binding sites or an mRNA piggyBac transposase encoding polynucleotide comprising a 2X CYBA 3′-UTR comprising or lacking four miR-142-3p binding sites (or an mRNA transposase encoding polynucleotide comprising a nucleic acid encoding a catalytically dead transposase as a negative control). On Day 6, serum Factor VIII levels were determined from blood samples of untreated, treated and control animals using a Factor VIII ELISA. The results are shown in FIG. 9. As shown in FIG. 9, the addition of the four miR-142-3p binding sites to the mRNA transposase encoding polynucleotides comprising a HBB 3-'UTR or a 2X CYBA 3′-UTR exhibited no effect on the serum Factor VIII levels in juvenile mice compared to the mRNA transposase encoding polynucleotides lacking the four miR-142-3p binding sites.

In a second experiment, the effect of the addition of miR-142-3p binding sites to the 3′-UTR of an mRNA encoding piggyBac transposase on the number of piggyBac transposase reactive T-cells was examined in vivo by constructing and comparing mRNAs encoding a piggyBac transposase with a HBB 3′-UTR or 2X CYBA 3′-UTR (illustrated in FIG. 3A) comprising or lacking four miR-142-3p binding sites in the 3′-UTR.

On Day 1, juvenile, wild type BALBc mice (N=4/group) were administered via intravenous injection a single 0.25 mg/kg dose of an LNP composition (C12-D8-35% C12-200; 41.84% DOPE; 20% CHOL; and 3.16% DM-PEG) encapsulating a DNA transposon comprising a nucleic acid encoding a modified Factor VIII gene under the control of a liver-specific promoter; or co-administered a single 0.25 mg/kg dose of the Factor VIII LNP composition and 0.35 mg/kg dose of an LNP composition (C12-D15-33.5% C12-200; 33.5% DOPE; 32% CHOL; and 1% DM-PEG) encapsulating an mRNA piggyBac transposase encoding polynucleotide comprising a HBB 3′-UTR comprising or lacking four miR-142-3p binding sites or an mRNA piggyBac transposase encoding polynucleotide comprising a 2X CYBA 3′-UTR comprising or lacking four miR-142-3p binding sites (or an mRNA transposase encoding polynucleotide comprising a nucleic acid encoding a catalytically dead transposase as a negative control).

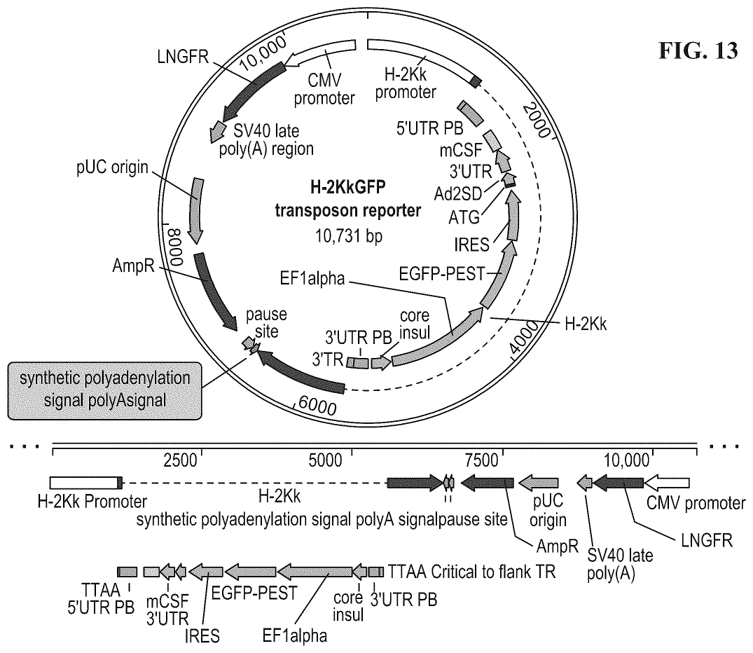

On Day 28, treated and control mice were euthanized, their spleens extracted, splenocytes isolated and the number of piggyBac transposase reactive T-cells (IFN gamma positive T-cells/10e6 splenocytes) was calculated using an ELISpot assay from treated and control animals. The results are shown in FIG. 10.

As shown in FIG. 10, the addition of the four miR-142-3p binding sites to the mRNA transposase encoding polynucleotides comprising a HBB 3-'UTR or a 2X CYBA 3′-UTR resulted in a decrease in the number of IFN gamma positive T-cells in juvenile mice compared to the corresponding mRNA transposase encoding polynucleotides lacking the four miR-142-3p binding sites.

### Example 8

### Effect of 3′UTR Composition on T-cell Reactivity After Repeat Dosing of PiggyBac Transposase Polynucleotides in Adult Mice

The effect of the addition of miR-142-3p binding sites to the 3′-UTR of an mRNA encoding piggyBac transposase on the number of piggyBac transposase reactive T-cells after single or repeat dosing was examined in vivo by constructing and comparing mRNAs encoding a piggyBac transposase with a 2X CYBA 3′-UTR (illustrated in FIG. 3A) comprising or lacking four miR-142-3p binding sites in the 3′-UTR.

On Day 1, adult, wild type C57BL/6 mice (N=5/group) were co-administered via intravenous injection a single 0.25 mg/kg dose of an LNP composition (C12-D8-35% C12-200; 41.84% DOPE; 20% CHOL; and 3.16% DM-PEG) encapsulating a DNA transposon comprising a nucleic acid encoding a modified Factor VIII gene under the control of a liver-specific promoter and 0.5 mg/kg dose of an LNP composition (C12-D15-33.5% C12-200; 33.5% DOPE; 32% CHOL; and 1% DM-PEG) encapsulating: an mRNA piggyBac transposase encoding polynucleotide comprising a HBB 3′-UTR; an mRNA piggyBac transposase encoding polynucleotide comprising a tandem 2X CYBA 3′-UTR or an mRNA piggyBac transposase encoding polynucleotide comprising a 2X CYBA 3′-UTR and comprising four miR-142-3p binding sites.

In addition, on Day 1, one group of mice were co-administered a 0.25 mg/kg dose of an LNP composition encapsulating a DNA transposon comprising a nucleic acid encoding a modified Factor VIII gene gene under the control of a liver-specific promoter and 0.5 mg/kg dose of an LNP composition encapsulating: an mRNA piggyBac transposase encoding polynucleotide comprising a HBB 3′-UTR; an mRNA piggyBac transposase encoding polynucleotide comprising a tandem 2X CYBA 3′-UTR or an mRNA piggyBac transposase encoding polynucleotide comprising a 2X CYBA 3′-UTR and comprising four miR-142-3p binding sites. On Day 7, treated mice were re-dosed by co-administration of a 0.15 mg/kg dose of an LNP composition encapsulating a DNA transposon and 0.5 mg/kg dose of the LNP composition encapsulating the same mRNA piggyBac transposase encoding polynucleotide and on Day 14, treated mice were administered a third 0.5 mg/kg dose of the LNP composition encapsulating the same mRNA piggyBac transposase encoding polynucleotide.

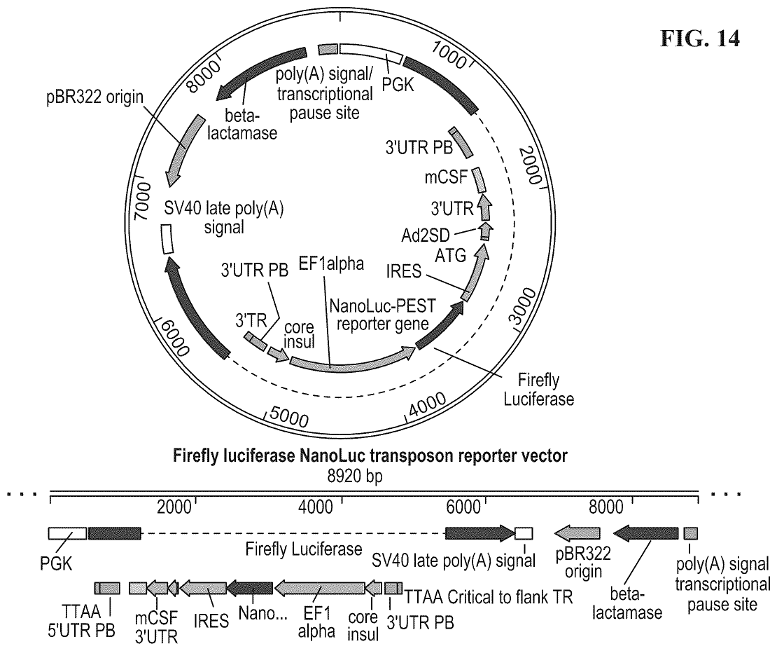

On Day 21, treated and control mice were euthanized, their spleens extracted, splenocytes isolated and the number of piggyBac transposase reactive T-cells (IFN gamma positive T-cells/10e6 splenocytes) was calculated using an ELISpot assay from treated and control animals. The results are shown in FIG. 11.

As shown in FIG. 11, the addition of the four miR-142-3p binding sites to the mRNA transposase encoding polynucleotides resulted in a substantial decrease in the number of IFN gamma positive T-cells after repeated dosing in adult mice compared to repeat dosing of the mRNA transposase encoding polynucleotides lacking the four miR-142-3p binding sites.

### Example 9

### Effect of the Number of Hyperactive Mutations and ITR Composition on PiggyBac Transposase Polynucleotides Transposon Integration and Excision Activity

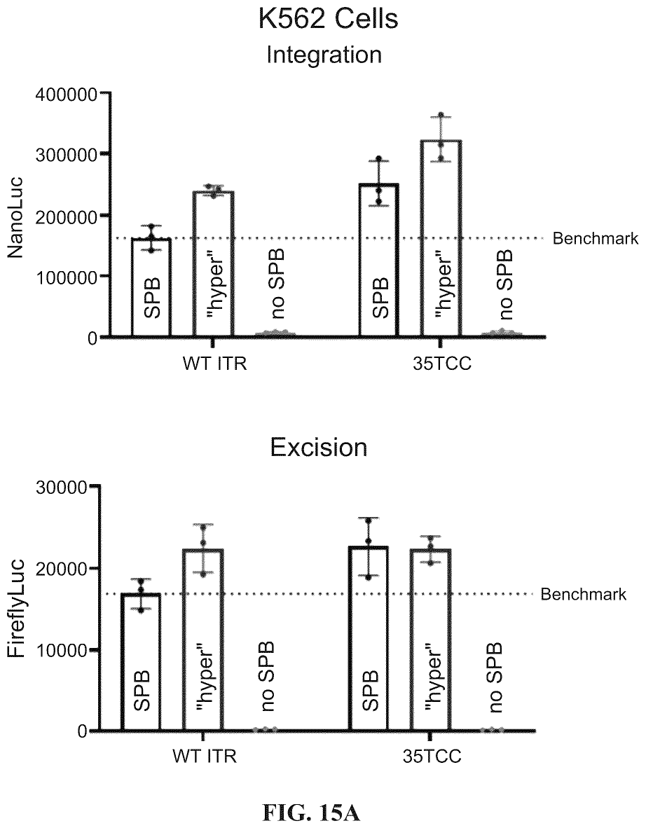

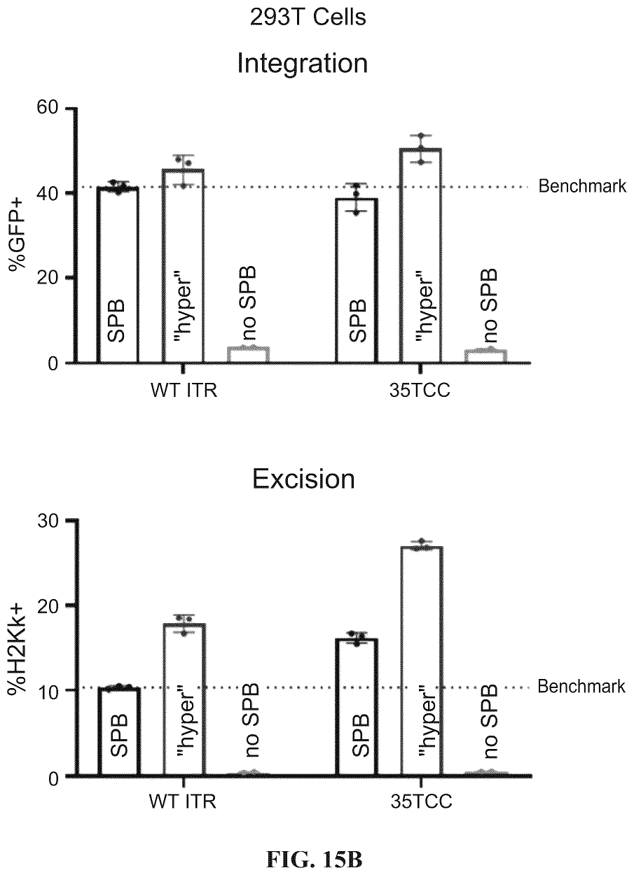

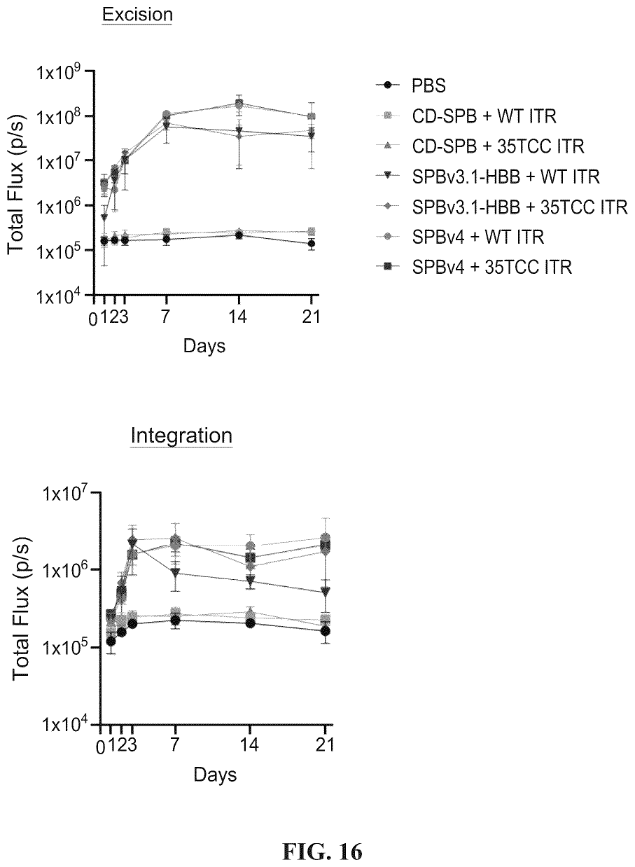

The integration and excision activities of piggyBac transposase encoding polynucleotides encoding a piggyBac transposase comprising four hyperactive mutations (130V; G165S; M282V and N538K) or five hyperactive mutations (130V; G165S; M226F, M282V and N538K) was determined using transposons comprising either wild type left end (LE) and right end (RE) piggyBac ITRs or a wild type RE piggyBac ITR and a LE piggyBac ITR comprising a 35TCC mutation. The reporter systems used to test for integration or excision are shown in FIGS. 12-14. FIG. 12 shows a schematic of the assays and FIGS. 13 and 14 show the vector map of the plasmids used.

In one instance, the piggyBac transposase encoding polynucleotides encoding a piggyBac transposase comprising four or five hyperactive mutations were nucleofected into K562 cells and the cells were co-transfected with a dual excision/integration luciferase reporter vector (FIG. 14) comprising wild type piggyBac LE and RE ITRs or a wild type RE piggyBac ITR and a LE piggyBac ITR comprising a 35TCC mutation. The vector was designed such that a firefly luciferase open reading frame is disrupted by a SPB transposon. Initially, firefly luciferase is not expressed, but SPB-mediated excision of the transposon and seamless repair results in expression. The transposon itself expresses a destabilized Nanoluc luciferase mRNA. Nanoluc expression from the episomal vector is unstable as the mRNA lacks a poly A tail and contains 3′ destabilization element. Integration of the transposon into genomic DNA allows the mRNA to pick up a polyA and splice out the destabilization element using a splice donor sequence on the transposon, leading to luciferase expression.

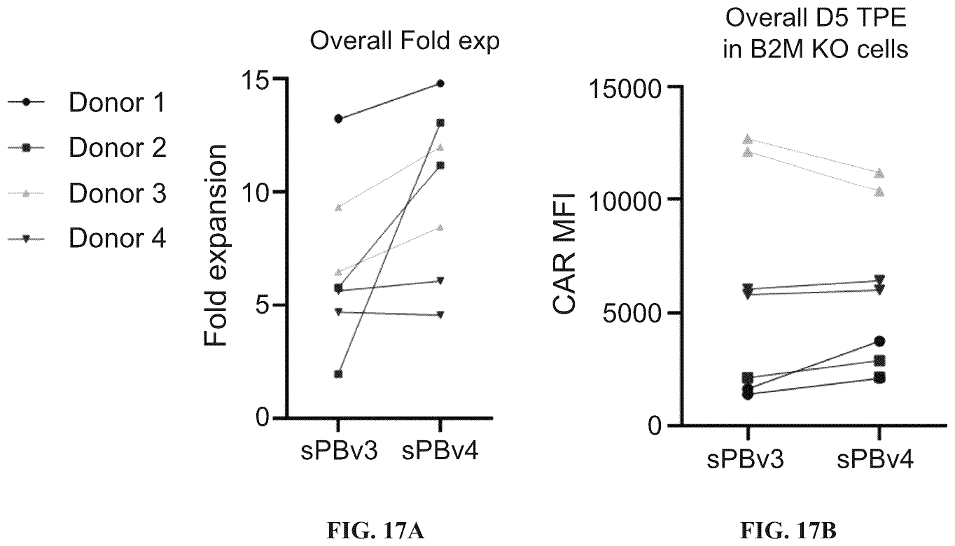

K562 cells were nucleofected using 20 μl of SF buffer and program FF-120. Each reaction contained 50 ng of the dual luciferase reporter and 500 ng of a transposase-expressing plasmid. One day post transfection, luciferase signal was measured using Promega's dual luciferase reagents and a plate reader. Results are shown in FIG. 15A.

As shown in FIG. 15A, the addition of the 5^{th} hyperactive M226F mutation to the piggyBac transposase sequence resulted in an approximately 50% enhancement in integration activity for transposons comprising the wild type ITRs, whereas both hyperactive mutant piggyBac transposases exhibit enhanced integration activity for transposons comprising the 35TCC LE ITR. The addition of the 5^{th} hyperactive M226F mutation and the 35TCC LE ITR resulted in a two-fold enhancement of integration activity. The addition of the 5^{th} hyperactive mutation to the piggyBac transposase sequence enhanced excision activity for transposons comprising either wild type ITRs or the 35TCC LE ITR compared to only 4 hyperactive mutations and both hyperactive transposases exhibited enhanced excision activity for transposons comprising the 35TCC LE ITR, though no additional enhancement in excision activity was observed for the transposase comprising the M226F mutation.

In another instance, the piggyBac transposase encoding polynucleotides encoding a piggyBac transposase comprising four or five hyperactive mutations were transfected into 293T cells and the cells were co-transfected with a dual excision/integration H2Kk/GFP reporter vector (FIG. 13) comprising wild type piggyBac LE and RE ITRs or a wild type RE piggyBac ITR and a LE piggyBac ITR comprising a 35TCC mutation. The vector was designed such that a H2Kk open reading frame is disrupted by a SPB transposon. Initially, H2Kk is not expressed, but SPB-mediated excision of the transposon and seamless repair results in expression. H2Kk is a cell-surface protein, and its expression may be detected on the cell surface using a fluorescent anti-H2Kk antibody. The transposon itself expresses a destabilized GFP mRNA. GFP expression from the episomal vector is unstable as the mRNA lacks a polyA tail and contains 3′ destabilization element. Integration of the transposon into genomic DNA allows the mRNA to pick up a polyA and splice out the destabilization element using a splice donor sequence on the transposon, leading to GFP expression.

On Day 0, 120,000 HEK293T cells were seeded into 24 well plates. On Day 1, 10 ng plasmid encoding for transposase (e.g., a piggyBac transposase comprising 4 or 5 hyperactive mutants), and 490 ng of the dual H2Kk/GFP reporter plasmid were delivered into specified wells of the 24-well plate and cells were transfected using JetPrime reagent (Polyplus) in accordance with the manufacturer's instructions. On Day 2, transfected cells were passaged and the remaining cells were stained for H2Kk expression and analyzed by flow cytometry to determine the percentage of H2Kk positive cells. The cells were passaged again on Day 5. On Day 8 the cells were analyzed by flow cytometry to determine the percentage of GFP positive cells. The results are shown in FIG. 15B.

As shown in FIG. 15B, the addition of the 5^{th} M226F hyperactive mutation exhibited little to no benefit for integration activity compared to the hyperactive transposase comprising only 4 hyperactive mutations; however, the addition of the 5^{th} hyperactive M226F mutation to the piggyBac transposase sequence resulted in an approximately 50% enhancement in excision activity for transposons comprising the wild type ITRs, whereas both hyperactive mutant piggyBac transposases exhibit enhanced excision activity for transposons comprising the 35TCC LE ITR. The addition of the 5^{th} hyperactive M226F mutation and the 35TCC LE ITR resulted in a 2.5-fold enhancement of excision activity.

### Example 10

### Improved PiggyBac Transposase Polynucleotides and 35TCC LE ITR Enhance Transposase Integration and Excision Activity in Juvenile Mice

A dual excision/integration luciferase reporter system was used to test wild type ITRs and 35TCC LE ITR effect on transposase activity of piggyBac transposase comprising 5 hyperactive mutations and a 3′-UTR comprising a tandem 2X CYBA 3′-UTR sequence and four miR-142-3p binding sites or 4 hyperactive mutations and a HBB 3′-UTR of in vivo integrating or excising engineered transposons. The reporter system comprises a firefly luciferase open reading frame disrupted by a piggyBac transposon. Initially, firefly luciferase is not expressed, but tranposase-mediated excision of the transposon and seamless repair results in expression. The transposon itself expresses a destabilized nanoluc luciferase mRNA. NanoLuc expression from the episomal vector is unstable, since the mRNA lacks a polyA tail and contains 3′ destabilization element. Integration of the transposon into genomic DNA allows the mRNA to utilize a genomic polyA sequence and splice out the destabilization element using a splice donor sequence on the transposon, leading to luciferase expression.

On Day 1, juvenile, wild type BALBc mice (N=3/group) were co-administered via intravenous injection a single 0.25 mg/kg dose of an LNP composition (C12-D8-35% C12-200; 41.84% DOPE; 20% CHOL; and 3.16% DM-PEG) encapsulating the DNA dual integration/excision reporter comprising a transposon containing wild type LE and RE ITRs or a transposon containing a 35TCC LE ITR and a wild type RE ITR and 0.5 mg/kg dose of an LNP composition (C12-D15-33.5% C12-200; 33.5% DOPE; 32% CHOL; and 1% DM-PEG) encapsulating: an mRNA piggyBac transposase encoding polynucleotide comprising 4hyperactive mutations and a HBB 3′-UTR or an mRNA piggyBac transposase encoding polynucleotide comprising 5 hyperactive mutations and a 3′-UTR comprising a tandem 2X CYBA 3′-UTR and four miR-142-3p binding sites. As a negative control, LNP compositions were administered as above comprising an mRNA encoding a catalytically dead version of the piggyBac transposase.

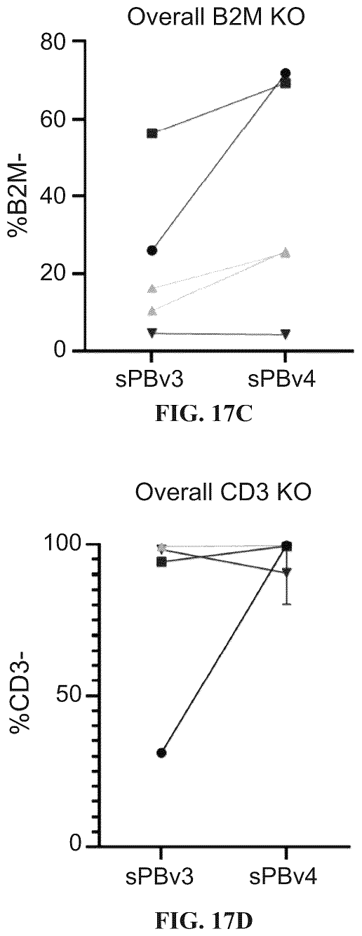

Luciferase activity was measured on Days 1, 2, 3 7, 14 & 21 for treated and control animals and the observed total flux for each day and construct as shown in FIG. 16. As shown in FIG. 16, each piggyBac transposase polynucleotide was capable of expressing transposase and capable in vivo of integrating and excising transposon comprising wild type ITRs or 35TCC LE ITR in juvenile mice, though a slight enhancement of in vivo integration activity was observed for the piggyBac transposase comprising 5 hyperactive mutations and a 3′-UTR comprising tandem 2X CYBA 3′-UTR elements and four miR-142-3p binding sites.

### Example 11

### Illustrative Method for the Preparation of 5′CleanCap PiggyBac Transposase mRNA for Use in LNP Compositions

This Example provides an illustrative method for preparing 5′CleanCap mRNA encoding a SPB transposase comprising a 5′hemagglutinin tag. The method may be used to preparing 5′CleanCap mRNA encoding additional piggyBac transposases

The DNA plasmid pRT-HA-SPB-CC-AG encodes Super piggyBac transposase comprising a 5′-hemagglutinin tag corresponding to amino acids 98-106 (“HA-SPB”). This plasmid was used as a template for in vitro transcription reactions to produce mRNA encoding HA-SPB further comprising a 5′-CAP.

Briefly, approximately 10 ug of supercoiled pRT-HA-SPB-CC-AG was added to a 1.5 ml Eppendorf tube comprising 1X CutSmart Buffer, 200 units of the restriction enzyme SpeI (New England Biolabs, Cat #R31331) in 100 μl total volume. The plasmid DNA was linearized by incubating at 37° C. overnight to ensure complete digestion.

The linearized plasmid was purified using a DNA QIAquick PCR purification kit (Qiagen, Cat #28104) according to the manufacturer's instructions, and eluting the purified DNA in 40 μl of nuclease free water. The DNA concentration of the eluate was determined using a NanoDrop microvolume spectrophotometer (ThermoFisher) in accordance with the manufacturer's instructions.

The purified plasmid was used as a DNA template to produce mRNA using the in vitro transcription mMESSAGE mMACHINE T7 Transcription Kit (ThermoFisher, Cat # AM1344) in accordance with the manufacturer's instructions. Briefly, 100 mM stocks of the nucleotides GTP, ATP, CTP and UTP were prepared and 15 μl of each stock (2X final) was transferred to an Eppendorf tube containing 2X (12 μl) of CleanCap® Reagent AG (m7G (5′) ppp (5′) (2′OMeA) pG, TriLink, Cat #N-7113) in a 100 μl total volume.

Approximately 1.67 μg of linear pRT-HA-SPB-CC-AG DNA, 20 μl of 10X T7 Transcription Buffer and 20 μl of T7 RNA polymerase mix was added to a 1.5 ml Eppendorf tube (200 μl final volume), and the tube was incubated at 37° C. for 3 hours. A 10 μl aliquot of TURBO DNase enzyme (ThermoFisher) was added and the tube further incubated at 37° C. for 15 min to degrade the DNA template.

A poly (A) tail was added to the 3′end of the 5′-CleanCap®-HA-SPB mRNA using a Poly (A) Tailing Kit (ThermoFisher, cat #1350M). Briefly, the digested DNA, mRNA solution (210 μl), 100 μl of 25 mM ATP, 200 μl 5X-E-PAP Buffer, 100 μl of 25 mM MnC12, 40 μl of E. coli Poly (A) Polymerase (“E-PAP”), and 350 μl of nuclease-free water (1 ml total volume) were combined and the reaction was allowed to proceed at 37° C. for 1 hr.

The 5′-CleanCap®-HA-SPB-poly (A) mRNA was purified using a RNeasy Midi Purification Kit (Qiagen, Cat #75144) according to the manufacturer's instructions. Briefly, a 3.5 ml solution of Buffer RLT was freshly prepared using 35 μl of 2-mercaptoethanol and combined with 2.5 ml of 100% ethanol, and the final mRNA product was eluted from the column using 300 μl of nuclease-free water. The average mRNA yield from this process is about 600-800 μg.

### Example 12

### Improved PiggyBac Transposase Polynucleotides and 35TCC RE ITR Enhance Fold Expansion and Transposition Efficiency of Gene Edited Cells During Manufacturing of CAR-T Cells

The effect of mRNA encoding piggyBac transposase comprising four hyperactive was tested on the in vitro production of CAR-T cells. The results demonstrated an enhancement in CAR-T cell fold expansion and yield of the percent gene edited T-cells in CAR-T populations.

Briefly, the mRNA encoding SPBv3.0 or SPBv4.0 was nucleofected into pan T-cells isolated from four separate donors. T-cells were co-nucleofected with: a) a transposon comprising: a wild type LE piggyBac ITR and a RE piggyBac ITR comprising a 35TCC mutation: a sequence encoding an anti-MUCIC CAR, an iCAS9 safety switch (further described in International Patent Application Publication No. WO 2018/068022, incorporated herein in its entirety) and a dihydrofolate reductase selectable marker gene; b) an mRNA encoding Cas-CLOVER v3.0 (SEQ ID NO. 46); c) a gRNA pair targeting beta-2-microglobulin (B2M) and a gRNA pair targeting CD3 to knockout expression of B2M and CD3, respectively; and d) an mRNA encoding a booster molecule for expansion of transfected T-cells. (further described in International Patent Application Publication No. WO 2020/051374, incorporated herein by reference in its entirety).

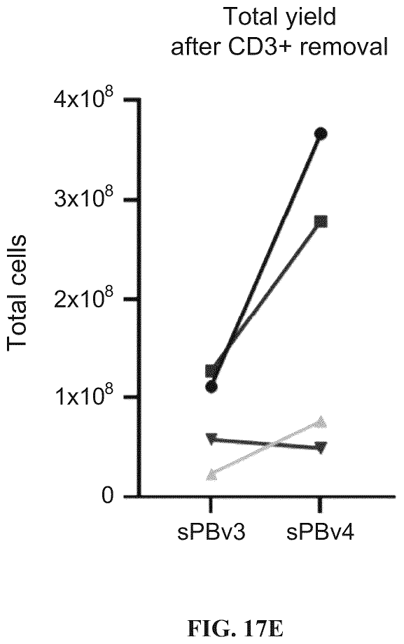

Transfected T-cells from the four donors were expanded using ImmunoCult CD3/CD28/CD2 T Cell Activator, selected for methotrexate resistance, and the yield and fold expansion for activated T-cells from each of the four donors was determined. CD3+ transfected CAR-T-cells were removed by column chromatography using an anti-CD3 beads and T-cells were subject to flow cytometry at Day 5 and 14 to identify T-cell populations: CAR Mean fluorescent intensity (MFI; transposition efficiency [TPE]), percent B2M knockout; percent CD3 knockout and total yield of CD3-T-cells post depletion. The results are shown in FIGS. 17A-17E.

As shown in FIGS. 17A-17E, the mRNA encoding the SPBv4.0 resulted in an increase T-cell expansion in three out of four donors, as much as a 10-fold increase (FIG. 17A), compared to SPBv3.0. While SPBv4.0 did not result in an improvement in overall transpositions efficiency at Day 5 compared to SPBv3.0 (FIG. 17B), SPB4.0 resulted in a greater increase of the percent of B2M knockout cells (FIG. 17C) and CD3-knockout cells (FIG. 17D) and increased the total number of CD3-CAR-T cells in three of the four donors by about 3-4-fold (FIG. 17E). This resulted an increased yield of desired CAR-T cells with knocked out expression of B2M and CD3 in the final CAR-T isolation.

We claim:

1. A polynucleotide, comprising in the 5′ to 3′ direction: (i) a hemoglobin beta (HBB) 5′-UTR, (ii) a sequence encoding a nuclear localization signal (NLS), (iii) a nucleic acid sequence encoding a piggyBac transposase, (iv) a 3′-UTR comprising one or more nucleic acid sequences comprising a human cytochrome b-245 alpha polypeptide (CYBA) 3′-UTR element and one or more miR-142-3p binding sites, and (v) a polyA tail.

2. The polynucleotide of claim 1, wherein the piggyBac transposase comprises the amino acid sequence set forth in SEQ ID NO: 14).

3. The polynucleotide of claim 1 or 2, wherein the nucleic acid sequence encoding the piggyBac transposase comprises the nucleic acid sequence set forth in SEQ ID NO: 2.

4. The polynucleotide of claim 1, wherein the NLS is an SV40 NLS comprising the amino acid sequence set forth in SEQ ID NO: 8.

5. The polynucleotide of claim 1, wherein the one or more CYBA 3′-UTR element(s) each comprises the nucleic acid sequence set forth in SEQ ID NO: 3.

6. The polynucleotide of claim 5, wherein the 3′-UTR comprises at least two tandem nucleic acid sequences encoding a CYBA 3′-UTR element which are separated by a linker sequence.

7. The polynucleotide of claim 6, wherein the tandem nucleic acid sequences encoding a CYBA 3′-UTR element each comprise the nucleic acid sequence set forth in SEQ ID NO. 4.

8. The polynucleotide of claim 1, wherein the one or more miR-142-3p binding sites each comprise the nucleic acid sequence set forth in SEQ ID NO: 5.

9. The polynucleotide of claim 1, wherein each of the one or more miR-142-3p binding sites comprises the nucleic acid sequence ACACTAC.

10. The polynucleotide of claim 9, wherein the 3′-UTR comprises four miR-142-3p binding sites.

11. The polynucleotide of claim 10, further comprising a linker sequence located between each of the four miR-142-3p binding sites.

12. The polynucleotide of claim 11, wherein the linker sequence comprises the nucleic acid sequence set forth in SEQ ID NO. 6.

13. The polynucleotide of claim 1, wherein the HBB 5′-UTR comprises the nucleic acid sequence set forth in SEQ ID NO: 1.

14. The polynucleotide of claim 1, wherein the poly A tail is an 80X polyA tail comprising the nucleic acid sequence set forth in SEQ ID NO: 7.

15. The polynucleotide of claim 1, wherein the polynucleotide comprises the nucleic acid sequence set forth in SEQ ID NO: 10.

16. The polynucleotide of any one of claims 1-15, wherein the polynucleotide is a DNA molecule.

17. The polynucleotide of any one of claims 1-15, wherein the polynucleotide is an RNA molecule.

18. The polynucleotide of claim 17, wherein the RNA molecule is an mRNA molecule.

19. The polynucleotide of claim 18, wherein the mRNA comprises a 5′-CAP.

20. The polynucleotide of claim 19, wherein the 5′CAP is a 5′ CleanCap.

21. A lipid nanoparticle (LNP) composition comprising the polynucleotide of claim 16.

22. A lipid nanoparticle (LNP) composition comprising the polynucleotide of claim 17.

23. A method for the delivery of an exogenous nucleic acid to a cell, comprising:
introducing into the cell a DNA transposon comprising an exogenous nucleic acid; and an mRNA comprising in 5′ to 3′ direction: (i) a HBB 5′-UTR, a nucleic acid sequence encoding an NLS, (ii) a nucleic acid sequence encoding a piggyBac transposase comprising five hyperactive mutations, (iii) a 3′-UTR comprising two or more tandem nucleic acid sequences comprising a CYBA 3′-UTR element and four miR-142-3p binding sites, and (iv) a polyA tail;
wherein the piggyBac transposase is expressed in the cell and integrates the transposon comprising the exogenous nucleic acid at a TTAA integration site in the cell genome, wherein the expressed piggyBac transposase exhibits enhanced integration and/or excision activity compared to a piggyBac transposase comprising four or fewer hyperactive mutations.

24. A method for the in vivo delivery of an exogenous nucleic acid to a cell in a subject, comprising: co-introducing into the subject a DNA transposon comprising an exogenous nucleic acid; and an mRNA comprising in the 5′ to 3′ direction: a HBB 5′-UTR, a NLS coding sequence, a piggyBac transposase coding sequence comprising five hyperactive mutations, a 3′-UTR comprising tandem CYBA 3′-UTR element upstream of the sequences for four miR-142-3p binding sites, and a polyA tail;
wherein a cell in the subject uptakes the transposon and expresses the piggyBac transposase in the cell and integrates the transposon comprising the exogenous nucleic acid at a TTAA integration site in the cell genome of the subject, wherein the expressed piggyBac transposase exhibits reduced immunogenicity in the subject compared to a mRNA encoding a piggyBac transposase lacking the sequences for four miR-142-3p binding sites.


SEQUENCE LISTING (61 sequences)

SEQ ID NO: 1 - DNA, 50 bp, synthetic construct
  source 1..50: mol_type = other DNA
acatttgcttctgacacaactgtgttcactagcaacctcaaacagacacc

SEQ ID NO: 2 - DNA, 1818 bp, synthetic construct
  source 1..1818: mol_type = other DNA
atggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggttcgacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttctga

SEQ ID NO: 3 - DNA, 64 bp, synthetic construct
  source 1..64: mol_type = other DNA
cctcgccccggacctgccctcccgccaggtgcacccacctgcaataaatgcagcgaagccggga

SEQ ID NO: 4 - DNA, 134 bp, synthetic construct
  source 1..134: mol_type = other DNA
cctcgccccggacctgccctcccgccaggtgcacccacctgcaataaatgcagcgaagccgggagaattccctcgccccggacctgccctcccgccaggtgcacccacctgcaataaatgcagcgaagccggga

SEQ ID NO: 5 - DNA, 23 bp, synthetic construct
  source 1..23: mol_type = other DNA
tccataaagtaggaaacactaca

SEQ ID NO: 6 - DNA, 106 bp, synthetic construct
  source 1..106: mol_type = other DNA
tccataaagtaggaaacactacacgattccataaagtaggaaacactacaaccggttccataaagtaggaaacactacatcactccataaagtaggaaacactaca

SEQ ID NO: 7 - DNA, 80 bp, synthetic construct
  source 1..80: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - protein, 7 aa, synthetic construct
PKKKRKV

SEQ ID NO: 9 - DNA, 21 bp, synthetic construct
  source 1..21: mol_type = other DNA
cccaagaagaagcggaaagtt

SEQ ID NO: 10 - DNA, 2248 bp, synthetic construct
  source 1..2248: mol_type = other DNA
acatttgcttctgacacaactgtgttcactagcaacctcaaacagacaccaagcttgccaccatggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggttcgacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttctgagcggccgccctcgccccggacctgccctcccgccaggtgcacccacctgcaataaatgcagcgaagccgggagaattccctcgccccggacctgccctcccgccaggtgcacccacctgcaataaatgcagcgaagccgggacgaattcctccataaagtaggaaacactacacgattccataaagtaggaaacactacaaccggttccataaagtaggaaacactacatcactccataaagtaggaaacactacacgatatctctatagtgtcacctaaatttaattaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - DNA, 35 bp, synthetic construct
  source 1..35: mol_type = other DNA
ccctagaaagatagtctgcgtaaaattgacgcatg

SEQ ID NO: 12 - DNA, 63 bp, synthetic construct
  source 1..63: mol_type = other DNA
ccctagaaagataatcatattgtgacgtacgttaaagataatcatgcgtaaaattgacgcatg

SEQ ID NO: 13 - DNA, 309 bp, synthetic construct
  source 1..309: mol_type = other DNA
gatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgtcatttagaaagagagagcaatatttcaagaatgcaggagtcaattttacgcagactatctttctaggg

SEQ ID NO: 14 - DNA, 35 bp, synthetic construct; identical to SEQ ID NO: 11
  source 1..35: mol_type = other DNA

SEQ ID NO: 15 - DNA, 63 bp, synthetic construct; identical to SEQ ID NO: 12
  source 1..63: mol_type = other DNA

SEQ ID NO: 16 - DNA, 44 bp, synthetic construct
  source 1..44: mol_type = other DNA
ccctagaaagataatcatattgtgacgtacgttaaagataatca

SEQ ID NO: 17 - DNA, 39 bp, synthetic construct
  source 1..39: mol_type = other DNA
ccctagaaagataatcatattgtgacgtacgttaaagat

SEQ ID NO: 18 - DNA, 34 bp, synthetic construct
  source 1..34: mol_type = other DNA
ccctagaaagataatcatattgtgacgtacgtta

SEQ ID NO: 19 - DNA, 29 bp, synthetic construct
  source 1..29: mol_type = other DNA
ccctagaaagataatcatattgtgacgta

SEQ ID NO: 20 - DNA, 24 bp, synthetic construct
  source 1..24: mol_type = other DNA
ccctagaaagataatcatattgtg

SEQ ID NO: 21 - DNA, 19 bp, synthetic construct
  source 1..19: mol_type = other DNA
ccctagaaagataatcata

SEQ ID NO: 22 - DNA, 17 bp, synthetic construct
  source 1..17: mol_type = other DNA
ccctagaaagataatca

SEQ ID NO: 23 - DNA, 16 bp, synthetic construct
  source 1..16: mol_type = other DNA
ccctagaaagataatc

SEQ ID NO: 24 - DNA, 18 bp, synthetic construct
  source 1..18: mol_type = other DNA
ccctagaaagataatcat

SEQ ID NO: 25 - DNA, 20 bp, synthetic construct
  source 1..20: mol_type = other DNA
ccctagaaagataatcatat

SEQ ID NO: 26 - DNA, 21 bp, synthetic construct
  source 1..21: mol_type = other DNA
ccctagaaagataatcatatt

SEQ ID NO: 27 - DNA, 22 bp, synthetic construct
  source 1..22: mol_type = other DNA
ccctagaaagataatcatattg

SEQ ID NO: 28 - DNA, 23 bp, synthetic construct
  source 1..23: mol_type = other DNA
ccctagaaagataatcatattgt

SEQ ID NO: 29 - DNA, 25 bp, synthetic construct
  source 1..25: mol_type = other DNA
ccctagaaagataatcatattgtga

SEQ ID NO: 30 - DNA, 26 bp, synthetic construct
  source 1..26: mol_type = other DNA
ccctagaaagataatcatattgtgac

SEQ ID NO: 31 - DNA, 27 bp, synthetic construct
  source 1..27: mol_type = other DNA
ccctagaaagataatcatattgtgacg

SEQ ID NO: 32 - DNA, 28 bp, synthetic construct
  source 1..28: mol_type = other DNA
ccctagaaagataatcatattgtgacgt

SEQ ID NO: 33 - DNA, 30 bp, synthetic construct
  source 1..30: mol_type = other DNA
ccctagaaagataatcatattgtgacgtac

SEQ ID NO: 34 - DNA, 31 bp, synthetic construct
  source 1..31: mol_type = other DNA
ccctagaaagataatcatattgtgacgtacg

SEQ ID NO: 35 - DNA, 32 bp, synthetic construct
  source 1..32: mol_type = other DNA
ccctagaaagataatcatattgtgacgtacgt

SEQ ID NO: 36 - DNA, 33 bp, synthetic construct
  source 1..33: mol_type = other DNA
ccctagaaagataatcatattgtgacgtacgtt

SEQ ID NO: 37 - DNA, 35 bp, synthetic construct
  source 1..35: mol_type = other DNA
ccctagaaagataatcatattgtgacgtacgttaa

SEQ ID NO: 38 - DNA, 36 bp, synthetic construct
  source 1..36: mol_type = other DNA
ccctagaaagataatcatattgtgacgtacgttaaa

SEQ ID NO: 39 - DNA, 37 bp, synthetic construct
  source 1..37: mol_type = other DNA
ccctagaaagataatcatattgtgacgtacgttaaag

SEQ ID NO: 40 - DNA, 38 bp, synthetic construct
  source 1..38: mol_type = other DNA
ccctagaaagataatcatattgtgacgtacgttaaaga

SEQ ID NO: 41 - DNA, 40 bp, synthetic construct
  source 1..40: mol_type = other DNA
ccctagaaagataatcatattgtgacgtacgttaaagata

SEQ ID NO: 42 - DNA, 41 bp, synthetic construct
  source 1..41: mol_type = other DNA
ccctagaaagataatcatattgtgacgtacgttaaagataa

SEQ ID NO: 43 - DNA, 42 bp, synthetic construct
  source 1..42: mol_type = other DNA
ccctagaaagataatcatattgtgacgtacgttaaagataat

SEQ ID NO: 44 - DNA, 43 bp, synthetic construct
  source 1..43: mol_type = other DNA
ccctagaaagataatcatattgtgacgtacgttaaagataatc

SEQ ID NO: 45 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRFDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 46 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 47 - skipped

SEQ ID NO: 48 - protein, 1053 aa, synthetic construct
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 49 - protein, 1368 aa, synthetic construct
XDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 50 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 51 - protein, 199 aa, synthetic construct
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 52 - protein, 1591 aa, synthetic construct
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 53 - DNA, 4776 bp, synthetic construct
  source 1..4776: mol_type = other DNA
atggcaccaaagaagaaaagaaaagtggagggcatcaagtcaaacatcagcctgctgaaagacgaactgcggggacagattagtcacatcagtcacgagtacctgtcactgattgatctggccttcgacagcaagcagaatagactgtttgagatgaaagtgctggaactgctggtcaacgagtatggcttcaagggcagacatctgggcgggtctaggaaacctgacggcatcgtgtacagtaccacactggaagacaacttcggaatcattgtcgataccaaggcttattccgagggctactctctgccaattagtcaggcagatgagatggaaaggtacgtgcgcgaaaactcaaatagggacgaggaagtcaaccccaataagtggtgggagaatttcagcgaggaagtgaagaaatactacttcgtctttatctcaggcagcttcaaagggaagtttgaggaacagctgcggagactgtccatgactaccggggtgaacggatctgctgtcaacgtggtcaatctgctgctgggcgcagaaaagatcaggtccggggagatgacaattgaggaactggaacgcgccatgttcaacaattctgagtttatcctgaagtatggaggcgggggaagcgataagaaatactccatcggactggccattggcaccaattccgtgggctgggctgtcatcacagacgagtacaaggtgccaagcaagaagttcaaggtcctggggaacaccgatcgccacagtatcaagaaaaatctgattggagccctgctgttcgactcaggcgagactgctgaagcaacccgactgaagcggactgctaggcgccgatatacccggagaaaaaatcggatctgctacctgcaggaaattttcagcaacgagatggccaaggtggacgatagtttctttcaccgcctggaggaatcattcctggtggaggaagataagaaacacgagcggcatcccatctttggcaacattgtggacgaagtcgcttatcacgagaagtaccctactatctatcatctgaggaagaaactggtggactccaccgataaggcagacctgcgcctgatctatctggccctggctcacatgatcaagttccgggggcattttctgatcgagggagatctgaaccctgacaattctgatgtggacaagctgttcatccagctggtccagacatacaatcagctgtttgaggaaaacccaattaatgcctcaggcgtggacgcaaaggccatcctgagcgccagactgtccaaatctaggcgcctggaaaacctgatcgctcagctgccaggagagaagaaaaacggcctgtttgggaatctgattgcactgtccctgggcctgacacccaacttcaagtctaattttgatctggccgaggacgctaagctgcagctgtccaaagacacttatgacgatgacctggataacctgctggctcagatcggcgatcagtacgcagacctgttcctggccgctaagaatctgagtgacgccatcctgctgtcagatattctgcgcgtgaacacagagattactaaggccccactgagtgcttcaatgatcaaaagatatgacgagcaccatcaggatctgaccctgctgaaggctctggtgaggcagcagctgcccgagaaatacaaggaaatcttctttgatcagagcaagaatggatacgccggctatattgacggcggggcttcccaggaggagttctacaagttcatcaagcccattctggaaaagatggacggcaccgaggaactgctggtgaagctgaatcgggaggacctgctgagaaaacagaggacatttgataacggaagcatccctcaccagattcatctgggcgaactgcacgccatcctgcgacggcaggaggacttctacccatttctgaaggataaccgcgagaaaatcgaaaagatcctgaccttcagaatcccctactatgtggggcctctggcacggggaaatagtagatttgcctggatgacaagaaagtcagaggaaactatcaccccctggaacttcgaggaagtggtcgataaaggcgctagcgcacagtccttcattgaaaggatgacaaattttgacaagaacctgccaaatgagaaggtgctgcccaaacacagcctgctgtacgaatatttcacagtgtataacgagctgactaaagtgaagtacgtcaccgaagggatgcgcaagcccgcattcctgtccggagagcagaagaaagccatcgtggacctgctgtttaagacaaatcggaaagtgactgtcaaacagctgaaggaagactatttcaagaaaattgagtgtttcgattcagtggaaatcagcggcgtcgaggacaggtttaacgcctccctggggacctaccacgatctgctgaagatcatcaaggataaggacttcctggacaacgaggaaaatgaggacatcctggaggacattgtgctgacactgactctgtttgaggatcgcgaaatgatcgaggaacgactgaagacttatgcccatctgttcgatgacaaagtgatgaagcagctgaaaagaaggcgctacaccggatggggacgcctgagccgaaaactgatcaatgggattagagacaagcagagcggaaaaactatcctggactttctgaagtccgatggcttcgccaacaggaacttcatgcagctgattcacgatgactctctgaccttcaaggaggacatccagaaagcacaggtgtctggccagggggacagtctgcacgagcatatcgcaaacctggccggcagccccgccatcaagaaagggattctgcagaccgtgaaggtggtggacgaactggtcaaggtcatgggacgacacaaacctgagaacatcgtgattgagatggcccgcgaaaatcagacaactcagaagggccagaaaaacagtcgagaacggatgaagagaatcgaggaaggcatcaaggagctggggtcacagatcctgaaggagcatcctgtggaaaacactcagctgcagaatgagaaactgtatctgtactatctgcagaatggacgggatatgtacgtggaccaggagctggatattaacagactgagtgattatgacgtggatgccatcgtccctcagagcttcctgaaggatgactccattgacaacaaggtgctgaccaggtccgacaagaaccgcggcaaatcagataatgtgccaagcgaggaagtggtcaagaaaatgaagaactactggaggcagctgctgaatgccaagctgatcacacagcggaaatttgataacctgactaaggcagaaagaggaggcctgtctgagctggacaaggccggcttcatcaagcggcagctggtggagacaagacagatcactaagcacgtcgctcagattctggatagcagaatgaacacaaagtacgatgaaaacgacaagctgatcagggaggtgaaagtcattactctgaaatccaagctggtgtctgactttagaaaggatttccagttttataaagtcagggagatcaacaactaccaccatgctcatgacgcatacctgaacgcagtggtcgggaccgccctgattaagaaataccccaagctggagtccgagttcgtgtacggagactataaagtgtacgatgtccggaagatgatcgccaaatctgagcaggaaattggcaaggccaccgctaagtatttcttttacagtaacatcatgaatttctttaagaccgaaatcacactggcaaatggggagatcagaaaaaggcctctgattgagaccaacggggagacaggagaaatcgtgtgggacaagggaagggattttgctaccgtgcgcaaagtcctgtccatgccccaagtgaatattgtcaagaaaactgaagtgcagaccgggggattctctaaggagagtattctgcctaagcgaaactctgataaactgatcgcccggaagaaagactgggaccccaagaagtatggcgggttcgactctccaacagtggcttacagtgtcctggtggtcgcaaaggtggaaaaggggaagtccaagaaactgaagtctgtcaaagagctgctgggaatcactattatggaacgcagctccttcgagaagaatcctatcgattttctggaagccaagggctataaagaggtgaagaaagacctgatcattaagctgccaaaatactcactgtttgagctggaaaacggacgaaagcgaatgctggcaagcgccggagaactgcagaagggcaatgagctggccctgccctccaaatacgtgaacttcctgtatctggctagccactacgagaaactgaaggggtcccctgaggataacgaacagaagcagctgtttgtggagcagcacaaacattatctggacgagatcattgaacagatttcagagttcagcaagagagtgatcctggctgacgcaaatctggataaagtcctgagcgcatacaacaagcaccgagacaaaccaatccgggagcaggccgaaaatatcattcatctgttcaccctgacaaacctgggcgcccctgcagccttcaagtattttgacaccacaatcgatcggaagagatacacttctaccaaagaggtgctggatgctaccctgatccaccagagtattaccggcctgtatgagacacgcatcgacctgtcacagctgggaggcgatgggagccccaagaaaaagcggaaggtgtctagttaa

SEQ ID NO: 54 - protein, 1588 aa, synthetic construct
MPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 55 - DNA, 4767 bp, synthetic construct
  source 1..4767: mol_type = other DNA
atgcctaagaagaagcggaaggtggaaggcatcaaaagcaacatctccctcctgaaagacgaactccgggggcagattagccacattagtcacgaatacctctccctcatcgacctggctttcgatagcaagcagaacaggctctttgagatgaaagtgctggaactgctcgtcaatgagtacgggttcaagggtcgacacctcggcggatctaggaaaccagacggcatcgtgtatagtaccacactggaagacaactttgggatcattgtggataccaaggcatactctgagggttatagtctgcccatttcacaggccgacgagatggaacggtacgtgcgcgagaactcaaatagagatgaggaagtcaaccctaacaagtggtgggagaacttctctgaggaagtgaagaaatactacttcgtctttatcagcgggtccttcaagggtaaatttgaggaacagctcaggagactgagcatgactaccggcgtgaatggcagcgccgtcaacgtggtcaatctgctcctgggcgctgaaaagattcggagcggagagatgaccatcgaagagctggagagggcaatgtttaataatagcgagtttatcctgaaatacggtggcggtggatccgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctga

SEQ ID NO: 56 - protein, 1591 aa, synthetic construct; identical to SEQ ID NO: 52

SEQ ID NO: 57 - RNA, 4779 nt, synthetic construct
  source 1..4779: mol_type = other RNA
atggctcccaagaagaagcggaaggtcgagggcatcaagagcaacatcagcctgctgaaggacgagctgagaggccagatcagccacatctcccacgagtacctgagcctgatcgacctggccttcgaccccaagcagaaccggctgttcgagatgaaggtgctggaactgctggtcaacgagtacggcttcaagggcagacacctcggcggcagcagaaagcctgatggcatcgtgtacagcaccacactcgaggacaacttcggcatcatcgtggacaccaaggcctacagcgagggctacagcctgcctatctctcaggccgacgagatggaaagatacgtgcgcgagaacagcaaccgcgacgaggaagtgaaccccaacaagtggtgggagaacttcagcgaggaagtcaaaaagtactacttcgtgttcatcagcggcagctttaagggcaagttcgaggaacagctgcggcggctgtctatgaccacaggcgttaacggcagcgccgtgaacgtggtcaatctgctgctgggcgccgagaagattagaagcggcgagatgaccatcgaggaactggaacgggccatgttcaacaacagcgagttcatcctgaagtacggcggaggcggcagcgacaagaagtactctatcggactggccatcggcaccaactctgttggatgggccgtgatcaccgacgagtacaaggtgcccagcaagaaattcaaagtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggacaagaagcacgagcggcaccccatcttcggaaatatcgtggacgaggtggcctaccacgagaagtaccccaccatctaccacctgagaaagaaactggtggacagcaccgacaaggccgacctgcgactgatctatctggccctggctcacatgatcaagttccggggccacttcctgatcgagggcgacctgaatcctgacaactccgacgtggacaagctgttcatccagctggtgcagacctacaatcagctgttcgaagagaatcccatcaacgcctctggcgtggacgccaaagccatcctgtctgccagactgagcaagagcagacggctggaaaacctgatcgctcagctgcccggcgagaagaagaatggcctgttcggcaacctgattgccctgtctctgggcctgacacctaacttcaagtccaacttcgatctggccgaggatgccaaactgcagctgtccaaggacacctacgacgacgacctggataacctgctggcccagatcggcgatcagtacgccgacttgtttctggccgccaagaacctgtctgacgccatcctgctgagcgacatcctgagagtgaacaccgagatcacaaaggcccctctgagcgcctctatgatcaagagatacgacgagcaccaccaggatctgaccctgctgaaagctctcgtcaggcagcagctgccagagaagtacaaagagattttcttcgaccagagcaagaacggctacgccggctacattgatggcggagccagccaagaggaattctacaagttcatcaagcccatcctcgagaagatggacggcacagaggaactgctcgtgaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaagaggtggtggacaagggcgccagcgctcagtccttcatcgagcggatgaccaatttcgacaagaatctgcccaacgagaaagtgctgcccaagcactccctgctgtacgagtacttcaccgtgtacaacgagctgaccaaagtgaaatacgtgaccgagggaatgagaaagcccgcctttctgtccggcgagcagaaaaaggccatcgtggatctgctgttcaagaccaaccggaaagtgaccgtgaagcagctgaaagaggactacttcaagaaaatcgagtgcttcgactccgtggaaatcagcggcgtggaagatcggttcaatgccagcctgggcacataccacgatctgctgaaaattatcaaggacaaggacttcctggacaacgaggaaaacgaggacatccttgaggacatcgtgctgaccctgacactgttcgaggacagagagatgatcgaggaaaggctgaaaacatacgcccacctgttcgacgacaaagtcatgaagcaactgaagcggcggcgctacacaggctggggcagactgtctagaaagctgatcaacggcatccgggacaagcagtccggcaagaccatcctggactttctgaagtccgacggcttcgccaacagaaacttcatgcagctgattcacgacgacagcctcaccttcaaagaggacattcagaaggcccaggtttccggccagggcgattctctgcacgagcacattgccaatctggccggctctcccgccattaagaagggcattctgcagacagtgaaagtggtggatgagctggtcaaagtgatggggagacacaagcccgagaacatcgtgatcgaaatggccagagagaaccagaccacacagaagggccagaagaactcccgcgagagaatgaagcggatcgaagagggaatcaaagagctggggagccagatcctgaaagaacaccccgtggaaaacacccagctgcagaacgagaagctgtacctgtactacctccagaacggccgggatatgtacgtggaccaagagctggacatcaaccgcctgagcgactacgatgtggacgctatcgtgccccagtcttttctgaaagatgactccatcgacaacaaggtgctgaccagaagcgataagaaccggggcaagagcgacaacgtgccctctgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacaccaaatacgatgagaacgacaaactgatccgggaagtgaaagtcatcaccctgaagtccaagctggtgtccgatttccggaaggatttccagttctacaaagtgcgggaaatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggaaaggctaccgccaaatacttcttctactccaacatcatgaactttttcaagacagagatcaccctcgccaacggcgagatcagaaagcggcctctgatcgagacaaacggcgaaaccggcgagattgtgtgggataagggcagagactttgccacagtgcggaaggtgctcagcatgccccaagtgaatatcgtgaaaaagaccgaggtgcagacaggcggcttcagcaaagagtccattctgcctaagcggaactccgacaagctgatcgcccggaagaaggactgggaccccaagaaatacggcggcttcgatagccctaccgtggcctattctgtgctggtggtggccaaagtggaaaagggaaagtccaagaagctcaagagcgtcaaagaactcctgggcatcaccatcatggaacggtccagcttcgagaagaaccctatcgactttctggaagccaagggctacaaagaagtcaagaaggacctgatcatcaagctccccaagtacagcctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcagaagcagcttttcgtcgagcagcacaagcactacctggacgagatcatcgagcagatctccgagttctccaagagagtgatcctggccgacgccaacctggacaaggttctgtccgcctacaacaagcaccgggataagcccatcagagagcaggccgagaatatcatccacctgtttaccctgaccaacctgggagcccctgccgccttcaagtacttcgacaccaccatcgaccggaagcgctacaccagcaccaaagaagtgctggacgccacactgatccaccagagcatcaccggcctgtacgagacacggatcgatctgtctcagcttggaggcgacggcagccctaagaagaagagaaaggtttccagctaataa

SEQ ID NO: 58 - protein, 1591 aa, synthetic construct
MAPKKKRKVEGIKSNISLLKDELRGQISHISHEYLSLIDLAFDPKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 59 - RNA, 5124 nt, synthetic construct
  source 1..5124: mol_type = other RNA
acatttgcttctgacacaactgtgttcactagcaacctcaaacagacaccaagcttgccaccatggctcccaagaagaagcggaaggtcgagggcatcaagagcaacatcagcctgctgaaggacgagctgagaggccagatcagccacatctcccacgagtacctgagcctgatcgacctggccttcgaccccaagcagaaccggctgttcgagatgaaggtgctggaactgctggtcaacgagtacggcttcaagggcagacacctcggcggcagcagaaagcctgatggcatcgtgtacagcaccacactcgaggacaacttcggcatcatcgtggacaccaaggcctacagcgagggctacagcctgcctatctctcaggccgacgagatggaaagatacgtgcgcgagaacagcaaccgcgacgaggaagtgaaccccaacaagtggtgggagaacttcagcgaggaagtcaaaaagtactacttcgtgttcatcagcggcagctttaagggcaagttcgaggaacagctgcggcggctgtctatgaccacaggcgttaacggcagcgccgtgaacgtggtcaatctgctgctgggcgccgagaagattagaagcggcgagatgaccatcgaggaactggaacgggccatgttcaacaacagcgagttcatcctgaagtacggcggaggcggcagcgacaagaagtactctatcggactggccatcggcaccaactctgttggatgggccgtgatcaccgacgagtacaaggtgcccagcaagaaattcaaagtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggacaagaagcacgagcggcaccccatcttcggaaatatcgtggacgaggtggcctaccacgagaagtaccccaccatctaccacctgagaaagaaactggtggacagcaccgacaaggccgacctgcgactgatctatctggccctggctcacatgatcaagttccggggccacttcctgatcgagggcgacctgaatcctgacaactccgacgtggacaagctgttcatccagctggtgcagacctacaatcagctgttcgaagagaatcccatcaacgcctctggcgtggacgccaaagccatcctgtctgccagactgagcaagagcagacggctggaaaacctgatcgctcagctgcccggcgagaagaagaatggcctgttcggcaacctgattgccctgtctctgggcctgacacctaacttcaagtccaacttcgatctggccgaggatgccaaactgcagctgtccaaggacacctacgacgacgacctggataacctgctggcccagatcggcgatcagtacgccgacttgtttctggccgccaagaacctgtctgacgccatcctgctgagcgacatcctgagagtgaacaccgagatcacaaaggcccctctgagcgcctctatgatcaagagatacgacgagcaccaccaggatctgaccctgctgaaagctctcgtcaggcagcagctgccagagaagtacaaagagattttcttcgaccagagcaagaacggctacgccggctacattgatggcggagccagccaagaggaattctacaagttcatcaagcccatcctcgagaagatggacggcacagaggaactgctcgtgaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaagaggtggtggacaagggcgccagcgctcagtccttcatcgagcggatgaccaatttcgacaagaatctgcccaacgagaaagtgctgcccaagcactccctgctgtacgagtacttcaccgtgtacaacgagctgaccaaagtgaaatacgtgaccgagggaatgagaaagcccgcctttctgtccggcgagcagaaaaaggccatcgtggatctgctgttcaagaccaaccggaaagtgaccgtgaagcagctgaaagaggactacttcaagaaaatcgagtgcttcgactccgtggaaatcagcggcgtggaagatcggttcaatgccagcctgggcacataccacgatctgctgaaaattatcaaggacaaggacttcctggacaacgaggaaaacgaggacatccttgaggacatcgtgctgaccctgacactgttcgaggacagagagatgatcgaggaaaggctgaaaacatacgcccacctgttcgacgacaaagtcatgaagcaactgaagcggcggcgctacacaggctggggcagactgtctagaaagctgatcaacggcatccgggacaagcagtccggcaagaccatcctggactttctgaagtccgacggcttcgccaacagaaacttcatgcagctgattcacgacgacagcctcaccttcaaagaggacattcagaaggcccaggtttccggccagggcgattctctgcacgagcacattgccaatctggccggctctcccgccattaagaagggcattctgcagacagtgaaagtggtggatgagctggtcaaagtgatggggagacacaagcccgagaacatcgtgatcgaaatggccagagagaaccagaccacacagaagggccagaagaactcccgcgagagaatgaagcggatcgaagagggaatcaaagagctggggagccagatcctgaaagaacaccccgtggaaaacacccagctgcagaacgagaagctgtacctgtactacctccagaacggccgggatatgtacgtggaccaagagctggacatcaaccgcctgagcgactacgatgtggacgctatcgtgccccagtcttttctgaaagatgactccatcgacaacaaggtgctgaccagaagcgataagaaccggggcaagagcgacaacgtgccctctgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacaccaaatacgatgagaacgacaaactgatccgggaagtgaaagtcatcaccctgaagtccaagctggtgtccgatttccggaaggatttccagttctacaaagtgcgggaaatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggaaaggctaccgccaaatacttcttctactccaacatcatgaactttttcaagacagagatcaccctcgccaacggcgagatcagaaagcggcctctgatcgagacaaacggcgaaaccggcgagattgtgtgggataagggcagagactttgccacagtgcggaaggtgctcagcatgccccaagtgaatatcgtgaaaaagaccgaggtgcagacaggcggcttcagcaaagagtccattctgcctaagcggaactccgacaagctgatcgcccggaagaaggactgggaccccaagaaatacggcggcttcgatagccctaccgtggcctattctgtgctggtggtggccaaagtggaaaagggaaagtccaagaagctcaagagcgtcaaagaactcctgggcatcaccatcatggaacggtccagcttcgagaagaaccctatcgactttctggaagccaagggctacaaagaagtcaagaaggacctgatcatcaagctccccaagtacagcctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcagaagcagcttttcgtcgagcagcacaagcactacctggacgagatcatcgagcagatctccgagttctccaagagagtgatcctggccgacgccaacctggacaaggttctgtccgcctacaacaagcaccgggataagcccatcagagagcaggccgagaatatcatccacctgtttaccctgaccaacctgggagcccctgccgccttcaagtacttcgacaccaccatcgaccggaagcgctacaccagcaccaaagaagtgctggacgccacactgatccaccagagcatcaccggcctgtacgagacacggatcgatctgtctcagcttggaggcgacggcagccctaagaagaagagaaaggtttccagctaataaggcggccgccctcgccccggacctgccctcccgccaggtgcacccacctgcaataaatgcagcgaagccgggagaattccctcgccccggacctgccctcccgccaggtgcacccacctgcaataaatgcagcgaagccgggagcggccgcggatccccgggtaccgaattcgatatctctatagtgtcacctaaatttaattaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 60 - protein, 5 aa, synthetic construct
GGGGS

SEQ ID NO: 61 - DNA, 4779 bp, synthetic construct
  source 1..4779: mol_type = other DNA
atggctcccaagaagaagcggaaggtcgagggcatcaagagcaacatcagcctgctgaaggacgagctgagaggccagatcagccacatctcccacgagtacctgagcctgatcgacctggccttcgaccccaagcagaaccggctgttcgagatgaaggtgctggaactgctggtcaacgagtacggcttcaagggcagacacctcggcggcagcagaaagcctgatggcatcgtgtacagcaccacactcgaggacaacttcggcatcatcgtggacaccaaggcctacagcgagggctacagcctgcctatctctcaggccgacgagatggaaagatacgtgcgcgagaacagcaaccgcgacgaggaagtgaaccccaacaagtggtgggagaacttcagcgaggaagtcaaaaagtactacttcgtgttcatcagcggcagctttaagggcaagttcgaggaacagctgcggcggctgtctatgaccacaggcgttaacggcagcgccgtgaacgtggtcaatctgctgctgggcgccgagaagattagaagcggcgagatgaccatcgaggaactggaacgggccatgttcaacaacagcgagttcatcctgaagtacggcggaggcggcagcgacaagaagtactctatcggactggccatcggcaccaactctgttggatgggccgtgatcaccgacgagtacaaggtgcccagcaagaaattcaaagtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggacaagaagcacgagcggcaccccatcttcggaaatatcgtggacgaggtggcctaccacgagaagtaccccaccatctaccacctgagaaagaaactggtggacagcaccgacaaggccgacctgcgactgatctatctggccctggctcacatgatcaagttccggggccacttcctgatcgagggcgacctgaatcctgacaactccgacgtggacaagctgttcatccagctggtgcagacctacaatcagctgttcgaagagaatcccatcaacgcctctggcgtggacgccaaagccatcctgtctgccagactgagcaagagcagacggctggaaaacctgatcgctcagctgcccggcgagaagaagaatggcctgttcggcaacctgattgccctgtctctgggcctgacacctaacttcaagtccaacttcgatctggccgaggatgccaaactgcagctgtccaaggacacctacgacgacgacctggataacctgctggcccagatcggcgatcagtacgccgacttgtttctggccgccaagaacctgtctgacgccatcctgctgagcgacatcctgagagtgaacaccgagatcacaaaggcccctctgagcgcctctatgatcaagagatacgacgagcaccaccaggatctgaccctgctgaaagctctcgtcaggcagcagctgccagagaagtacaaagagattttcttcgaccagagcaagaacggctacgccggctacattgatggcggagccagccaagaggaattctacaagttcatcaagcccatcctcgagaagatggacggcacagaggaactgctcgtgaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaagaggtggtggacaagggcgccagcgctcagtccttcatcgagcggatgaccaatttcgacaagaatctgcccaacgagaaagtgctgcccaagcactccctgctgtacgagtacttcaccgtgtacaacgagctgaccaaagtgaaatacgtgaccgagggaatgagaaagcccgcctttctgtccggcgagcagaaaaaggccatcgtggatctgctgttcaagaccaaccggaaagtgaccgtgaagcagctgaaagaggactacttcaagaaaatcgagtgcttcgactccgtggaaatcagcggcgtggaagatcggttcaatgccagcctgggcacataccacgatctgctgaaaattatcaaggacaaggacttcctggacaacgaggaaaacgaggacatccttgaggacatcgtgctgaccctgacactgttcgaggacagagagatgatcgaggaaaggctgaaaacatacgcccacctgttcgacgacaaagtcatgaagcaactgaagcggcggcgctacacaggctggggcagactgtctagaaagctgatcaacggcatccgggacaagcagtccggcaagaccatcctggactttctgaagtccgacggcttcgccaacagaaacttcatgcagctgattcacgacgacagcctcaccttcaaagaggacattcagaaggcccaggtttccggccagggcgattctctgcacgagcacattgccaatctggccggctctcccgccattaagaagggcattctgcagacagtgaaagtggtggatgagctggtcaaagtgatggggagacacaagcccgagaacatcgtgatcgaaatggccagagagaaccagaccacacagaagggccagaagaactcccgcgagagaatgaagcggatcgaagagggaatcaaagagctggggagccagatcctgaaagaacaccccgtggaaaacacccagctgcagaacgagaagctgtacctgtactacctccagaacggccgggatatgtacgtggaccaagagctggacatcaaccgcctgagcgactacgatgtggacgctatcgtgccccagtcttttctgaaagatgactccatcgacaacaaggtgctgaccagaagcgataagaaccggggcaagagcgacaacgtgccctctgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacaccaaatacgatgagaacgacaaactgatccgggaagtgaaagtcatcaccctgaagtccaagctggtgtccgatttccggaaggatttccagttctacaaagtgcgggaaatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggaaaggctaccgccaaatacttcttctactccaacatcatgaactttttcaagacagagatcaccctcgccaacggcgagatcagaaagcggcctctgatcgagacaaacggcgaaaccggcgagattgtgtgggataagggcagagactttgccacagtgcggaaggtgctcagcatgccccaagtgaatatcgtgaaaaagaccgaggtgcagacaggcggcttcagcaaagagtccattctgcctaagcggaactccgacaagctgatcgcccggaagaaggactgggaccccaagaaatacggcggcttcgatagccctaccgtggcctattctgtgctggtggtggccaaagtggaaaagggaaagtccaagaagctcaagagcgtcaaagaactcctgggcatcaccatcatggaacggtccagcttcgagaagaaccctatcgactttctggaagccaagggctacaaagaagtcaagaaggacctgatcatcaagctccccaagtacagcctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcagaagcagcttttcgtcgagcagcacaagcactacctggacgagatcatcgagcagatctccgagttctccaagagagtgatcctggccgacgccaacctggacaaggttctgtccgcctacaacaagcaccgggataagcccatcagagagcaggccgagaatatcatccacctgtttaccctgaccaacctgggagcccctgccgccttcaagtacttcgacaccaccatcgaccggaagcgctacaccagcaccaaagaagtgctggacgccacactgatccaccagagcatcaccggcctgtacgagacacggatcgatctgtctcagcttggaggcgacggcagccctaagaagaagagaaaggtttccagctaataa